(19) United States
(12) Patent Application Publication
(10) Pub. No.: US 2006/0286035 A1
(43) Pub. Date: Dec. 21, 2006

(54) Methods and compositions for modulating the interaction between adiponectin and its receptor

(75) Inventors: Philippe Saudan, Pfungen (CH); Martin Bachmann, Seuzach (CH)

Correspondence Address: STERNE, KESSLER, GOLDSTEIN & FOX PLLC, 1100 NEW YORK AVENUE, N.W., WASHINGTON, DC, 20005, US

(73) Assignee: Cytos Biotechnology AG

(21) Appl. No.: 10/555,058

(22) PCT Filed: Apr. 28, 2004
(86) PCT No.: PCT/EP04/04473

§ 371 Date: Oct. 28, 2005

Related U.S. Application Data
(60) Provisional application No. 60/466,050, filed on Apr. 29, 2003.

(51) Int. Cl. A61K 49/00; A61K 38/17; C07K 14/705; C07H 21/04; C12P 21/06; A61K 39/395

(52) U.S. Cl. 424/9.2; 424/143.1; 435/69.1; 435/320.1; 435/325; 514/12; 530/350; 536/23.5

(57) ABSTRACT

The invention is directed to methods for identifying agents that mimic or modulate the interaction between adiponectin and its receptor. In particular, the invention is directed to methods for mimicking or modulating the adiponectin-T-cadherin interaction in order to treat diseases and disorders associated with a deficiency or overabundance of adiponectin. Such diseases include, e.g., obesity, anorexia nervosa, type I and type II diabetes, coronary artery disease and atherosclerosis. The invention also provides isolated adiponectin-T-cadherin complexes and methods for identifying polypeptides that interact with adiponectin.

### BACKGROUND OF THE INVENTION

1. Field of the Invention

The present invention relates to the fields of medicine and molecular biology. In particular, the invention relates to the modulation of ligand/receptor interactions in the context of treating diseases and conditions such as obesity, anorexia nervosa, type I and type II diabetes, coronary artery disease and atherosclerosis.

2. Related Art

The connection between obesity and the development of several diseases, including type II diabetes, dyslipidemia and arteriosclerosis, is well established. In the past, the adipose tissue was largely viewed as a depot for lipid. Recent observations, however, revealed that adipocytes produce and secrete several bioactive substances such as TNFα, leptin, resistin, plasminogen-activator-protein I, and adiponectin. The adipose tissue is therefore not only the major storage depot for triglycerides, but also an active endocrine organ which senses metabolic signals and secrets hormones that affect whole body energy homeostasis.

Adiponectin (also known as adipocyte complement-related protein (30 kDa) (Acrp30), AdipoQ, apM1, and gelatin-binding protein 28 (GBP28)) is a secreted protein of 30 kD which is produced in adipose tissue. Adiponectin is highly conserved between species. Adiponectin contains a signal sequence, a collagen like domain and a globular domain which was shown to be structurally very similar to the TNF family of proteins. The overall topology of adiponectin bears a close resemblance to the structure of the complement-related protein, C1q. (Berg et al., Trends Endocrinol Metab. 13(2):84-9. (2002)).

Adiponectin forms tightly associated trimers through its globular domain. Higher oligomers are formed through interactions between collagenous triple helices of the adiponectin trimer, resulting in higher order complexes that are found circulating in the plasma (Berg et al., Trends Endocrinol Metab. 13(2):84-9. (2002)).

Mouse studies have demonstrated that the serum adiponectin levels in type II diabetes mouse models are strongly reduced compared to wild-type mice, thereby providing a link between type II diabetes and adiponectin levels in the serum. The results of these studies were corroborated by human and monkey epidemiological studies which showed that adiponectin levels are significantly lower in obese than in lean subjects. Furthermore, diabetic patients were found to have strongly reduced adiponectin levels compared to non-diabetic patients. The levels of adiponectin were shown to be particularly low in diabetic patients who suffer from coronary artery disease (CAD). These serological studies provide a clear link between the adiponectin levels in the serum and the development of type II diabetes and CAD.

Another correlation between low levels of adiponectin and development of type II diabetes was observed in rhesus monkeys which are predisposed to develop insulin resistance. It has been observed that the onset of insulin resistance—a hallmark of type II diabetes—is accompanied by reduced adiponectin levels in the serum. In addition, lean animals with low adiponectin levels showed a much higher degree of insulin resistance than obese animals with high levels of adiponectin. Thus, adiponectin levels rather than leanness are critical for the development of insulin resistance. Adiponectin therefore appears to have a critical role in the development of insulin resistance and type II diabetes.

The importance of adiponectin levels is further emphasized by the observation that thiazolidineduiones (TZDs), which have been shown to improve blood glucose levels in several genetic models of obesity, lead to a marked increase in the adiponectin levels in the serum.

Taken together, these pharmacological and serological data establish a clear link between adiponectin levels in the serum and insulin sensitivity and therefore indicate that the reduced levels of adiponectin found in type II diabetic patients may, at least in part, be responsible for the observed insulin resistance in these subjects.

In addition, recent studies have shown that chronic renal failure, type 1 diabetes and anorexia nervosa are associated with increased adiponectin plasma level and reduced nonoxidative glucose metabolism (Diez et al., 2003 Eur. J. Endocrinol., 148(3), 293-300).

To further understand the key role played by adiponectin in the development of diabetes, several groups have performed in vitro and in vivo studies with different type I and type II mouse models as well as obese wild-type mice.

Injection of recombinant adiponectin in wild-type mice on a high fat diet was shown to substantially improve blood glucose and free fatty acid levels in the serum. Moreover, a significant weight reduction was observed in severely obese mice when administered the globular part of adiponectin over two weeks. Furthermore, the globular part of adiponectin was shown to cause an acute increase of β-oxidation in isolated muscles. Similarly, a clear transient improvement of blood glucose levels was achieved by a single administration of adiponectin to ob/ob (type II diabetes) or NOD mice (type I diabetes). The fact that adiponectin can improve blood glucose levels in both types of diabetes, clearly establishes the insulin sensitizing function of adiponectin. Further, in vitro studies on isolated hepatocytes have demonstrated that adiponectin leads to an improved insulin response. Sub-physiological levels of insulin were shown to be sufficient to shut down hepatic glucose production in the presence of adiponectin.

Besides its insulin sensitizing action on primary hepatocytes, adiponectin has been shown to increase free fatty acid oxidation in differentiated muscle cells and isolated muscles. Furthermore, adiponectin was shown to cause an increased expression of molecules involved in fatty acid transport, combustion and energy dissipation such as CD36, acyl-CoA oxidase (ACO) and uncoupling protein 2 (UCP2). This modulation, in turn, was shown to cause decreased tissue triglycerides content in skeletal muscles.

A recent study showed that adiponectin activates AMP-activated protein kinase (AMPK). (Yamauchi et al. 2002, Nat. Med, 8(11):1288-95) In parallel with the activation of AMPK, adiponectin was shown to stimulate phosphorylation of acetyl coenzyme A carboxylase (ACC), fatty acid oxidation, glucose uptake and lactate production in myocytes, phosphorylation of ACC and reduction of molecules involved in gluconeogenesis in the liver, and reduction of glucose levels in vivo (Yamauchi et al. 2002, Nat. Med, 8(11):1288-95). Finally, adiponectin knock-out mice have been shown to develop diet induced insulin resistance (Maeda et al., 2002, Nat med., 8(7):731-7).

In summary, epidemiological studies, various experiments with diabetes mouse models and the features observed in adiponectin knock-out mice, strongly suggest an important role for adiponectin as a regulator of body energy homeostasis. Therefore, reduced adiponectin levels in the serum may be causally linked with the development of insulin resistance observed in type II diabetes.

Besides its clear effects on the regulation glucose and fatty acid metabolism, several reports suggest a role for adiponectin in the development of coronary artery diseases (CAD) (Matsuda et al., 2002, J Biol Chem. 4; 277(40):37487-91;). This role is believed to be at least partly mediated by adiponectin's action on macrophages and endothelial cells. The observation that decreased adiponectin levels are associated with a higher prevalence of CAD suggested a role for adiponectin in the prevention of arteriosclerosis. Along these lines, several groups have investigated the in vitro action of adiponectin on several cell types known to be involved in the development of CAD.

First, adiponectin was shown to have an effect on monocyte adhesion to endothelium, myeloid differentiation and macrophage cytokine production and phagocytosis. The reduced adhesion of monocytes to the endothelium can at least partly be attributed to adiponectin's ability to suppress TNFα-induced vascular cell adhesion molecule I (VCAM-I), endothelial leukocyte adhesion molecule-1 (E-selectin), and intracellular adhesion molecule I (ICAM-1) on human aortic endothelial cells.

Furthermore, adiponectin was shown to decrease lipid accumulation in human monocyte-derived macrophages and to suppress macrophage-to-foam cell transformation.

The effects of adiponectin are related to atherosclerotic plaque formation. The in vitro data suggest an anti-arterogenic property of adiponectin. Hypoadiponectinemia, therefore, might be associated with a higher incidence of vascular diseases.

Moreover, adiponectin's effect on neointimal thickening after artery injury, a hallmark of arteriosclerosis, has been studied in vivo. In one study, adiponectin-deficient mice were compared to wild-type mice. Adiponectin-deficient mice showed severe neointimal thickening and increase proliferation of vascular smooth muscle cells in mechanically injured arteries, suggesting that adiponectin prevents this effect under normal circumstances. This conclusion is further corroborated by the observation that neointimal thickening in the surgical model was strongly reduced by the administration of a recombinant adenovirus expressing adiponectin (Matsuda et al, 2002, J Biol Chem. 4; 277(40):37487-91).

Further studies on cultivated smooth muscle cells revealed that adiponectin inhibited DNA synthesis induced by growth factors such as PDGF, HB-EGF, bEGF and EGF, and cell proliferation and migration induced by HB-EGF. Finally, over-expression of the globular domain of adiponectin in an apoE-deficient background (an established mouse model for the development of arteriosclerosis) showed a clear improvement of arteriosclerosis (Yamauchi et al., J. Biol. Chem. 278:2461-2468 (2003)). The in vivo results further indicate a protective role of adiponectin against the development of CAD.

In summary, several serological studies and in vivo and in vitro studies highlight adiponectin's important role as a regulator of lipid metabolism and insulin sensitivity as well as an anti-artherogenic factor.

Heretofore, very little has been known about the signaling mechanisms which are responsible for the actions of adiponectin. Accordingly, in order to effectively treat diseases and conditions associated with a deficiency or over-abundance of adiponectin, there is a need in the art for methods and compositions that modulate or mimic the actions of adiponectin.

### SUMMARY OF THE INVENTION

The present invention satisfies the aforementioned need in the art. The invention provides methods for identifying agents that modulate or mimic the actions of adiponectin. The invention is based on the surprising discovery that T-cadherin is a receptor for adiponectin. Therefore, the invention provides methods and agents that modulate or mimic the interaction between adiponectin and T-cadherin. The invention also provides isolated adiponectin-T-cadherin complexes and methods for identifying polypeptides that interact with adiponectin.

T-cadherin (also known as truncated or H cadherin, or cadherin 13) is a member of the cadherin superfamily (Angst, B. D. et al., J. Cell Sci. 114:629-641 (2001)). T-cadherin is a GPI-anchored protein that is believed to be involved in cell signaling (Doyle et al., J. Biol. Chem. 273:6937-6943 (1998), Philippova et al., FEBS Lett. 429:207-210 (1998)). Thus, it is likely that at least certain actions of adiponectin are mediated through its interaction with T-cadherin. By modulating the interaction between adiponectin and T-cadherin, or by providing agents that mimic an action of adiponectin, the cellular and physiological actions of adiponectin can be deliberately controlled in the context of treating diseases and conditions that are characterized by a deficiency or overabundance of adiponectin. Such modulation can be achieved, for example, by blocking the interaction between adiponectin and T-cadherin, by enhancing the interaction between adiponectin and T-cadherin, and by providing agents that mimic the action of adiponectin.

Agents that mimic an activity of adiponectin are useful, e.g., in the treatment of diseases and conditions that are associated with a deficiency of adiponectin. Such agents can be administered to patients afflicted with diseases and conditions such as, e.g., obesity, type I and type II diabetes, coronary artery disease, anorexia nervosa and atherosclerosis in order to alleviate or treat such diseases and conditions.

In view of the finding that T-cadherin is a receptor for adiponectin, it follows that molecules and compositions that interact with T-cadherin are likely candidates for agents that mimic an activity of adiponectin. In certain aspects of the invention, therefore, methods are provided for determining whether agents, e.g., agents that interact with T-cadherin, mimic an activity of adiponectin.

According to one aspect of the invention, methods are provided for determining whether an agent mimics an activity of adiponectin. For example, the invention includes methods for screening multiple agents, such as antibodies and other molecules or compounds, for the ability to mimic an action of adiponectin. The methods according to this aspect of the invention, in certain embodiments, may comprise: (a) obtaining a test agent (such as, e.g., an antibody) that interacts with T-cadherin; (b) administering the test agent to a first animal; (c) measuring a physiological parameter in the first animal after administration of the agent; (d) measuring the physiological parameter in one or more control subjects; and (e) comparing the physiological parameter in the first animal after administration of the agent to the physiological parameter in one or more control subjects.

The physiological parameter can be any parameter that is known to be altered (e.g., increased or decreased) following the administration of adiponectin to an animal. Such parameters are known in the art. Exemplary parameters are described elsewhere herein. An agent is identified as one that mimics an action of adiponectin if the physiological parameter measured in the first animal after administration of the agent is altered (e.g., higher or lower, depending on the physiological parameter measured) as compared to the physiological parameter measured in one or more of the control subjects.

The methods according to this aspect of the invention also comprise: (a) obtaining an agent (such as, e.g., an antibody) that interacts with T-cadherin; (b) contacting a first cell with the agent; (c) measuring a cellular parameter in the first cell after contacting the first cell with the agent; (d) measuring the cellular parameter in one or more control cells; and (e) comparing the cellular parameter in the first cell after contacting the first cell with the agent to the cellular parameter in one or more of the control cells. The cellular parameter can be any parameter that is known to be altered (e.g., increased or decreased) following contacting a cell with adiponectin. Such parameters are known in the art. An agent is identified as one that mimics an action of adiponectin if the cellular parameter measured in the first cell after contacting the first cell with the agent is altered (e.g., higher or lower, depending on the cellular parameter measured) as compared to the cellular parameter measured in one or more of the control cells.

In other aspects, the invention provides methods for determining whether a test agent mimics an action of adiponectin. Such methods may comprise: (a) contacting a first cell with a test agent, wherein the first cell expresses T-cadherin; (b) contacting a second cell with the test agent, wherein the second cell does not express T-cadherin; (c) measuring a cellular parameter in the first and second cells after contacting the first and second cells with the test agent; and (d) comparing the cellular parameter in the first cell to the cellular parameter in the second cell after contacting the first and second cells with the test agent. A difference (e.g., an increase or decrease, depending on the cellular parameter measured) in the cellular parameter in the first cell as compared to the cellular parameter in the second cell, after contacting the first and second cells with the test agent, will identify the test agent as one that mimics an action of adiponectin.

According to another aspect of the invention, methods are provided for determining whether a test agent inhibits or enhances the interaction between adiponectin and T-cadherin. The methods according to this aspect of the invention comprise any known assay that enables one to measure the interaction between two agents, such as, e.g., assays that measure protein-protein interactions. Many such methods are known in the art and can be adapted to measure the interactions between adiponectin and T-cadherin. (Comb. Chem. High Throughput Screen. 1998 December; 1(4):171-183. Review).

In one exemplary embodiment according to this aspect of the invention, methods are provided comprising: (a) providing a test mixture comprising: (i) adiponectin or a fragment thereof and (ii) T-cadherin or a fragment thereof, one of them being fused to a detectable marker or enzyme and the other being immobilized on a suitable carrier, and (iii) a test agent; (b) providing a control mixture comprising: (i) adiponectin or a fragment thereof and (ii) T-cadherin or a fragment thereof, one of them being fused to a detectable marker or enzyme and the other being immobilized on a suitable carrier; (c) removing unbound adiponectin and T-cadherin from the test mixture and from the control mixture; and (d) measuring the signal produced by the marker or enzyme in the test mixture and in the control mixture.

In another exemplary embodiment, method are provided comprising: (a) providing a test mixture comprising: (i) adiponectin or a fragment thereof and (ii) T-cadherin or a fragment thereof, one of them being fused to a detectable marker or enzyme and the other being expressed on the surface of a cell, and (iii) a test agent; (b) providing a control mixture comprising: (i) adiponectin or a fragment thereof and (ii) T-cadherin or a fragment thereof, one of them being fused to a detectable marker or enzyme and the other being expressed on the surface of a cell; (c) removing unbound adiponectin and T-cadherin from the test mixture and from the control mixture; and (d) measuring the signal produced by the marker or enzyme in the test mixture and in the control mixture.

In either of the aforementioned exemplary embodiments, a decrease in the signal produced by the marker or enzyme in the test mixture as compared to the signal produced by the marker or enzyme in the control mixture indicates the ability of the test agent to inhibit the interaction between adiponectin and T-cadherin. Alternatively, an increase in the signal produced by the marker or enzyme in the test mixture as compared to the signal produced by the marker or enzyme in the control mixture indicates the ability of the test agent to enhance the interaction between adiponectin and T-cadherin.

According to another aspect of the invention, methods are provided for identifying a polypeptide that interacts with adiponectin. For example, the invention includes methods for screening libraries (e.g., genomic DNA libraries or cDNA libraries, etc.) for clones that express a polypeptide that interacts with adiponectin. In certain embodiments according to this aspect of the invention, methods are provided comprising: (a) obtaining a population of cells, wherein the population comprises two or more cells that express different candidate polypeptides on their respective surfaces; (b) contacting the population of cells with a bait polypeptide. The bait polypeptide can be, e.g., adiponectin, a fragment of adiponectin, adiponectin fused to a detectable marker or enzyme, or a fragment of adiponectin fused to a detectable marker or enzyme. The methods further comprise: (c) separating cells which have the bait polypeptide bound to them from cells that do not have the bait polypeptide bound to them; and (d) identifying the candidate polypeptide that is expressed on the surface of the cells which have the bait polypeptide bound to them. In certain embodiments, the candidate polypeptide that is expressed on the surface of the cells which have the bait polypeptide bound to them, will be a polypeptide that is expressed from an expression vector (e.g., an expression vector from a library), in the cells. Thus, its identity can easily be determined by cloning the nucleotide sequence included within the expression vector.

The invention also includes isolated receptor-ligand complexes comprising: (a) adiponectin or a fragment or variant thereof; and (b) T-cadherin or a fragment or variant thereof. For example, the invention includes isolated adiponectin-T-cadherin complexes, wherein adiponectin or a fragment or variant thereof is physically attached to or in contact with T-cadherin a fragment or variant thereof.

Also included within the invention are methods for producing a pharmaceutical composition useful for treating a disease or condition related to a deficiency or over-abundance of adiponectin such as, e.g., obesity, anorexia nervosa, coronary artery disease, type I and type II diabetes, etc. The methods according to this aspect of the invention comprise: (a) obtaining an agent or substance that mimics an action of adiponectin, or that inhibits or enhances the interaction between adiponectin and T-cadherin; and (b) mixing the molecule or substance with a pharmaceutically acceptable carrier or excipient. The molecule or substance may be obtained using any of the methods of the invention.

The invention also includes methods for treating or preventing a disease or condition related to a deficiency or over-abundance of adiponectin such as, e.g., obesity, anorexia nervosa, coronary artery disease, type I and type II diabetes, etc. The methods according to this aspect of the invention comprise administering to a subject a therapeutically effective amount of an agent or substance that mimics an action of adiponectin, or that inhibits or enhances the interaction between adiponectin and T-cadherin. The agent or substance may be obtained using any of the methods of the invention.

Also provided are antibodies and other agents that are specifically reactive against the receptor-ligand complex (e.g., a receptor-ligand complex (e.g., a T-cadherin-adiponectin complex)) of the invention or against T-cadherin.

The invention further provides a use of a receptor-ligand complex of the invention for the preparation of a medicament for treating and/or preventing diseases or conditions including, preferably selected from, hypoadiponectinemia, obesity, anorexia nervosa, coronary artery disease, type I diabetes, type II diabetes, and atherosclerosis.

Also included in the invention are nucleic acid vectors comprising the receptor-ligand complex of the invention, as well as host cells comprising the vectors or nucleic acids, and transgenic, knockout or genetically modified animals (other than humans, in particular mice), comprising manipulated nucleic acids of the invention or lacking the endogenous sequence.

In a further aspect, a kit comprising a receptor-ligand complex of the invention is provided.

The invention further provides a method of diagnosing or prognosing diseases or conditions such as, e.g., hypoadiponectinemia, obesity, anorexia nervosa, coronary artery disease, type I diabetes, type II diabetes, and atherosclerosis. Such methods comprise: (i) obtaining a sample from an individual; (ii) analyzing said sample for the presence of a receptor-ligand complex of the invention (e.g., a T-cadherin-adiponectin complex); and (iii) comparing the levels of receptor-ligand complex in the test sample to the level of said complex in healthy tissue; wherein as decrease in receptor-ligand complex concentration in the test sample compared to that of healthy tissue indicates that the individual is at risk for a disease such as hypoadiponectinemia, obesity, anorexia nervosa, coronary artery disease, type I diabetes, type II diabetes, and atherosclerosis.

The invention also provides a kit suitable for use in the diagnostic or prognostic methods of the invention. Such kits comprise reagents useful for carrying out these methods, for example, antibodies from one or more species specific for the receptor-ligand complex (e.g., a receptor-ligand complex (e.g., a T-cadherin-adiponectin complex)) or for T-cadherin. Secondary antibodies that recognize either or both such primary anti-T-cadherin antibodies can also be included for the purpose of recognition and detection of primary antibody binding to a sample. Such secondary antibodies can be labeled for detection e.g. with fluorophores, enzymes, radioactive labels or otherwise. Other detection labels will be evident to those skilled in the art. Alternatively, the primary anti-T-cadherin antibodies can be labeled for direction detection.

### BRIEF DESCRIPTION OF THE FIGURES

FIG. 1: Validation of adiponectin T-cadherin binding in transiently transfected cells. 293-EBNA cells were transfected with pGF-T-cadherin. Two days after transfection the cells were stained either with Acrp30-FLAG-C, followed by incubation with mouse anti-FLAG, and Cy5-coupled anti mouse antibodies or with secondary reagents only. The cells were then analyzed by flow cytometry. Bait binding (Cy5 fluorescence) is shown on the Y-axis, whereas GFP expression is shown on the X-axis. In the sample which was stained with Acrp30-FLAG-C, only the GFP positive population is shifted, whereas no shift was observed in the sample stained with the secondary reagents only. These data show that adiponectin is specifically interacting with T-cadherin.

FIG. 2: Determination of the Kd of the adiponectin T-cadherin interaction. 293-EBNA cells were transfected with pGF-T-cadherin. After two days the cells were harvested and stained with decreasing amounts of Acrp30-FLAG-C (50, 25, 12.5, 6.3, 3.1, 1.6, 0.8, 0.4 and 0 μg/ml) as described in example 6. To evaluate the dissociation constant, the geometric mean fluorescence (GMFl) of the GFP positive population was determined using FACS Winmdi software. The GMFl was then normalized as follows: (GMFL of the sample minus GMFl of samples stained with secondary reagents only) divided by maximal GMFL. The protein concentration is shown on the X axis and the normalised GMFL is shown on the Y axis. The Kd was determined by division of the concentration required for half maximal binding (2.2 μg/ml) by the molecular weight of the protein (26546 g/mol). The Kd was determined as being 83 nM.

FIG. 3: An anti T-cadherin antibody reduces blood glucose levels. Male FVB mice were starved for 2 h and injected with PBS, 100 μg rabbit gamma globulin or 100 μg rabbit anti T-cadherin antibody (Santa Cruz, sc-7940). The blood glucose levels were measured with a trinder assay at the indicated time points and all values were normalized to the baseline glucose levels (glucose levels before the injection=0 h). During the whole experiment the animals were not allowed access to food. Error bars represent the standard error of the means of 5 animals.

### DETAILED DESCRIPTION OF THE INVENTION

Definitions

As used herein, the term “T-cadherin” refers to any cadherin including T-cadherin (truncated), H cadherin (heart), cadherin 13, the mature 105 kDa T-cadherin, the partially processed 130 kDa precursor of T-cadherin, or to any protein that is encoded by any of the nucleotide sequences selected from the sequences set forth in SEQ ID NO:21, SEQ ID NO:23, SEQ ID NO:25, SEQ ID NO:27, SEQ ID NO:29, SEQ ID NO:31, SEQ ID NO:33 to SEQ ID NO:47, SEQ ID NO:49, SEQ ID NO:51, and SEQ ID NO:53, or to sequences with at least 80% identity, preferably with at least 90% identity, more preferably with at least 95% identity, even more preferably with at least 98% identity, most preferably with at least 99% identity to any of the above mentioned sequences, or to fragments thereof. The terms “T-cadherin”, “H-Cadherin”, and “Cadherin 13”, as used herein, may be used interchangeably.

As used herein, an agent that “mimics an action of adiponectin” or that is “capable of mimicking an action of adiponectin” refers to any substance, molecule or compound, including antibodies, that may act in the same, similar or functionally the same way as adiponectin. (see Yamauchi et al. 2002, Nat. Med, 8(11):1288-95).

An agent that mimics an action of adiponectin may, for example, regulate energy homeostastis and glucose and lipid metabolism, may stimulate phosphorylation and activation of AMPK (5′-AMP-activated protein kinase) and of ACC (acety coenzyme A carboxylase), fatty-acid oxidation, glucose uptake and lactate production in myocytes, and reduction of molecules involved in gluconeogenesis, and may stimulate reduction of blood glucose concentration, blood free fatty acid concentration, blood triglyceride concentration, or may reduced neointimal thickening after artery injury.

As used herein, the phrase “response that corresponds to a response caused by an action of adiponectin”, or the term “adiponectin-like response” refers to a response of e.g. an animal, a cell, a tissue, or a sample, that is the same, similar, or functionally the same response as a response of said cell, tissue or sample that is caused by adiponectin. For example, a response caused by an antibody or other agent of the invention may be the same, similar, or functionally the same response as the response caused by adiponectin (see Yamauchi et al. 2002, Nat. Med, 8(11):1288-95), such as the stimulation of the phosphorylation and activation of AMPK (5′-AMP-activated protein kinase) and of ACC (acety coenzyme A carboxylase), fatty-acid oxidation, glucose uptake and lactate production in myocytes, and reduction of molecules involved in gluconeogenesis, and the stimulation of the reduction of blood glucose concentration, blood free fatty acid concentration, blood triglyceride concentration, or may reduced neointimal thickening after artery injury.

As used herein, the expression, “methods for determining whether an agent (molecule, compound, antibody, polypeptide, etc.) mimics an activity of adiponectin” is equivalent to the expressions “methods for identifying agents that mimic an activity of adiponectin,” “methods for screening agents that mimic an activity of adiponectin,” “methods for identifying agents with adiponectin-like activity,” and the like.

As used herein, the term “purified,” used in reference to a given agent, means that the concentration of the agent being purified has been increased relative to substances associated with it in its natural environment. Naturally associated substances include polypeptides, nucleic acids, lipids and sugars but generally do not include water, buffers, and reagents added to maintain the integrity or to facilitate the purification of the molecule. For example, even if mRNA is diluted with an aqueous solvent during oligo dT column chromatography, mRNA molecules are purified by this chromatography if naturally associated nucleic acids and other biological molecules do not bind to the column and are separated from the subject mRNA molecules.

As used herein, the term “isolated” used in reference to an agent means that the agent has been removed from its native environment. For example, a polynucleotide or a polypeptide naturally present in a living animal is not “isolated,” but the same polynucleotide or polypeptide separated from the coexisting materials of its natural state is “isolated.” Further, recombinant DNA molecules contained in a vector are considered isolated for the purposes of the present invention. Isolated RNA molecules include in vivo or in vitro RNA replication products of DNA and RNA molecules. Isolated nucleic acid molecules further include synthetically produced molecules. Additionally, vector molecules contained in recombinant host cells are also isolated. Thus, not all “isolated” agents need to be “purified.”

As used herein, the term “individual” refers to multicellular organisms and includes both plants and animals. Preferred multicellular organisms are animals, more preferred are vertebrates, even more preferred are mammals, and most preferred are humans.

As used herein, the term “vector” refers to a polynucleotide construct, typically a plasmid or a virus, used to transmit genetic material to a host cell. Preferably, the term “vector” as used herein refers to a molecule such as a plasmid, and even more preferably to a circular plasmid. A vector as used herein may be composed of either DNA or RNA. Preferably, a vector as used herein is composed of DNA.

As used herein, a nucleic acid, or fragment thereof, that hybridizes with other nucleic acids. (e.g., a nucleic acid encoding T-cadherin, adiponectin, or the receptor-ligand complex of the invention), for example, one of the nucleic acids having a sequence of SEQ ID NO: 6, SEQ ID NO:21, SEQ ID NO:23, SEQ ID NO:25, SEQ ID NO:27, SEQ ID NO:29, SEQ ID NO:31, SEQ ID NO:33 to SEQ ID NO:47, SEQ ID NO:49, SEQ ID NO:51, or SEQ ID NO:53, indicates a nucleic acid sequence that hybridizes under stringent conditions with a counterpart of a nucleic acid having the features of a nucleotide sequence with at least 80% identity to any of the above sequences; a nucleic acid that hybridizes to one of the above mentioned nucleotide sequences; a nucleotide sequence complementary to any of the above mentioned nucleotide sequences; or a fragment of any of the above mentioned nucleotide sequences, that hybridizes to one of the above mentioned nucleotide sequences. For example, hybridizing may be performed at 68° C. in 2×SSC or according to the protocol of the dioxygenine-labeling-kits of the Boehringer (Mannheim) company. A further example of stringent hybridizing conditions is, for example, the incubation at 65° C. overnight in 7% SDS, 1% BSA, 1 mM EDTA, 250 mM sodium phosphate buffer (pH 7.2) and subsequent washing at 65° C. with 2×SSC; 0.1% SDS.

The term “percent identity” as known to the skilled artisan and used herein indicates the degree of relatedness among 2 or more nucleic acid molecules that is determined by agreement among the sequences. The percentage of “identity” is the result of the percentage of identical regions in 2 or more sequences while taking into consideration the gaps and other sequence peculiarities.

The identity of related nucleic acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Computer programs for determining the identity among two sequences comprise, but are not limited to, the GCG-program package, including GAP (Devereux et al., Nucleic Acids Research 12 (12):387 (1984); Genetics Computer Group University of Wisconsin, Madison, (WI)); BLASTP, BLASTN, and FASTA (Altschul et al., J. Molec. Biol 215:403/410(1990)). The BLAST X program can be obtained from the National Center for Biotechnology Information (NCBI) and from other sources (BLAST handbook, Altschul et al., NCB NLM NIH Bethesda, Md. 20894). Also, the well-known Smith-Waterman algorithm can be used for determining identity.

Preferred parameters for sequence comparison comprise the following:

| Algorithm | Needleman and Wunsch, J. Mol. Biol. 48: 443-453 |
|---|---|
| | (1970) |
| Comparison matrix | Matches = +10, mismatch 0 |
| Gap penalty: | 50 |
| Gap length penalty: | 3 |

The gap program is also suited to be used with the above-mentioned parameters. The above-mentioned parameters are standard parameters (default) for nucleic acid comparisons. Further exemplary algorithms, gap opening penalties, gap extension penalties, comparison matrix, including those in the program handbook, Wisconsin-package, version 9, September 1997, can also be used. The selection depends on the comparison to be done and further, whether a comparison among sequence pairs, for which GAP or Best Fit is preferred, or whether a comparison among a sequence and a large sequence databank, for which FASTA or BLAST is preferred, is desired.

The nucleic acid molecules according to the invention may be prepared synthetically by methods well-known to the skilled person, but also may be isolated from suitable DNA libraries and other publicly-available sources of nucleic acids and subsequently may optionally be mutated. The preparation of such libraries or mutations is well-known to the person skilled in the art.

As used herein, a “fragment” of a reference polypeptide (e.g., a fragment of adiponectin or a fragment of T-cadherin) includes, e.g., a polypeptide having at least one less amino acid than the reference polypeptide. Fragments of a reference polypeptide can be obtained, e.g., by engineering a nucleic acid molecule to express a polypeptide having at least one less amino acid than the reference polypeptide. Alternatively, fragments of a reference polypeptide can be obtained by proteolytically cleaving the reference polypeptide or by artificially synthesizing a polypeptide has at least one less amino acid than the reference polypeptide. A “fragment” of a reference polypeptide may differ from the reference polypeptide by lacking one or more amino acids at one or both of its termini, or it may lack one or more internal (non-terminal) amino acids. Fragments and derivatives and variants of Adiponectin and T-cadherin, may contain minor modifications of the sequences of Adiponectin and T-cadherin which do not destroy its immunoreactivity. Limited modification may be made without destroying the biological function of T-cadherin and Adiponectin, and only a portion of the entire primary structure may be required to effect activity. Such minor modifications may result in proteins which have substantially equivalent or enhanced function. Preferred fragments are fragments which are recognized by an antibody that is also capable of recognizing the full length Adiponectin or the full length T-cadherin. Exemplary fragments of Adiponectin may comprise, or alternatively essentially consist of, or alternatively consist of the globular domain or the collagen domain of Adiponectin. Exemplary fragments of T-cadherin may comprise, or alternatively essentially consist of, or alternatively consist of one or more of the five cadherin domains of T-cadherin (SEQ ID NO: 55, SEQ ID NO: 56, SEQ ID NO: 57, SEQ ID NO: 58, SEQ ID NO: 59), preferably the cadherin domain 1 of T-cadherin (SEQ ID NO: 55).

As used herein, a “variant” of a polypeptide sequence is intended to include natural and man-made allelic polypeptide sequences possessing conservative or non-conservative amino acid substitutions, deletions or insertions. Also included are polypeptides containing one or more amino acid analogs, one or more non-classical amino acids, and/or one or more post-translational modifications. Non-classical amino acids include, but are not limited to, to the D-isomers of the common amino acids, 2,4-diaminobutyric acid, a-amino isobutyric acid, 4-aminobutyric acid, Abu, 2 amino butyric acid, g Abu, e Ahx, 6 amino hexanoic acid, Aib, 2 amino isobutyric acid, 3 amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary). Post-translational modifications include, but are not limited to glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc. Variants also include polypeptides having N linked or O linked carbohydrate chains, chemical moieties, chemical modifications of N linked or O linked carbohydrate chains, and addition or deletion of an N terminal methionine residue as a result of prokaryotic host cell expression. Variant polypeptides may also be modified with a detectable label, such as an enzymatic, fluorescent, isotopic or affinity label to allow for detection and isolation of the protein.

When the terms “one,” “a,” or “an” are used in this disclosure, they mean “at least one” or “one or more,” unless otherwise indicated.

### EMBODIMENTS OF THE INVENTION

According to one aspect of the present invention, methods are provided for identifying agents that mimic an action of adiponectin.

The invention includes methods for determining whether a test agent mimics an action of adiponectin, said method comprising: (a) obtaining a test agent; (b) administering the test agent to a first animal; (c) measuring a physiological parameter in the first animal after administration of the agent; and (d) comparing the physiological parameter in the first animal after administration of the agent to the physiological parameter in a control subject.

The invention also includes methods for determining whether a test agent mimics an action of adiponectin, said method comprising: (a) obtaining a test agent; (b) contacting a first cell with the agent; (c) measuring a cellular parameter in the first cell after contacting the first cell with the agent; and (d) comparing the cellular parameter in the first cell after contacting the first cell with the agent to the cellular parameter in a control cell.

As used herein, the expression “test agent” is intended to mean a molecule, compound or other substance that is tested for the ability to mimic an action of adiponectin. A test agent can be any compound, substance or composition. The test agent may be selected from the group consisting of peptides, peptide analogs, nucleic acids, nucleic acid analogs, hormones, antigens, synthetic or naturally occurring drugs, opiates, dopamine, PNAs, serotonin, catecholamines, thrombin, acetylcholone, prostaglandins, organic molecules, pheromones, adenosine, sucrose, glucose, lactose and galactose. Exemplary test agents include antibodies and non-protein compositions. In certain embodiments of the invention, the test agent interacts with T-cadherin or with fragments or derivatives of T-cadherin.

The agent that interacts with T-cadherin may be, e.g., a polypeptide or other small chemical compound that exhibits physical interaction with T-cadherin. Any known methods for assaying the physical interaction of two molecules with one another can be used to determine if an agent is one that interacts with T-cadherin. Methods for obtaining agents that interact with specific polypeptides are known in the art and include, e.g., screening libraries of agents for those that bind to the polypeptides of interest.

The expression “agent that interacts with T-cadherin” is intended to mean an agent that interacts with wild-type full length T-cadherin or an agent that interacts with mutant or variant forms of T-cadherin including, but not limited to, fragments of T-cadherin, T-cadherin variants with one or more altered amino acid residues as compared to the wild-type molecule, T-cadherin variants having one or more post-translational modifications, and fusion proteins in which all or part of T-cadherin is fused to one or more additional polypeptides or fragments thereof.

The agent that interacts with T-cadherin may be, e.g., an antibody. In certain embodiments, the antibody is a monoclonal antibody that interacts with T-cadherin.

In certain aspects of the invention, the test agent is an agent that interacts with T-cadherin. The invention, in certain aspects, involves determining whether an agent that interacts with T-cadherin mimics an action of adiponectin. Thus, invention involves obtaining a test agent that interacts with T-cadherin. In order to obtain a test agent that interacts with T-cadherin, any method can be used that allows one to assess or monitor the interaction between two molecules or compounds. In certain instances, in order to obtain a test agent that interacts with T-cadherin, libraries of compounds or molecules are initially screened to identify those that interact with T-cadherin. Once identified, compounds or molecules from the library that interact with T-cadherin can be used in the methods of the invention to determine if such compounds or molecules mimic an action of adiponectin.

In other aspects of the invention, the test agent that is tested for its ability to mimic an action of adiponectin does not necessarily interact with T-cadherin. Persons skilled in the art will appreciate the methods available for obtaining test agents for use with the invention.

Once obtained, the test agent can be administered to a first animal. The agent may be administered by itself or in combination with other substances such as buffers, diluents, or pharmacologically-acceptable carriers. (Remington's Pharmaceutical Sciences, Mack Publishing Co. (1990)). The agent can be administered by any known route of administration including, e.g., injection, administration through the skin, and oral administration.

After the agent is administered to a first animal, a physiological parameter is measured in said first animal. The physiological parameter that is measured is any parameter that is influenced, e.g., increased, enhanced, stimulated, decreased, attenuated, suppressed, etc., by the administration of adiponectin. Such parameters are known in the art. Exemplary physiological parameters include but are not limited to, e.g., blood glucose concentration, blood free fatty acid concentration, blood triglyceride concentration, neointimal thickening and lesions associated with coronary artery disease (e.g., aortic valve lesions). Other physiological parameters include, e.g., fatty acid oxidation in muscle cells, hepatic glucose output, adiponectin concentration, weight gain, weight loss, and insulin response. Methods for measuring such physiological parameters are known in the art. (See, e.g., Berg et al., Trends Endocrinol. & Metab. 13:84-89 (2002); Fruebis et al., Proc. Natl. Acad. Sci. USA 98:2005-2010 (2001)).

The physiological parameter can be measured immediately after administration of the agent, or at any time following the administration. The physiological parameter can be measured multiple times following the administration, e.g., at regular time intervals.

The methods according to this aspect of the invention may also comprise measuring the physiological parameter in one or more control subjects. The physiological parameter measured in the control subject can be compared to the physiological parameter measured in the first animal after administration of the agent. Preferred control subjects include: (i) the first animal prior to the administration of the agent; and (ii) a second animal to which the agent has not been administered. Thus, as used herein, the “control subject,” in certain instances, may be the same animal as the animal to which the molecule is administered but at an earlier point in time, i.e., before the agent is administered.

The methods of the invention also comprise comparing the physiological parameter in the first animal after administration of the agent to the physiological parameter in the control subject. A difference in the physiological parameter in the first animal after administration of the agent relative to the physiological parameter in the control subject will indicate whether the agent that interacts with T-cadherin mimics an action of adiponectin.

For instance, an agent will be identified as one that mimics an action of adiponectin if: (i) the physiological parameter that is measured is one that is known to increase upon the administration of adiponectin, and (ii) the physiological parameter in the first animal after administration of the agent is greater than the physiological parameter in the control subject. For example, if the physiological parameter that is measured is fatty acid oxidation in muscle cells, then an agent will be identified as one that mimics an action of adiponectin if the level of fatty acid oxidation in the first animal after administration of the agent is greater than the level of fatty acid oxidation in the control subject.

An agent will also be identified as one that mimics an action of adiponectin if: (i) the physiological parameter that is measured is one that is known to decrease upon the administration of adiponectin, and (ii) the physiological parameter in the first animal after administration of the agent is less than the physiological parameter in the control subject. For example, if the physiological parameter that is measured is blood glucose concentration, blood free fatty acid concentration or blood triglyceride concentration, then an agent will be identified as one that mimics an action of adiponectin if the physiological parameter in the first animal after administration of the molecule is less than the physiological parameter in the control subject.

The animals that are used in the methods of the invention, e.g., the first and the second animals, may be any animal for which a physiological parameter relating to an action of adiponectin can be measured following the administration of an agent. Preferred animals are vertebrates, including, e.g., mammals such as mice, rats, rabbits, pigs, cows, sheep and humans. In certain instances, the animal is a transgenic or mutant animal. The animal may, in certain instances, be a genetically modified animal that exhibits a higher or lower level of a physiological parameter as compared to a corresponding wild-type animal. Exemplary genetically modified animals that can be used in the context of the present invention include, e.g., ob/ob mice, NOD mice and ApoE-deficient mice.

The invention also includes methods for determining whether a test agent mimics an action of adiponectin, said method comprising: (a) obtaining a test agent; (b) contacting a first cell with the agent; (c) measuring a cellular parameter in the first cell after contacting the first cell with the agent; and (d) comparing the cellular parameter in the first cell after contacting the first cell with the agent to the cellular parameter in a control cell.

The test agent may be, e.g., an agent that interacts with T-cadherin. The agent that interacts with T-cadherin may be, e.g., a polypeptide or other small chemical compound that exhibits physical interaction with T-cadherin. Any known methods for assaying the physical interaction of two molecules with one another can be used to determine if an agent is one that interacts with T-cadherin. Methods for obtaining molecules that interact with specific polypeptides are known in the art and include, e.g., screening libraries of agents for those that bind to the polypeptides of interest.

The agent that interacts with T-cadherin may be, e.g., an antibody. In certain embodiments, the antibody is a monoclonal antibody that interacts with T-cadherin.

Methods for generating antibodies, e.g., monoclonal antibodies, that interact with a particular protein or a fragment of the protein are well known in the art. For example, to generate monoclonal antibodies that recognize T-cadherin or a fragment thereof, animals, preferably mice or rats and more preferably mice with a humanized B cell repertoire can be injected with T-cadherin or a fragment of T-cadherin. The fragment of T-cadherin may be any portion of T-cadherin. A preferred fragment is one comprising the extracelluar part of T-cadherin. T-cadherin or a fragment of T-cadherin may be coupled to a carrier, preferably protein carrier, prior to injecting the animal. Alternatively, the animal may be injected with DNA encoding T-cadherin or a fragment thereof. The DNA molecule is preferably linked to a T helper cell epitope. Monoclonal antibodies are generated thereafter using standard methods (see e.g. Chapter 6, Antibodies A Laboratory Manual, Cold Spring Harbor Laboratory Press, 1988).

T-cadherin antibodies have been described, e.g., in Ivanov et al., Histochem. Cell. Biol. 115:231-242 (2001) and in U.S. Pat. No. 5,863,804.

Once obtained, the test agent is contacted with a first cell. For example, to contact an agent with the first cell, the agent can be added to a container that contains the first cell. In certain embodiments, the container is a test tube, petri dish, vial, bottle, or other similar vessel, and the first cell is within the container along with a suitable liquid or solid medium. the agent is contacted with the cell by, e.g., adding the molecule to the liquid or solid medium. Alternatively, the first cell can be added directly to the molecule or to a composition (e.g., a solution) comprising the molecule.

After the agent is contacted with a first cell, a cellular parameter is measured in the cell. The cellular parameter that is measured is any parameter that is influenced, e.g., increased, enhanced, stimulated, decreased, attenuated, suppressed, etc., by adiponectin. Exemplary cellular parameters include, e.g., fatty acid oxidation, glucose uptake or output, lactate production, 5′-AMP-activated protein kinase (AMPK) phosphorylation, acetyl coenzyme A carboxylase (ACC) phosphorylation, IRS-1-mediated PI-3-kinase activity, smooth muscle cell proliferation and/or migration, NF-κB signaling, cAMP production and TNF-α-induced expression of endothelial adhesion molecules (e.g., vascular cell adhesion molecule-1 (VCAM-1), endothelial-leukocyte adhesion molecule-1 (E-selectin), and intracellular adhesion molecule-1 (ICAM-1)). Other cellular parameters include, cellular adhesion (e.g., monocyte adhesion to endothelium), myeloid differentiation, macrophage cytokine production, phagocytosis, lipid accumulation, and uptake of acetylated low-density lipoprotein particles. (See Berg et al., Trends Endocrinol. & Metab. 13:84-89 (2002)). Methods for measuring such cellular parameters are known in the art. (See, e.g., Berg et al., Trends Endocrinol. & Metab. 13:84-89 (2002); Yamauchi et al., Nat. Med. 8:1288-1295 (2002)).

The cellular parameter can be measured immediately after contacting the cell with the agent, or at any time thereafter. The cellular parameter can be measured multiple times after the cell is contacted with the agent, e.g., at regular time intervals.

The methods of the invention may, in certain instances, also comprise measuring the cellular parameter in one or more control cells. The cellular parameter measured in the control cell can be compared to the cellular parameter measured in the first cell after contacting the first cell with the agent. Preferred control cells include: (i) the first cell prior to contacting the first cell with the agent; and (ii) a second cell that has not been in contact with the agent. In certain embodiments of the invention, the first cell is a cell that expresses T-cadherin. When the first cell expresses T-cadherin, the control cell may also be (iii) a second cell that does not express T-cadherin. As used herein, a “control cell,” in certain instances, may be the same cell as the cell with which the agent is contacted but at an earlier point in time, i.e., before the agent is brought in contact with the cell.

The methods of the invention may also comprise comparing the cellular parameter in the first cell after contacting the first cell with the agent to the cellular parameter in the control cell. A difference in the cellular parameter in the first cell after contacting the first cell with the agent relative to the cellular parameter in the control cell will indicate whether the agent that interacts with T-cadherin mimics an action of adiponectin.

For instance, an agent will be identified as one that mimics an action of adiponectin if: (i) the cellular parameter that is measured is one that is known to increase upon contacting cells with adiponectin, and (ii) the cellular parameter in the first cell after contacting the first cell with the agent is greater than the cellular parameter in the control cell. For example, if the cellular parameter that is measured is fatty acid oxidation, glucose uptake, lactate production, AMPK phosphorylation or ACC phosphorylation, then an agent will be identified as one that mimics an action of adiponectin if the cellular parameter in the first cell after contacting the first cell with the agent is greater than the cellular parameter in the control cell.

An agent will also be identified as one that mimics an action of adiponectin if: (i) the cellular parameter that is measured is one that is known to decrease upon contacting cells with adiponectin, and (ii) the cellular parameter in the first cell after contacting the first cell with the agent is less than the cellular parameter in the control cell. For example, if the cellular parameter that is measured is insulin-dependent glucose output, then an agent will be identified as one that mimics an action of adiponectin if the level of insulin-dependent glucose output in the first cell after contacting the first cell with the agent is less than the level of insulin-dependent glucose output in the control cell.

The first and second cells can be any cells that are capable of exhibiting a cellular parameter that is the same or similar to a cellular parameter that is influenced by the interaction of a cell with adiponectin. The first and second cells may naturally exhibit a cellular parameter that is the same or similar to a cellular parameter that is influenced by the interaction of a cell with adiponectin. Alternatively, the first and second cells may be engineered to exhibit a cellular parameter that is the same or similar to a cellular parameter that is influenced by the interaction of a cell with adiponectin. Exemplary cells that naturally exhibit a change in cellular parameter in response to their interaction with adiponectin are liver cells, muscle cells (e.g., smooth muscle cells (e.g., human aortic smooth muscle cells (HASMCs)), skeletal muscle cells, myoblasts, etc), and endothelial cells (e.g., human aortic endothelial cells (HAECs).

The invention also includes methods for determining whether a test agent mimics an action of adiponectin, said methods comprising: (a) contacting a first cell with a test agent, wherein the first cell expresses T-cadherin; (b) contacting a second cell with the test agent, wherein the second cell does not express T-cadherin; (c) measuring a cellular parameter in the first and second cells after contacting the first and second cells with the test agent; and (d) comparing the cellular parameter in the first cell to the cellular parameter in the second cell after contacting the first and second cells with the test agent.

In a preferred embodiment, the first and second cells are substantially identical to one another except for the expression of T-cadherin: the first cell expressing T-cadherin and the second cell not expressing T-cadherin. Preferably, T-cadherin is expressed on the surface of the first cell. Thus, any difference in cellular parameter observed between the first and second cells after contacting them with the test agent will reflect the interaction of the test agent with T-cadherin.

The cellular parameter that is measured is any parameter that is influenced, e.g., increased, enhanced, stimulated, decreased, attenuated, suppressed, etc., by adiponectin. Exemplary cellular parameters include, e.g., fatty acid oxidation, glucose uptake or output, lactate production, 5′-AMP-activated protein kinase (AMPK) phosphorylation, acetyl coenzyme A carboxylase (ACC) phosphorylation, IRS-1-mediated PI-3-kinase activity, smooth muscle cell proliferation and/or migration, NF-κB signaling, cAMP production and TNF-α-induced expression of endothelial adhesion molecules (e.g., vascular cell adhesion molecule-1 (VCAM-1), endothelial-leukocyte adhesion molecule-1 E-selectin), and intracellular adhesion molecule-1 (ICAM-1)). Other cellular parameters include, cellular adhesion (e.g., monocyte adhesion to endothelium), myeloid differentiation, macrophage cytoline production, phagocytosis, lipid accumulation, and uptake of acetylated low-density lipoprotein particles. (See Berg et al., Trends Endocrinol. & Metab. 13:84-89 (2002)). Methods for measuring such cellular parameters are known in the art. (See, e.g., Berg et al., Trends Endocrinol. & Metab. 13:84-89 (2002); Yamauchi et al., Nat. Med. 8:1288-1295 (2002)).

According to the methods of the invention, an increase in the cellular parameter measured in the first cell, as compared to the cellular parameter measured in the second cell, after contacting the first and second cells with the test agent, identifies the test agent as one that mimics an action of adiponectin when the cellular parameter is one that is known to increase when adiponectin is administered. A decrease in the cellular parameter measured in the first cell, as compared to the cellular parameter measured in the second cell, after contacting the first and second cells with the test agent, identifies the test agent as one that mimics an action of adiponectin when the cellular parameter is one that is known to decrease when adiponectin is administered.

For example, if the cellular parameter measured is AMPK phosphorylation, and, after contacting the first and second cells with the test agent the level of AMPK phosphorylation is greater in the first cell (which expresses T-cadherin) than in the second cell (which does not express T-cadherin), then the test agent is identified as one that mimics an action of adiponectin.

The test agent, in certain embodiments, is an antibody. The test agent may, for example, be an antibody that interacts with T-cadherin. The antibody may be a monoclonal antibody.

The cells used in the methods of the invention can be any cells that are capable of exhibiting a cellular parameter that is the same or similar to a cellular parameter that is influenced by the interaction of a cell with adiponectin. The cells used in the practice of the invention may naturally exhibit a cellular parameter that is the same or similar to a cellular parameter that is influenced by the interaction of a cell with adiponectin. Alternatively, the first and/or second cells may be engineered to exhibit a cellular parameter that is the same or similar to a cellular parameter that is influenced by the interaction of a cell with adiponectin.

The invention also includes methods for determining whether a test agent inhibits or enhances the interaction between adiponectin and T-cadherin. According to certain embodiments, the methods of the invention comprise: (a) providing a test mixture comprising: (i) adiponectin or a fragment thereof and (ii) T-cadherin or a fragment thereof, one of them being fused to a detectable marker or enzyme and the other being immobilized on a suitable carrier, and (iii) a test agent; (b) providing a control mixture comprising: (i) adiponectin or a fragment thereof and (ii) T-cadherin or a fragment thereof, one of them being fused to a detectable marker or enzyme and the other being immobilized on a suitable carrier; (c) removing unbound adiponectin and T-cadherin from the test mixture and from the control mixture; and (d) measuring the signal produced by the marker or enzyme in the test mixture and in the control mixture.

According to other embodiments, the methods of the invention comprise: (a) providing a test mixture comprising: (i) adiponectin or a fragment thereof and (ii) T-cadherin or a fragment thereof, one of them being fused to a detectable marker or enzyme and the other being expressed on the surface of a cell, and (iii) a test agent; (b) providing a control mixture comprising: (i) adiponectin or a fragment thereof and (ii) T-cadherin or a fragment thereof, one of them being fused to a detectable marker or enzyme and the other being expressed on the surface of a cell; (c) removing unbound adiponectin and T-cadherin from the test mixture and from the control mixture; and (d) measuring the signal produced by the marker or enzyme in the test mixture and in the control mixture.

Adiponectin, T-cadherin, and fragments and variants thereof, can be obtained from any source or method available to those of ordinary skill in the art. Adiponectin, T-cadherin, and fragments thereof, for example, can be obtained from expression vectors comprising nucleotide sequences which express the molecules and/or fragments in appropriate cells. Nucleic acid molecules that encode adiponectin or T-cadherin from a variety of organisms are known in the art. Such nucleic acid molecules can be cloned directly into expression vectors. Alternatively, the nucleic acid molecules can be manipulated and/or mutated prior to cloning them into expression vectors. Such manipulations include, but are not limited to, altering individual nucleotides or groups of nucleotides, truncations, insertions, deletions, and addition of amino acid sequences that are subject to post-translational modifications.

Fragments and derivatives and variants of Adiponectin and T-cadherin, may contain minor modifications of the sequences of Adiponectin and T-cadherin which do not destroy its immunoreactivity. Limited modification may be made without destroying the biological function of T-cadherin and Adiponectin, and only a portion of the entire primary structure may be required to effect activity. Such minor modifications may result in proteins which have substantially equivalent or enhanced function. Exemplary fragments of Adiponectin may comprise, or alternatively essentially consist of, or alternatively consist of the globular domain or the collagen domain of Adiponectin. Exemplary fragments of T-cadherin may comprise, or alternatively essentially consist of, or alternatively consist of one or more of the five cadherin domains of T-cadherin, preferably the cadherin domain 1 of T-cadherin (SEQ ID NO: 55).

According to certain aspects of the invention, adiponectin, fragments of adiponectin, T-cadherin, or fragments of T-cadherin are fused to a detectable marker or enzyme.

A “detectable marker,” as used herein can include, e.g., any nucleotide sequence that encodes a polypeptide that produces a signal or that can be specifically detected using one or more reagents that interact with the polypeptide or that detect a chemical reaction involving the polypeptide.

Exemplary detectable markers include epitope tags that can be recognized by specific antibodies or binding reagents (e.g., FLAG, Strep, poly-histidine, VSV-G, hemaglutanin, c-myc and the tripeptide Glu-Glu-Phe), and amino acid sequences that are post-translationally modified. Exemplary post-translational modifications that can be used with the present invention include biotinylation, attachment of 4-phosphopanthetheine, attachment of lipoic acid and attachment of flavins and glycosylation. Further details regarding post-translational modifications of amino acid sequences can be found in U.S. Pat. No. 5,252,466 and the references cited therein. The detectable marker can also be a radiolabel (e.g., 14C or 3H), dyes and metal sols.

The detectable marker, in some instances may be a label or a molecular species that physically interacts with a label. A label may be any detectable composition whereby the detection can be spectroscopic, photochemical, biochemical, immunochemical, physical or chemical. For example, useful labels can include 32P, 35S, 3H, 14C, 125I, 131I, fluorescent dyes (e.g. FITC, rhodamine and lanthanide phosphors), electron-dense reagents, enzymes, e.g. as commonly used in ELISA (e.g. horseradish peroxidase, beta-galactosidase, luciferase and alkaline phosphatase), biotin, dioxigenin, or haptens and proteins for which antisera or monoclonal antibodies are available. The label may be directly incorporated into a target molecule (e.g., adiponectin or T-cadherin) to be detected, or it may be attached to a probe or antibody which binds to the target.

The expression “fused to an enzyme” is intended to mean that adiponectin, fragments of adiponectin, T-cadherin, or fragments of T-cadherin are covalently or non-covalently attached to an enzyme that participates in one or more chemical reactions, the products or intermediates of which can be detected with particular reagents. For example, certain enzymes catalyze reactions that produce molecules which cause a color change when combined with other chemical ingredients. Other enzymes produce chemical intermediates that can be detected using various chemical reagents and instruments.

According to certain aspects of the invention, when adiponectin or a fragment of adiponectin is fused to a detectable marker or enzyme, then T-cadherin or a fragment of T-cadherin will be immobilized on a suitable carrier. Conversely, when T-cadherin or a fragment of T-cadherin is fused to a detectable marker or enzyme, then adiponectin or a fragment of adiponectin will be immobilized on a suitable carrier.

The term “suitable carrier,” as used herein is intended to mean any solid surface to which a polypeptide can be attached, either directly or indirectly. Suitable carriers include, e.g., beads, matrices, and other solid surfaces, including, e.g., petri dishes, microtiter wells, test tubes, microscope slides and coverslips, etc. Methods for immobilizing polypeptides to carriers are well known in the art.

According to certain other aspects of the invention, when adiponectin or a fragment of adiponectin is fused to a detectable marker or enzyme, then T-cadherin or a fragment of T-cadherin will be expressed on the surface of a cell. Conversely, when T-cadherin or a fragment of T-cadherin is fused to a detectable marker or enzyme, then adiponectin or a fragment of adiponectin will be expressed on the surface of a cell.

Methods for expressing proteins on the surface of a cell are well known in the art. For example, the protein can be expressed such that it is attached or fused to a sequence that is normally expressed on the surface of a cell or delivered to the surface of a cell. Any type of cell can be used to express T-cadherin or adiponectin on its surface, including, e.g., bacterial cells, yeast cells, mammalian cells, insect cells and other animal cells.

In order to determine whether a test agent inhibits or enhances the interaction between adiponectin and T-cadherin (or fragments thereof), the signal produced by the marker or enzyme in the test mixture, (containing the test agent) is compared to the signal produced by the marker or enzyme in the control mixture (which does not contain the test agent).

Before measuring the signal produced by the marker or enzyme, it is preferred that unbound adiponectin and T-cadherin are first removed from the test mixture and from the control mixture. The expression “unbound adiponectin and T-cadherin” is intended to mean adiponectin, fragments of adiponectin, T-cadherin and fragments of T-cadherin that are not attached, directly or indirectly (e.g., through the interaction with a polypeptide), to the suitable carrier or to the surface of the cell.

For example, when T-cadherin is immobilized on a suitable carrier, and a labeled adiponectin is added, it is desired that any adiponectin that is not attached to the suitable carrier (via its interaction with immobilized T-cadherin) be removed from the test and control mixtures prior to measuring the signal produced from the marker or enzyme. By removing unbound adiponectin and T-cadherin, the signal that is measured will more accurately reflect the extent to which adiponectin and T-cadherin interact.

Likewise, when T-cadherin is expressed on the surface of a cell, and a labeled adiponectin is added, it is desired that any adiponectin that is not attached to the surface of the cell (via its interaction with surface-expressed T-cadherin) be removed from the test and control mixtures prior to measuring the signal produced from the marker or enzyme. By removing unbound adiponectin and T-cadherin, the signal that is measured will more accurately reflect the extent to which adiponectin and T-cadherin interact.

In some embodiments, immunoprecipitation may be used to separate bound and free labeled components. An antibody may be employed to bring an unlabelled component out of solution (whether or not this component has bound to another labeled component or not). After separation, the label present in solution (free) and the label present in or on the solid phase (bound) may be measured. Standard analyses of such bound and free data, e.g. Scatchard plots and the determination of affinity and inhibition constants for binding are well known to the person of ordinary skill in the art.

Where the solid phase is not particulate, e.g. in the form of a surface, such as a microtiter plate well, then binding assays measuring bound and free label may be performed but this will normally involve the removal of liquid phase from the wells after binding reactions have occurred. Advantageously, this assay format may dispense with the need for providing specifically labeled reaction components. Instead, labeled antibodies may be used to measure the binding of previously free reaction components to solid phase components.

Immunological binding assays are known in the art. For a review, see Methods in Cell Biology Vol. 37: Antibodies in Cell Biology, Asai, (Ed.) Academic Press, Inc. New York (1993).

Throughout the assays of the invention, incubation and/or washing steps may be required after each application of reagent or incubation of combinations of reagents. Incubation steps may vary from about 5 minutes to several hours, perhaps from about 30 minutes to about 6 hours. However, the incubation time usually depends upon the assay format, analyte, volume of solution, concentrations, and so forth. Usually, the assays should be carried out at ambient temperature, although they may be conducted at temperatures; in the range 10° C. to 40° C., for example. One possible assay format is an enzyme-linked immunosorbent assay (ELISA).

After unbound adiponectin and T-cadherin are removed from the test mixture and from the control mixture, the signal produced by the marker or enzyme is measured in the test mixture and in the control mixture. The signal can be measured using any technique that is appropriate in view of the particular detectable signal. For example, the signal can be measured using a device that measures color or ultraviolet absorption, turbidity, etc. Alternatively, the measurement can be made manually, e.g., by eye, as long as a consistent measurement scale is employed.

A decrease in the signal produced by the marker or enzyme in the test mixture as compared to the signal produced by the marker or enzyme in the control mixture indicates the ability of the test agent to inhibit the interaction between adiponectin and T-cadherin.

By contrast, an increase in the signal produced by the marker or enzyme in the test mixture as compared to the signal produced by the marker or enzyme in the control mixture indicates the ability of the test agent to enhance the interaction between adiponectin and T-cadherin.

The test agent can be any molecule or chemical compound. The test agent may be, e.g., an antibody. In certain embodiments, the test agent is an antibody that interacts with T-cadherin. The antibody that interacts with T-cadherin may be a monoclonal antibody.

The invention also includes methods for identifying polypeptides that interact with adiponectin. The methods, according to this aspect of the invention, comprise: (a) obtaining a population of cells comprising two or more cells that express different candidate polypeptides on their surface (the candidate polypeptides are those that are tested for their interaction with adiponectin); (b) contacting the population of cells with a bait polypeptide; (c) separating cells which have the bait polypeptide bound to them from cells that do not have the bait polypeptide bound to them; and (d) identifying the candidate polypeptide that is expressed on the surface of the cells which have the bait polypeptide bound to them. The candidate polypeptide that is expressed on the surface of the cells that have the bait polypeptide bound to them is a polypeptide that interacts with adiponectin.

According to this aspect of the invention, the “bait polypeptide” is adiponectin, a fragment of adiponectin, adiponectin fused to a detectable marker or enzyme, a fragment of adiponectin fused to a detectable marker or enzyme, or any other variant of adiponectin.

Cells which have the bait polypeptide bound to them can be separated from cells that do not have the bait polypeptide bound to them using methods that are well known in the art; e.g., bringing the cells in contact with a composition comprising a solid surface or matrix to which an agent that interacts with the bait polypeptide is attached. The solid surface or matrix can then be removed from any unbound cells, bringing with it the cells which have the bait polypeptide bound to them and separating such cells from the cells that do not have the bait polypeptide bound to them.

When the bait polypeptide is adiponectin fused to a detectable marker, or a fragment of adiponectin fused to a detectable marker, and the detectable marker emits a fluorescent signal or can interact, directly or indirectly, with an agent that emits a fluorescent signal (e.g., a fluorescently labeled antibody), then the separating step can be accomplished using fluorescence activated cell sorting (FACS). The process of FACS is well known in the art.

The candidate polypeptides that are expressed on the surface of the cells in the population of cells can, in certain embodiments, be expressed from expression vectors within the cells. For example, an expression library that expresses a variety of candidate polypeptides on the surface of cells can be introduced into an appropriate cell type, and the resulting population of cells (each expressing a different member of the library) can be used in the methods of the invention.

After cells which have the bait polypeptide bound to them are separated from the cells that do not have the bait polypeptide bound to them, the candidate polypeptide that is expressed on the surface of the cells that have the bait polypeptide bound to them can be identified. For example, if the candidate polypeptide is expressed from an expression vector, the vector can be isolated from the cells, and the nucleic acid sequence of the candidate polypeptide can be determined using routine methods in the art. The amino acid sequence can be translated from the nucleic acid sequence, and the identity of the polypeptide that is expressed on the surface of the cells can be ascertained. Alternatively, the amino acid sequence of the polypeptide that is expressed on the surface of the cells to which the bait polypeptide binds can be ascertained using methods that are well known in the art.

The methods of the invention, or certain aspects thereof, may be carried out in the context of high-throughput screening assays. For example, automated systems and devices can be used to process samples, to combine reagents, to remove unbound proteins, to measure and record various cellular/physiological parameters and to perform other steps and procedures involved in the methods. High-throughput systems may be employed to assay hundreds or thousands of samples at the same time or in succession. Adapting any of the methods of the invention to high-throughput screening formats can be accomplished by those of ordinary skill in the art.

In another aspect of the invention, a method is provided for the production of a pharmaceutical composition. The method according to this aspect of the invention comprises identifying an agent capable of mimicking an action of adiponectin or capable of modulating the adiponectin-T-cadherin interaction and furthermore mixing the compound, or a derivative or homologue thereof, with a pharmaceutically acceptable carrier (or excipient). Identifying such an agent can be carried out by any of the methods of the invention described elsewhere herein. The agent, in some embodiments, may be an antibody, e.g., a monoclonal antibody.

Suitable carriers or excipients are well-known in the art. A carrier or excipient may be a solid, semi-solid or liquid material which may serve as a vehicle or medium for the active ingredient. One of ordinary skill in the art can readily select the proper form and mode of administration depending upon the particular characteristics of the product selected, the disease or condition to be treated, the stage of the disease or condition, and other relevant circumstances (Remington's Pharmaceutical Sciences, Mack Publishing Co. (1990)). The proportion and nature of the pharmaceutically acceptable carrier or excipient are determined by the solubility and chemical properties of the pharmaceutically active compound selected, the chosen route of administration, and standard pharmaceutical practice. The pharmaceutical preparation may be adapted for oral, parenteral or topical use and may be administered to the patient in the form of tablets, capsules, suppositories, solution, suspensions, or the like. The pharmaceutically active agents of the present invention, while effective themselves, can be formulated and administered in the form of their pharmaceutically acceptable salts, such as acid addition salts or base addition salts, for purposes of stability, convenience of crystallization, increased solubility, and the like.

In a further embodiment, an agent identified by a method of the invention is provided for the treatment or the prophylactic treatment of a disease or condition including, preferably selected from, hypoadiponectinemia, obesity, anorexia nervosa, coronary artery disease (CAD), type I diabetes, type II diabetes. The agent, in some embodiments, may be an antibody, e.g., a monoclonal antibody.

Furthermore, a method of treating and/or preventing a disease or condition in a mammal is provided, wherein said disease or condition is selected from a disease or condition including, preferably selected from the group consisting of: hypoadiponectinemia, obesity, anorexia nervosa, coronary artery disease, type I diabetes, type II diabetes, and atherosclerosis. The methods according to this aspect of the invention comprises administering to the mammal a therapeutically effective amount of an agent identified by a method of the invention. The compound, in some embodiments, may be an antibody, e.g., a monoclonal antibody.

In another embodiment, the use of T-cadherin is provided for the preparation of a medicament capable of mimicking an action of adiponectin. In an alternative embodiment, T-cadherin may be used for the preparation of a medicament for treating and/or preventing diseases or conditions including, preferably selected from, hypoadiponectinemia, obesity, anorexia nervosa, coronary artery disease, type I diabetes, type II diabetes, atherosclerosis.

In a further aspect, an agent identified by a method of the invention that specifically binds T-cadherin or a fragment thereof may be provided for use as a medicament for treating and/or preventing diseases or conditions including, preferably selected from, hypoadiponectinemia, obesity, anorexia nervosa, coronary artery disease, type I diabetes, type II diabetes, atherosclerosis. The compound, in some embodiments, may be an antibody, e.g., a monoclonal antibody.

In another aspect of the invention, an agent identified by a method of the invention that specifically binds T-cadherin or a fragment thereof, may be used for the preparation of a medicament capable of mimicking an action of adiponectin. Alternatively, the compound identified by a method of the invention that specifically binds T-cadherin may be used for the preparation of a medicament for treating and/or preventing diseases or conditions including hypoadiponectinemia, obesity, anorexia nervosa, coronary artery disease, type I diabetes, type II diabetes, and atherosclerosis. The agent, in some embodiments, may be an antibody, e.g., a monoclonal antibody.

Fragments and derivatives and variants of T-cadherin may comprise, or alternatively essentially consist of, or alternatively consist of one or more of the five cadherin domains of T-cadherin (SEQ ID NO: 55, SEQ ID NO: 56, SEQ ID NO: 57, SEQ ID NO: 58, SEQ ID NO: 59), preferably of the cadherin domain 1 of T-cadherin (SEQ ID NO: 55).

Also provided by the invention are agents identified by a method of the invention formulated into a pharmaceutical composition, possibly in the presence of suitable excipients known to one of ordinary skill in the art. The compositions may be administered in the form of any suitable composition by any suitable method of administration within the knowledge of a person of ordinary skill in the art.

An exemplary route of administration is parenterally. In parenteral administration, the compositions of this invention will be formulated in a unit dosage injectable form such as a solution, suspension or emulsion, in association with a pharmaceutically acceptable excipient. Such excipients are inherently nontoxic and nontherapeutic. Examples of such excipients are saline, Ringer's solution, dextrose solution and Hank's solution. Nonaqueous excipients such as fixed oils and ethyl oleate may also be used. A preferred excipient is 5% dextrose in saline. The excipient may contain minor amounts of additives such as substances that enhance isotonicity and chemical stability, including buffers and preservatives.

The agent, if it is a protein, is preferably administered at a concentration that is therapeutically effective to prevent allograft rejection, GVHD, allergy and autoimmune diseases. The dosage and mode of administration will depend on the individual. Generally, the compositions are administered so that the functional protein is given at a dose between 1 pg/kg and 10 mg/kg, more preferably between 10 ug/kg and 5 mg/kg, most preferably between 0.1 and 2 mg/kg. Preferably, it is given as a bolus dose. Continuous short time infusion (during 30 minutes) may also be used. The compositions according to the invention may be infused at a dose between 5 and 20 μg/kg/minute, more preferably between 7 and 15 μg/kg/minute.

In certain instances, the “therapeutically effective amount” of an agent needed is determined as being the amount sufficient to cure the patient in need of treatment or at least to partially arrest the disease and its complications. Amounts effective for such use will depend on the severity of the disease and the general state of the patient's health. Single or multiple administrations may be required depending on the dosage and frequency as required and tolerated by the patient.

The invention also provides isolated receptor-ligand complexes comprising: (a) adiponectin or a fragment or variant thereof; and (b) T-cadherin or a fragment of variant thereof; wherein said adiponectin or a fragment or variant thereof is in contact with said T-cadherin or a fragment of variant thereof. For example, the receptor-ligand complexes of the invention may comprise adiponectin and T-cadherin, wherein adiponectin and T-cadherin are covalently or non-covalently attached to each other.

The invention also provides isolated receptor-ligand complexes comprising: (a) adiponectin or a fragment or variant thereof; and (b) an adiponectin receptor; wherein said adiponectin or a fragment or variant thereof is in contact with said adiponectin receptor.

As used herein, the expression “in contact with” is intended to encompass circumstances wherein the attachment between adiponectin and T-cadherin or another adiponectin receptor is direct (e.g., wherein adiponectin and T-cadherin are in direct contact with one another), and circumstances wherein the attachment is indirect (e.g., wherein adiponectin and T-cadherin are not in direct contact with one another but are each in contact with one or more common molecules).

The receptor-ligand complexes of the invention are useful for, e.g., generating antibodies that recognize adiponectin/T-cadherin complexes, immunizing animals, treating individuals afflicted with diseases and conditions such as obesity, anorexia nervosa, type I or type II diabetes, and coronary artery disease, and diagnosing diseases and conditions such as obesity, anorexia nervosa, type II diabetes, and coronary artery disease. Other uses for the receptor-ligand complexes of the invention will be appreciated by those of ordinary skill in the art.

In a preferred aspect, said adiponectin is encoded by a nucleotide sequence selected from the group consisting of: (i) a nucleotide sequence comprising the coding region (CDS) of a sequence as set forth in SEQ ID NO: 6; (ii) a nucleotide sequence with at least 80% identity, to any of the sequences of (i); (iii) a nucleic acid that hybridizes to a nucleotide sequence of (i), or (ii); (iv) a nucleotide sequence complementary to any of the nucleotide sequences in (i), (ii), or (iii); or (v) a fragment or variant of any of the nucleotide sequences of (i), (ii), (iii), or (iv) that hybridizes to a nucleotide sequence of (i).

In an other aspect of the invention, a receptor-ligand complex is provided comprising adiponectin and its receptor, wherein said adiponectin has an amino acid sequence selected from the group consisting of: (i) an amino acid sequence as set forth in any one of the sequences selected from SEQ ID NO: 4 to SEQ ID NO:5, SEQ ID NO:7 to SEQ ID NO:10, and SEQ ID NO: 20; (ii) an amino acid sequence with at least 80% identity to any of the sequences of (i); or (iii) a fragment or variant of any of the amino acid sequences of (i), or (ii).

In one aspect of the invention, the receptor-ligand complex comprises an adiponectin which exhibits at least 80% identity to the sequence of (i), or which comprises a variant of the amino acid sequence shown in any one of the sequences selected from SEQ ID NO: 4 to SEQ ID NO:5, SEQ ID NO:7 to SEQ ID NO:10, and SEQ ID NO: 20, such as a variant comprising an amino acid deletion, addition (e.g. fusion proteins) or substitution of the amino acid sequence shown in any one of the sequences selected from SEQ ID NO: 4 to SEQ ID NO:5, SEQ ID NO:7 to SEQ ID NO:10, and SEQ ID NO: 20. Preferably, the variant comprises a conservative substitution of at least one amino acid in said amino acid sequence in any one of the sequences selected from SEQ ID NO: 4 to SEQ ID NO:5, SEQ ID NO:7 to SEQ ID NO:10, and SEQ ID NO: 20. In a preferred embodiment, said adiponectin has an amino acid sequence shown in any one of the sequences selected from SEQ ID NO: 4 to SEQ ID NO:5, SEQ ID NO:7 to SEQ ID NO:10, and SEQ ID NO: 20.

Preferably, the amino acid sequence of adiponectin comprises a conservative substitution of at least one amino acid of the amino acid sequence of the sequences selected from SEQ ID NO: 4 to SEQ ID NO:5, SEQ ID NO:7 to SEQ ID NO:10, and SEQ ID NO: 20. Useful fragments may exhibit an epitope recognized by polyclonal antibodies raised against the polypeptide having the amino acid sequence shown in any of the sequences selected from SEQ ID NO: 4 to SEQ ID NO:5, SEQ ID NO:7 to SEQ ID NO:10, and SEQ ID NO: 20 for example. A particularly preferred polypeptide is that encoded by any one of the amino acid sequence shown in the sequences selected from SEQ ID NO: 4 to SEQ ID NO:5, SEQ ID NO:7 to SEQ ID NO:10, and SEQ ID NO: 20. Also provided are antibodies that are specifically reactive against the polypeptides of the receptor-ligand complex of the invention.

The present invention further provides a receptor-ligand complex comprising adiponectin and its receptor, wherein said adiponectin receptor is encoded by a nucleotide sequence selected from the group consisting of: (i) a nucleotide sequence as set forth in any one of the sequences selected from SEQ ID NO:21, SEQ ID NO:23, SEQ ID NO:25, SEQ ID NO:27, SEQ ID NO:29, SEQ ID NO:31, SEQ ID NO:33 to SEQ ID NO:47, SEQ ID NO:49, SEQ ID NO:51, and SEQ ID NO:53; (ii) a nucleotide sequence with at least 80% identity to any of the sequences of (i); (iii) a nucleic acid that hybridizes to a nucleotide sequence of (i), or (ii); (iv) a nucleotide sequence complementary to any of the nucleotide sequences in (i), (ii), or (iii); and (v) a fragment of any of the nucleotide sequences of (i), (ii), (iii), or (iv) that hybridizes to a nucleotide sequence of (i).

In another embodiment, the receptor-ligand complex of the invention comprises an adiponectin receptor, wherein said adiponectin receptor has an amino acid sequence selected from the group consisting of: (i) an amino acid sequence as set forth in any one of the sequences selected from SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54, SEQ ID NO: 55, SEQ ID NO: 56, SEQ ID NO: 57, SEQ ID NO: 58, and SEQ ID NO: 59; (ii) an amino acid sequence with at least 80% identity to any of the sequences of (i); or (iii) a fragment or variant of any of the amino acid sequences of (i), or (ii).

In one aspect of the invention, the isolated and purified receptor-ligand complex comprises an adiponectin receptor which exhibits at least 80% identity to the sequence of (i), or which comprises a variant of the amino acid sequence shown in any one of the sequences selected from SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54, SEQ ID NO: 55, SEQ ID NO: 56, SEQ ID NO: 57, SEQ ID NO: 58, and SEQ ID NO: 59, such as a variant comprising an amino acid deletion, addition (e.g. fusion proteins) or substitution of the amino acid sequence shown in any one of the sequences selected from SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54, SEQ ID NO: 55, SEQ ID NO: 56, SEQ ID NO: 57, SEQ ID NO: 58, and SEQ ID NO: 59. Preferably, the variant comprises a conservative substitution of at least one amino acid in said amino acid sequence in any one of the sequences selected from SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54, SEQ ID NO: 55, SEQ ID NO: 56, SEQ ID NO: 57, SEQ ID NO: 58, and SEQ ID NO: 59. In a preferred embodiment, said adiponectin receptor has an amino acid sequence shown in any one of the sequences selected from SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54, SEQ ID NO: 55, SEQ ID NO: 56, SEQ ID NO: 57, SEQ ID NO: 58, and SEQ ID NO: 59.

Preferably, the amino acid sequence of said adiponectin receptor comprises a conservative substitution of at least one amino acid of the amino acid sequence of the sequences selected from SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54, SEQ ID NO: 55, SEQ ID NO: 56, SEQ D NO: 57, SEQ ID NO: 58, SEQ ID NO: 59. Useful fragments may exhibit an epitope recognized by polyclonal antibodies raised against the polypeptide having the amino acid sequence shown in any of the sequences selected from SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54, SEQ ID NO: 55, SEQ ID NO: 56, SEQ ID NO: 57, SEQ ID NO: 58, and SEQ ID NO: 59 for example. A particularly preferred polypeptide is that which comprises any one of the amino acid sequence shown in the sequences selected from SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54 SEQ ID NO: 55, SEQ ID NO: 56, SEQ ID NO: 57, SEQ ID NO: 58, and SEQ ID NO: 59, preferably SEQ ID NO: 55. Also provided are antibodies that are specifically reactive against the polypeptides of the receptor-ligand complex of the invention.

Also included within the invention are variants and derivatives of the polypeptide described by SEQ ID NO: 4 to SEQ ID NO:5, SEQ ID NO:7 to SEQ ID NO:10, and SEQ ID NO: 20 or fragment thereof or by SEQ ID NO:22, SEQ ID NO:24, SEQ ID NO:26, SEQ ID NO:28, SEQ ID NO:30, SEQ ID NO:32, SEQ ID NO:48, SEQ ID NO:50, SEQ ID NO:52, SEQ ID NO:54, SEQ ID NO: 55, SEQ ID NO: 56, SEQ ID NO: 57, SEQ ID NO: 58, or SEQ ID NO: 59 or fragments thereof, whether produced by recombinant means or synthetic means or isolated from naturally occurring sources. For example, peptides having modified amino acids/peptide linkages, and peptides containing non-naturally occurring amino acids and/or cyclic peptides, which may have improved properties such as stability or activity are included. In addition the peptides of the invention may be in the form or a fusion with another protein, for example, tags for the targeted delivery or detection of the polypeptide (including fragments thereof).

Variants of the polypeptides included within the receptor-ligand complexes of the invention include all forms of mutant variants, for example wherein at least one amino acid is deleted or substituted. Any changes involving substitution of amino acids are preferably neutral or conservative substitutions. Other variants include proteins or polypeptides comprising at least one additional amino acid in the sequence, and/or further comprising an additional amino acid sequence or domain, such as fusion proteins, as is well known in the art.

Further variants of the polypeptides included within the receptor-ligand complexes of the invention include those wherein at least one of the amino acids in the sequence is a natural or unnatural analogue. Also, one or more amino acids in the sequence may be chemically modified, e.g. to increase physical stability or to lower susceptibility to enzymatic, particularly protease or kinase, activity. Fragments and derivatives and variants of Adiponectin and T-cadherin, may contain minor modifications of the sequences of Adiponectin and T-cadherin which do not destroy its immunoreactivity. Limited modification may be made without destroying the biological function of T-cadherin and Adiponectin, and only a portion of the entire primary structure may be required to effect activity. Such minor modifications may result in proteins which have substantially equivalent or enhanced function. Exemplary fragments of Adiponectin may comprise, or alternatively essentially consist of, or alternatively consist of the globular domain or the collagen domain of Adiponectin. Exemplary fragments of T-cadherin may comprise, or alternatively essentially consist of, or alternatively consist of one or more of the five cadherin domains of T-cadherin (SEQ ID NO: 55, SEQ ID NO: 56, SEQ ID NO: 57, SEQ ID NO: 58, SEQ ID NO: 59), preferably the cadherin domain 1 of T-cadherin (SEQ ID NO: 55).

The invention also provides antibodies that are specifically reactive against the receptor-ligand complex (e.g., a T-cadherin-adiponectin complex) of the invention or against T-cadherin. Methods for producing antibodies are well known in the art. An antibody specific for the polypeptide of the invention can be easily obtained by immunizing an animal with an immunogenic amount of the polypeptide. Therefore, an antibody recognizing a particular polypeptide embraces both polyclonal antibodies and antisera which are obtained by immunizing an animal, and which can be confirmed to recognize the polypeptide of this invention by Western blotting, ELISA, immunostaining or other routine procedure known in the art.

It is well known that if a polyclonal antibody can be obtained by sensitization, a monoclonal antibody is secreted by the hybridoma, which may be obtained from the lymphocytes of the sensitized animal (Chapter 6, Antibodies A Laboratory Manual, Cold Spring Harbor Laboratory Press, 1988). Therefore, monoclonal antibodies recognizing the polypeptide of the invention are also provided. Methods of producing polyclonal and monoclonal antibodies are known to those of skill in the art and described in the scientific and patent literature, see, e.g., Coligan, Current Protocols in Immunology, Wiley/Green, NY (1991); Stites (eds.) Basic and Clinical Immunology (7th ed.) Lange Medical Publications, Los Altos, Calif., and references cited therein (Stites); Goding, Monoclonal Antibodies: Principles and Practice (2nd ed.) Academic Press, New York, N.Y. (1986); and Kohler (1975) Nature 256: 495. Such techniques include selection of antibodies from libraries of recombinant antibodies displayed in phage or similar on cells. See, Huse (1989) Science 246: 1275 and Ward (1989) Nature 341: 544. Recombinant antibodies can be expressed by transient or stable expression vectors in mammalian cells, as in Norderhaug (1997) J. Immunol. Methods 204: 77-87.

According to the invention, an “antibody” also embraces an active fragment thereof. An active fragment means a fragment of an antibody having activity of antigen-antibody reaction. Specifically named, these are active fragments, such as F(ab′)2, Fab′, Fab, and Fv. For example, F(ab′)2 results if the antibody of this invention is digested with pepsin, and Fab results if digested with papain. Fab′ results if F(ab′)2 is reduced with a reagent such as 2-mercaptoethanol and alkylated with monoiodoacetic acid. Fv is a mono active fragment where the variable region of heavy chain and the variable region of light chain are connected with a linker. A chimeric antibody is obtained by conserving these active fragments and substituting the fragments of another animal for the fragments other than these active fragments. In particular, humanized antibodies are envisioned.

The invention further provides a use of a receptor-ligand complex of the invention for the preparation of a medicament for treating and/or preventing diseases or conditions including, preferably selected from, hypoadiponectinemia, obesity, anorexia nervosa, coronary artery disease, type I diabetes, type II diabetes, and atherosclerosis.

Also provided are nucleic acid vectors which encode the receptor-ligand complexes of the invention, as well as host cells comprising the vectors or nucleic acids, and transgenic, knockout or genetically modified animals (other than humans, in particular mice), comprising manipulated nucleic acids of the invention or lacking the endogenous sequence.

In a further aspect, a kit comprising a receptor-ligand complex of the invention is provided.

The invention further provides a method of diagnosing or prognosing diseases or conditions such as, e.g., hypoadiponectinemia, obesity, anorexia nervosa, coronary artery disease, type I diabetes, type II diabetes, and atherosclerosis. Such methods comprise: (i) obtaining a sample from an individual; (ii) analyzing said sample for the presence of a receptor-ligand complex of the invention (e.g., a T-cadherin-adiponectin complex); and (iii) comparing the levels of receptor-ligand complex in the test sample to the level of said complex in healthy tissue; wherein as decrease in receptor-ligand complex concentration in the test sample compared to that of healthy tissue indicates that the individual is at risk for a disease such as hypoadiponectinemia, obesity, anorexia nervosa, coronary artery disease, type I diabetes, type II diabetes, and atherosclerosis.

In a further aspect, the invention provides a method of diagnosing or prognosing diseases or conditions such as, e.g., hypoadiponectinemia, obesity, anorexia nervosa, coronary artery disease, type I diabetes, type II diabetes, and atherosclerosis. Such methods comprise: (i) obtaining a sample from an individual; (ii) analyzing said sample for the presence of T-cadherin or a fragment or variant thereof; and (iii) comparing the levels of T-cadherin in the test sample to the level of T-cadherin in healthy tissue; wherein as decrease in T-cadherin concentration in the test sample compared to that of healthy tissue indicates that the individual is at risk for a disease such as hypoadiponectinemia, obesity, anorexia nervosa, coronary artery disease, type I diabetes, type II diabetes, and atherosclerosis.

An unfavorable prognosis or diagnosis in the meaning of the present invention may be that the individual suffers from a disease or condition including, e.g., a disease or condition selected from, hypoadiponectinemia, obesity, anorexia nervosa, coronary artery disease, type I diabetes, type II diabetes, or atherosclerosis if a decrease of the receptor-ligand complex (e.g., a receptor-ligand complex (e.g., a T-cadherin-adiponectin complex)) of the invention or a decrease of T-cadherin in a sample of an individual compared to a sample of a healthy individual is detected. In one embodiment of the invention, the sample obtained from an individual may be blood serum. In another embodiment of the invention, said sample may be tissue or cells. In a further embodiment of the invention, a method of diagnosing or prognosing diseases or conditions including, e.g., a disease or condition selected from, hypoadiponectinemia, obesity, anorexia nervosa, coronary artery disease, type I diabetes, type II diabetes, and atherosclerosis may be provided, further comprising propagating cells in said sample in cell culture.

The methods of the present invention will typically involve the determination of the presence, level, or activity of the receptor-ligand complex (e.g., a T-cadherin-adiponectin complex) of the invention or of T-cadherin or a fragment thereof in a cell or tissue sample, which sample will often be obtained from a human. The samples tested by the present method can also be obtained from agriculturally important mammals, such as cattle, horses, sheep, etc., or other animals of veterinary interest, such as cats and dogs. The assay may be carried out on any cell or tissue sample, such as somatic tissues, germline tissues, or cancerous tissues, as well as on samples from body fluids, such as pleural fluid, blood, serum, plasma and urine.

A “sample,” according to the invention includes the material being analyzed which is usually, but not necessarily, subjected to pretreatment to provide the receptor-ligand complex (e.g., a T-cadherin-adiponectin complex) of the invention, or the T-cadherin or a fragment thereof, in assayable form. This would normally entail forming a cell extract, methods for which are known in the art (for example, see Scopes, Protein Purification: Principles and Practice, Second Edition (Springer-Verlag, N.Y., 1987)).

In the broader aspects of the invention, there is no limitation on the collection and handling of samples as long as consistency is maintained. The sample is obtained by methods known in the art, such as, biopsies, surgical resections, smears, or the like. Optionally, cells obtained in a sample may be propagated in cell culture.

Consistency of measurement of the receptor-ligand complex (e.g., a T-cadherin-adiponectin complex) of the invention, or of T-cadherin or a fragment thereof, or the activities thereof in clinical samples can be ensured by using a variety of techniques. For example, to control for the quality of each tissue extract, another enzymatic activity, such as alkaline phosphatase, can serve as an internal control. In addition, an internal standard can be measured concurrently with the receptor-ligand complex (e.g., a T-cadherin-adiponectin complex) of the invention or T-cadherin or a fragment thereof in the sample as a control for assay conditions. Thus, the analyzing step can comprise detecting a control protein in the sample, optionally normalizing the value obtained for the receptor-ligand complex (e.g., a T-cadherin-adiponectin complex) of the invention or for T-cadherin or a fragment thereof with a signal obtained with the control protein.

The presence of the receptor-ligand complex (e.g., a T-cadherin-adiponectin complex) of the invention, or of T-cadherin or a fragment thereof, in the sample can be determined by detecting the receptor and/or the ligand using methods known in the art. In this invention, there are no limitations on the type of assay used to measure the receptor-ligand complex (e.g., a T-cadherin-adiponectin complex) of the invention, or the T-cadherin or a fragment thereof, or to measure the activities thereof. For example, the receptor-ligand complex (e.g., a T-cadherin-adiponectin complex) of the invention or of T-cadherin or a fragment thereof can be detected by immunoassays using antibodies specific for the ligand and/or the receptor. The specific antibody may be, e.g., a monoclonal antibody identified by a method of the invention. The antibody can be used, for example, in Western blots of two dimensional gels where the protein is identified by enzyme linked immunoassay or in dot blot (Antibody Sandwich) assays of total cellular protein, or partially purified protein.

Methods for sample concentration and protein purification are described in the literature (see Scopes, 1987). For example, if desired, the receptor-ligand complex (e.g., a T-cadherin-adiponectin complex) of the invention or T-cadherin or a fragment thereof present in the cell extract can be concentrated, by precipitating with ammonium sulfate or by passing the extract through a commercially available protein concentration filter, e.g., an Amicon or Millipore, ultrafiltration unit. The extract can be applied to a suitable purification matrix, such as an anion or a cation exchange resin, or a gel filtration matrix, or subjected to preparative gel electrophoresis. In such cases, the receptor-ligand complex (e.g., a T-cadherin-adiponectin complex) of the invention or the T-cadherin or a fragment thereof and protein yield after each purification step needs to be considered in determining the amount of the receptor-ligand complex (e.g., a T-cadherin-adiponectin complex) of the invention or of T-cadherin or a fragment thereof in a sample.

The receptor-ligand complex (e.g., a T-cadherin-adiponectin complex) of the invention, or T-cadherin or a fragment thereof, may be detected using an antibody specific for the receptor and/or ligand. A control assay can be carried out using, e.g., an antibody specific for another cadherin molecule. Optionally, the method may further comprise correlating in a decrease in the receptor-ligand complex (e.g., a T-cadherin-adiponectin complex) of the invention or of T-cadherin or a fragment thereof in the sample relative to healthy tissue.

The “sample” is preferably a tissue sample mounted onto a solid surface for histochemical analysis. The decrease of detectable, accessible receptor-ligand complex (e.g., a T-cadherin-adiponectin complex) or of T-cadherin or a fragment thereof compared to the amount of detectable, accessible receptor-ligand complex (e.g., a T-cadherin-adiponectin complex) or of T-cadherin or a fragment thereof present in healthy tissue leads to a unfavorable diagnosis or prognosis. If, on the other hand, the amount of detectable, accessible receptor-ligand complex (e.g., a T-cadherin-adiponectin complex) or of T-cadherin or a fragment thereof is the same or increased compared to the amount of detectable, accessible receptor-ligand complex (e.g., a T-cadherin-adiponectin complex) or of T-cadherin or a fragment thereof present in healthy tissue, this leads to a favorable diagnosis or prognosis.

In a preferred embodiment, the invention provides a kit suitable for use in the diagnostic or prognostic methods of the invention. Such kits comprise reagents useful for carrying out these methods, for example, antibodies from one or more species specific for the receptor-ligand complex (e.g., a T-cadherin-adiponectin complex) or for T-cadherin. Secondary antibodies that recognize either or both such primary anti-T-cadherin antibodies can also be included for the purpose of recognition and detection of primary antibody binding to a sample. Such secondary antibodies can be labeled for detection e.g. with fluorophores, enzymes, radioactive labels or otherwise. Other detection labels will occur to those skilled in the art. Alternatively, the primary anti-T-cadherin antibodies can be labeled for direction detection.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

Construction of Adiponectin Expression Vectors

Cloning of Acrp30 and gAcrp30 (=Acrp16)

In order to be able to generate C- and N-terminal fusions of different Adiponectin domains we decided to clone the coding region of the Adiponectin without the endogenous signal peptide. Briefly total RNA was isolated from 60 mg mouse adipose tissue using a Qiagen RNeasy kit according to the manufacturer's recommendation. This total RNA was used for the reverse transcription with an oligo dT primer using the ThermoScript™ RT-PCR System (Life Technologies) according to the manufacturer's recommendation. This RT reaction was then used for the PCR amplification of mouse Adiponectin amino acid (aa) positions 18-247 (=Acrp30 without signal peptide) using the primer pair 30 Fwd: GCGGATCCAGAAGATGACGTTACTACAACTG (SEQ ID NO: 1) Acrp Rev: GCCTCTAGAGAGTTGGTATCATGGTAGAGAAG (SEQ ID NO: 2) and the globular domain aa 104-247 (Acrp16) using the primer pair 16 Fwd: GCGGATCCAAAAGGAGAGCCTGGAGAAGCC (SEQ ID NO: 3) Acrp Rev: GCCTCTAGAGAGTTGGTATCATGGTAGAGAAG (SEQ ID NO: 2).

Both forward primers contain a Bam HI site and the reverse primer an Xba I site allowing the subcloning of Acrp30 (SEQ ID NO: 4) and 16 (SEQ ID NO: 5) respectively. The PCR fragments were subcloned and sequenced. This sequence analysis revealed that aa 113 was changed from Met to Val (ATG→GTG) compared to the published sequence. Since this mutation was found in all PCR amplifications from two independent fat RNA preparations we consider that the published sequence is most likely wrong. Indeed analysis of more than 30 expression sequence tags (EST) revealed that position 113 is not a Met but a Val. The amino acid numbering refers to the published sequence (GenBank, U37222, SEQ ID NO: 7).

Generation of Tagged Adiponectin Versions

The cDNA's encoding Acrp30 and the globular domain of Acrp30 (=Acrp16) were then subcloned into vectors containing either C- or N-terminal FLAG tags. For production purposes these different tagged versions were sub-cloned into pCep-puro (Wuttke et al. J. Biol. Chem. 2001, Sep. 28; 276(39):36839-48). This vector is a modified version of pCep4 (Invitrogen) whereby the hygromycin resistance has been exchanged by the puromycin selection marker. Due to the Epstein-Barr virus origin of replication (OriP) and the replication initiation factor (EBNA1) this plasmid is maintained episomaly at low copy number in 293-EBNA1 cells, allowing the rapid generation of stable cell populations. For details on the sequences of the different proteins see SEQ ID NO: 6 and SEQ ID NO: 7 for Adiponectin (Acrp30), SEQ ID NO: 8 for Acrp30-FLAG-C, SEQ ID NO: 9 for Acrp16-FLAG-C, SEQ ID NO: 10 for Acrp16-FLAG-N.

Eukaryotic Expression of the Baits

In order to produce the different bait proteins 293 EBNA1 cells were transfected with the different Adiponectin constructs. The cells were propagated in DMEM supplemented with 10% FCS, 1% pen/strep and 250 μg/ml G418. One day before transfection the cells were split 1 to 3. The transfections were performed with Lippofectamine 2000 (Invitrogen) according to the manufacturer's recommendation. One day after transfection the cells were transferred from a 10 cm dish to a 15 cm dish in the presence of 1 μg/ml puromycin. 24 to 48 h later all non-transfected cells had detached. Once the plates had reached confluence they were split at a 1 to 3 ratio to finalize the selection. These plates were then allowed to reach confluence and transferred in a 1 to 2 ratio to poly-L-lysine coated plates for harvesting purposes. The next day the adherent cells were washed with PBS and serum free medium was added to the plates (DMEM/F12 1:1 supplemented with 10 mg/l L-Glutathione reduced, 161 mg/l N-Acetyl-L-Cysteine, 1% pen/strep and 1 μg/ml puromycin). Supernatants were collected every 3½ days over a period of 3 to 4 weeks. Cellular debris were removed by centrifugation at 3000 g for 10 minutes followed by filtration through a 0.2μ filter. The Adiponectin containing medium was kept at 4° C. until further processing.

### Example 2

Purification of the Baits

Concentration of FLAG-Tagged Fusion Proteins

In order to get an efficient purification by FLAG affinity chromatography the FLAG tagged proteins had to be concentrated first from the cell culture supernatants. Two different methods have been used, either anion exchange chromatography or ammonium sulfate precipitation. Both methods have successfully been used by others to purify biologically active Acrp30 from cell culture supernatants. The two methods are briefly described bellow.

Anion Exchange Chromatography

Supernatants containing FLAG tagged proteins (Acrp16-FLAG-C (SEQ ID NO: 9), Acrp30-FLAG-C (SEQ ID NO: 8)) were equilibrated to pH 8.0 with 20 mM HEPES and loaded on anion exchange columns (Q-Sepharose fast flow; Pharmacia Cat. No. 17-0510-01) at flow rates of 2-4 ml/min at 4° C. The columns were then washed with at least 10 column volumes of 20 mM HEPES pH 8.0, 50 mM NaCl. Bound proteins were eluted at a flow rate of 4 ml/min on an Akta purifier (Pharmacia) with 20 mM HEPES pH 8.0 with the following gradient: 50 to 500 mM NaCl in 5 column volumes, 500 to 750 mM NaCl in 1 column volume and 1 column volume at 1.5 M NaCl. The fractions were analyzed by SDS-PAGE and coomassie brilliant blue staining or anti FLAG western blot. The Adiponectin containing fractions were pooled and filtered with a 0.2μ filter and stored at 4° C. until FLAG affinity purification.

Ammonium Sulphate Precipitation

Supernatants containing FLAG tagged proteins (Acrp16-FLAG-N (SEQ ID NO: 10)) were equilibrated to pH 8.0 with 20 mM HEPES and 40% w/v ammonium sulfate was added to the supernatants. Ammonium sulfate precipitation was performed at 4° C. under stirring for 6-8 h. The precipitate was then pelleted for 1 h at 8000 g at 4° C. The pellet was resuspended in 20 mM HEPES, pH 8.0 supplemented with 50 mM NaCl. Insoluble material was removed by filtration through a 22 μM Millex GV sterile filters (Millipore) filter and the resuspended precipitate was stored at 4° C. until FLAG affinity purification.

FLAG Affinity Chromatography

All FLAG tagged constructs were purified by affinity chromatography using M2-FLAG-Agarose resin (Sigma, Cat. No. A220) as follows. The concentrated samples were pumped over the FLAG column at room temperature using a peristaltic pump at flow rates of 1-2 ml/min. The columns were then washed with at least 20 column volumes of TBS (10 mM Tris/HCl pH 7.5). Bound proteins were eluted with FLAG peptide (Sigma, Cat. No. F3290) at a concentration of 0.1 mg/ml in TBS. The protein containing fractions were pooled and concentrated using Millipore Ultrafree centrifugal filters 5K (Millipore, Cat. No. UFV4BCC25). The same concentration filters were used to perform a buffer exchange to PBS and to get rid of residual FLAG peptide. An at least 200 fold dilution was performed on the different samples. Purified proteins were sterile filtered using Millipore Millex filters (Millipore Cat. No. SLGV 004 SL) and stored in PBS at 4° C.

Analysis and Quantification of the Baits

The different bait preparations were quantified both, by Bradford analysis (Biorad) according to the manufacturer's recommendation using IgG as a standard and by UV absorption using the theoretical extinction coefficient (ε). The theoretical extinction coefficient was determined on the ExPASy web site. Briefly the samples were diluted 1 to 10 in PBS and the absorption at 280 nm was determined. The concentration was calculated as follows: Dilution×MW/ε×OD280=mg/ml (MW=Molecular Weight, OD=optic density). The results of the quantification are shown in FIG. 4. The samples were then diluted to 1.5 mg/ml based on the UV measurements. The purity of the proteins was assessed by SDS-PAGE and subsequent coomassie staining of the gels. All samples showed a high degree of purity as documented by the appearance of a major band (>90%) in the SDS page analysis. Acrp30-FLAG-C showed the typical reported three bands due to differential glycosylation of the protein.

### Example 3

Construction of an Alphaviral cDNA Expression Library Containing the Adiponectin Receptor

Total RNA was isolated from differentiated C2C12 mouse myoblast cells with the RNeasy™ RNA isolation kit (Qiagen). Selection of polyA+ RNA was carried out with the Oligotex™ mRNA isolation kit (Qiagen). Single-stranded cDNA was produced from 1 μg polyA+ RNA with PowerScript™ reverse transcriptase (Clontech) using the template switch protocol (Zhu et al., 2001), with the 3′-Sfi oligonucleotide (5′-AAG CAG TGG TAT CAA CGC AGA GTG GCC GAG GCG GCC TTT TTT TTT TTT TTT TTT TTT TTT TTT TTT VN-3′) (SEQ ID NO: 11) as primer, and the 5′-Sfi oligonucleotide (5′-d[AAG CAG TGG TAT CAA CGC AGA GTG GCC ATT ACG GCC] r[GGG]-3′) (SEQ ID NO: 12) as switch template.

Double-stranded cDNA was then produced by 14 cycles of polymerase chain reaction (PCR), using the Advantage2 polymerase mix (Clontech) and an anchor primer (5′-AAG CAG TGG TAT CAA CGC AGA GT-3′) (SEQ ID NO: 13) in a total volume of 500 μl. Double-stranded cDNA was purified with the Qiaquick PCR purification kit (Qiagen), digested with the restriction endonuclease Sfi1 (Roche), and size-fractionated by agarose gel electrophoresis. Three fractions, corresponding to large (>3 kb; fraction A), intermediate (1.5-3 kb; fraction B), and small cDNAs (0.4-1.5 kb; fraction C) were isolated by electroelution and cloned separately into the alphaviral expression vector pDelSfi. The sublibraries A, B, and C consisted of 7×106, 2×107, and 1.2×107 independent transformants, respectively. DNA was isolated from pooled colonies using the HiSpeed Plasmid Maxi Kit (Qiagen).

Plasmids were prepared for in vitro transcription as follows. 5 μg of each sublibrary were linearized, half with the restriction endonuclease Not1 (Roche), the other half with Pac1 (New England Biolabs). 5 μg of the helper plasmid pDHEB (Bredenbeek et al., 1993), encoding the Sindbis virus structural proteins, were linearized with the restriction endonuclease EcoR1. All restriction digests were then extracted with phenol-chloroform, ethanol precipitated, and resuspended in RNase-free H2O at a concentration of 0.5 μg/μl. 1 μg of each linearized sublibrary and of the helper plasmid were subjected to SP6 RNA polymerase-mediated in vitro transcription in a volume of 20 μl, using the mMessage mMachine™ kit (Ambion). Each sublibrary RNA was co-electroporated with an equimolar amount of helper RNA into 107 BHK cells. 18 hours post transfection, cell supernatants were harvested and the viral titers determined. The titers were: fraction A, 8×106; fraction B, 1.2×107; fraction C, 1.5×107.

### Example 4

Screening for the Adiponectin Receptor by Fluorescence-Activated Cell Sorting

Subconfluent (80%) baby hamster kidney (BHK) cells were infected with the C2C12 viral library at a multiplicity of infection (MOI) of 0.2. 2×107 cells were infected with each sublibrary. 5.5 hours post-infection cells were detached with cell dissociation buffer (Sigma), washed and stained for 45 min with Acrp30-FLAG-C at a concentration of 50 μg/ml. After extensive washing, cells were incubated with a mouse anti FLAG antibody (FLAG M2, Sigma) at a concentration of 1.8 μg/ml for 30 min. After washing, cells were stained for additional 30 min with FITC-conjugated anti-mouse IgG antibody (Jackson ImmunoResearch) at a concentration of 5 μg/ml. All stains were performed in HP1 medium (TurboDoma medium, Cell Culture Technologies GmbH) supplemented with 0.5% FCS at 4° C.

Cell pools were then filtered and sorting was performed on a FACS Vantage flow cytometer (Becton Dickinson). In a first step FITC-positive cells were enriched and stained with propidium iodide (PI) to exclude dead cells. Afterwards single cell sorting for bait binding (higher FL1 fluorescence) PI negative cells was carried out. Each sorted single cell was incubated in a well of a 24-well plate containing 50% confluent BHK feeder cells. Upon virus spread (2-3 days post-sorting), the infected cells were tested by FACS analysis for Adiponectin binding as described above for the sorting. To exclude false positive events an Fc-receptor (FcR) staining was done in parallel. At day 2, 69 out of 176 wells showed typical signs of viral infection and 43 bound the Adiponectin baits, 4 wells were identified as FcR's. Twenty samples binding Adiponectin were further processed for gene rescue.

### Example 5

Rescue of cDNA Encoding the Adiponectin Receptor, T-Cadherin

To obtain the cDNA encoding a putative Adiponectin receptor, a RT-PCR was performed using 20 supernatants, each containing recombinant Sindbis virus.

For the viral RNA isolation 50 μl of viral supernatant and QIAmp Viral RNA Kit (Qiagen, Cat No.: 52409) was used. The procedure was performed according to manufacturer's protocol and the RNA was dissolved in 30 μl AVE elution buffer.

For cDNA synthesis 9 μl of the viral RNA were reversely transcribed in a total volume of 20 μl at 42° C. for 1 hour using 200 Units SUPERSCRIPT™ II RNase H-reverse transcriptase (Invitrogen Life Technologies, Cat. No. 18064-022), 10 pmol LPP2 primer (5′-ACA AAT TGG ACT AAT CGA TOG C-3′) (SEQ ID NO: 14) according to the manufacturer's protocol. The reaction was terminated by incubation at 70° C. for 15 minutes. To remove the complementary RNA prior to PCR the cDNA was treated with 2 Units of RNase H at 37° C. for 30 minutes.

The PCR was performed using 5 ul cDNA as template, Expand High Fidelity PCR System (Roche, Cat. No. 1 732 650) and the primers GW-Del7630 (5′-GGG GAC AAG TTT GTA CAA AAA AGC AGG CTC TAC AAC ACC ACC ACC TCT AG-3′) (SEQ ID NO: 15) and GW-LPP2 (5′-GGG GAC CAC TTT GTA CAA GAA AGC TGG GTA CAA ATT GGA CTA ATC GAT GGC-3′) (SEQ ID NO: 16). The PCR reaction was performed on an Eppendorf Mastercycler gradient thermal cycler with one predenaturation step of 2 min at 94° C., followed by 34 cycles of 20 sec. at 94° C., 20 sec at 60° C., and 3 min plus 10 sec each cycle at 68° C., and one final cycle of 20 min at 68° C. in a 25 ul reaction volume. The resulting PCR product was analyzed on an agarose gel and isolated using QIAquick PCR purification Kit (Cat No.: 218104). The isolation was performed according to manufacturer's protocol. The PCR product was eluted in 30 μl Elution buffer (10 nM Tris) and directly sequenced with LPP-1 primer (ATACGACTCACTATAGGGAGAC) (SEQ ID NO: 17). Obtained sequences were analyzed for identity or similarity using the standard nucleotide-nucleotide BLAST (blastn) similarity search program and the sequences of GenBank+EMBL+DDBJ+PDB (but no EST, STS, GSS, or phase 0, 1 or 2 HTGS sequences). Analysis of 20 clones revealed that the novel receptor for adiponectin is T-cadherin. Based on these findings the remaining 23 Adiponectin positive clones were analysed by RT-PCR using T-cadherin specific primers T-Cad-1: CAG CCG AGA ACT CCG CTC AC (SEQ ID NO: 18), and T-Cad-2. CGG TGA GCC GGA ACT TOG AC (SEQ ID NO: 19). All clones were shown to be T-cadherin. Hence T-cadherin was cloned 43 times independently as an interaction partner of adiponectin.

### Example 6

Adiponectin-Bait Acrp30-FLAG-C Binds To T-Cadherin in Transiently Transfected 293 Cells

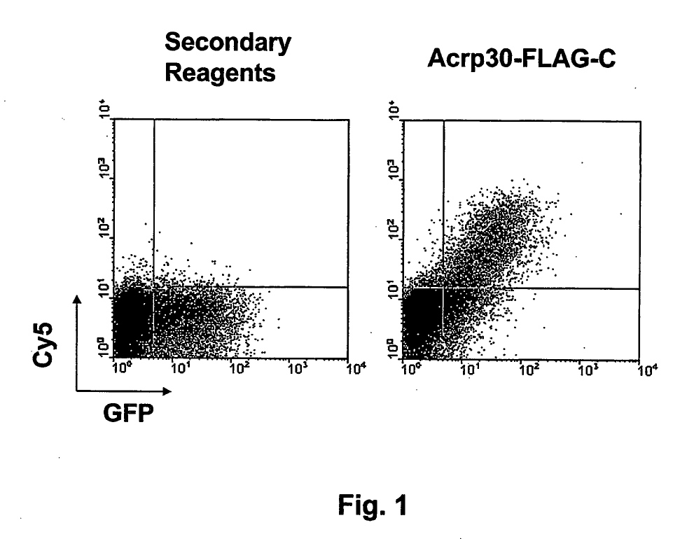

In order to validate the binding of adiponectin to T-cadherin with an independent expression system, T-cadherin was subcloned into a mammalian expression vector. For this purpose a T-cadherin cDNA containing gateway B1 and B2 sites resulting from the gene rescue was used to perform a gateway BP reaction with an appropriate donor vector (pDonor201, Invitrogen) according to the manufacturer's recommendations. The resulting clone was named pEN-T-cadherin. pEN-T-cadherin was then used in a gateway LR reaction according to the manufacturer's recommendation to transfer the cDNA into a mammalian expression vector (pGF-GW). pGF-GW contains a gateway cassette (Invitrogen) downstream of the CMV promoter, such that cDNAs which are recombined with the gateway technology are under the control of the CMV promoter. In addition pGF-GW contains GFP under the control of a retroviral LTR. This vector allows the visualization of transfected cells by flow cytometry and since T-cadherin and GFP are on the same vector, all the cells which are GFP positive also express T-cadherin. 293-EBNA cells were seeded on day previous transfection at a density of 4.5×106 cells per 10 cm dish. The next day the cells were transfected using lipofectamine 2000 according to the manufacturer's recommendation (Invitrogen). Two days after transfection the cells were harvested and stained with Adiponectin. Briefly the cells were incubated with 50 μg/ml purified Acrp30-FLAG-C for 30 minutes, followed by incubation with mouse anti-FLAG (M2, Sigma, 2 μg/ml, 30 minutes), and Cy5-coupled anti-mouse (Jackson, 1 μg/ml). Between the different incubation steps the samples were extensively washed. All stains and washes were performed with PBS supplemented with 1% FCS. The stained samples were than analysed by FACS using a FACS Calibur (Becton Dickinson). As shown in FIG. 1 Acrp30-FLAG-C showed clear binding to the GFP positive population in the cells transfected with pGF-T-cadherin, whereas no binding was detected to the GFP negative (untransfected) cells. The secondary reagents alone neither stained the GFP negative nor the GFP positive cell population. Similarly no binding of Acrp30-FLAG-C was detected to cells transfected with empty vector (pGF-GW). These data confirm with an independent expression system that T-cadherin specifically binds to adiponectin and that T-cadherin is therefore a novel adiponectin receptor.

### Example 7

Determination of the Dissociation Constant of the Adiponectin-T-Cadherin Interaction

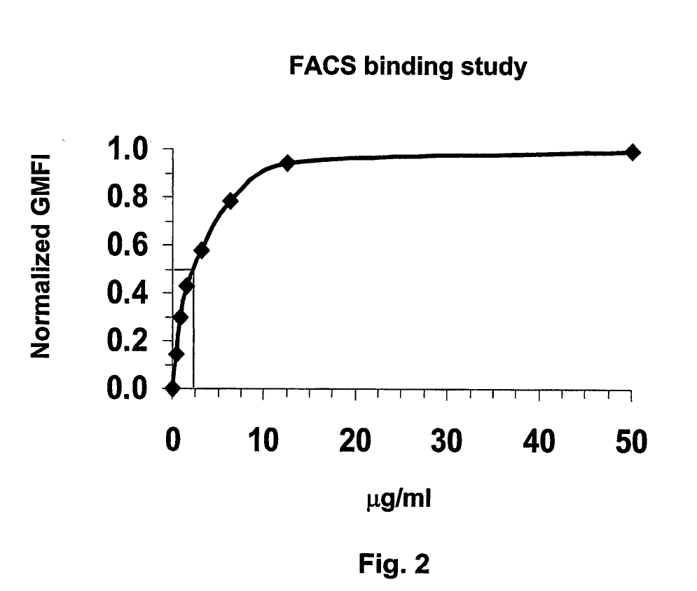

In order to determine the dissociation constant of the adiponectin-T-cadherin interaction, 293-EBNA cells were transfected with pGF-T-cadherin as described in example 6. Two days after transfection the cells were harvested and stained with decreasing concentration of Acrp30-FLAG-C (50, 12.5, 6.3, 3.1, 1.6, 0.8, 0.4 and 0 μg/ml) as described in example 6. The cells were then analysed by FACS using a FACS Calibur (Becton Dickinson). To determine the binding affinity, the geometric mean fluorescence of the GFP positive population was determined using FACS WINmdi software. The geometric mean fluorescence was then normalised by deducing the mean fluorescence of the sample which was stained with the secondary reagents only from the different samples and then all the values were divided by the maximal fluorescence. The normalised values for the mean fluorescence were than plotted against the concentration of Acrp30-FLAG-C. As shown in FIG. 2 the binding is saturated at the highest concentrations of Acrp30-FLAG-C and gradually declines with decreasing amounts of protein. The concentration for half maximal binding was determined as 2.2 μg/ml. The Kd was then calculated by dividing the concentration for half maximal binding (0.0022 g/l) by the molecular weight of the protein (26546 g/Mol) leading to a Kd of 83 nM. This Kd is in accordance with a physiological role of the T-cadherin-adiponectin interaction, since the circulating concentrations of adiponectin is in the range of 10 μg/ml in the serum of healthy individuals.

### Example 8

Identification of an Inhibitor of the Adiponectin-T-Cadherin Interaction

Methods for the identification of small molecule inhibitors are well known in the art (Comb Chem High Throughput Screen. 1998 December; 1(4):171-83. Review.). Cell free systems may be most useful, where adiponectin or T-cadherin is conjugated to a solid phase and before soluble T-cadherin or adiponectin, respectively, is added in the presence of various small molecule. Small molecules are looked for that block the interaction between adiponectin and T-cadherin. Preferably, thousands or hundreds of thousands assays are run in parallel using one small molecule per assay.

Alternatively, a cell line expressing T-cadherin may be used and the interaction between the adiponectin and the cell line is blocked using small molecules in an assay similar to the one mentioned above.

There are various ways to measure the interaction between two proteins that are well known in the art (Comb Chem High Throughput Screen. 1998 December; 1(4):171-83. Review.)

### Example 9

Identification of an Inhibitor of the Adiponectin-T-Cadherin Interaction: Monoclonal Antibody

Animals, preferably mice and more preferably mice with a humanized B cell repertoire are immunized with the extracelluar part of T-cadherin coupled to a carrier, preferably a protein carrier. Alternatively, mice may be immunized with DNA encoding T-cadherin, preferably linked to a T helper cell epitope. Alternatively, mice may be immunized with fragments of T-cadherin. Monoclonal antibodies are generated thereafter using standard methods (see e.g. Chapter 6, Antibodies A Laboratory Manual, Cold Spring Harbor Laboratory Press, 1988). Monoclonal antibodies may then be selected for their ability to block the adiponectin T cadherin interaction, characterizing them as antagonists. Alternatively, monoclonal antibodies specific for T-cadherin may be generated using phage display methods which are well known in the art (see e.g. Azzazy et al. 2002, Clin Biochem. 35(6):425-45). The specific monoclonal antibodies may then be characterized for their ability to block the adiponectin T-cadherin interaction.

### Example 10

Identification of a T-Cadherin Agonist

Methods for the identification of small molecule agonists are well known in the art (Comb Chem High Throughput Screen. 1998 December; 1(4):171-83.). Cell based systems may be most useful for the task. Specifically, a cell line may be generated that is transfected with T-cadherin thereby becoming responsive to the treatment with adiponectin. The parental cell line is not responsive to adiponectin. In a high-throughput screening assay, small molecules are now looked for that trigger a adiponectin-like response in the transfected cell line but not in the parental cell line.

### Example 11

Identification of a T-Cadherin Agonist: Monoclonal Antibody

Animals, preferably mice or rats and more preferably mice with a humanized B cell repertoire are immunized with the extracelluar part of T-cadherin coupled to a carrier, preferably a protein carrier. Alternatively, mice may be immunized with DNA encoding T-cadherin, preferably linked to a T helper cell epitope. Alternatively, mice may be immunized with fragments of T-cadherin. Monoclonal antibodies are generated thereafter using standard methods (see e.g. Chapter 6, Antibodies A Laboratory Manual, Cold Spring Harbor Laboratory Press, 1988). Monoclonal antibodies may then be selected for their ability to mimic the action of adiponectin in vivo or in vitro, characterizing them as T-cadherin agonists. Alternatively, monoclonal antibodies specific for T-cadherin may be generated using phage display methods which are well known in the art (see e.g. Azzazy et al. 2002, Clin Biochem. 35(6):425-45). The specific monoclonal antibodies may then be selected for their ability to mimic the action of adiponectin in vivo or in vitro, characterizing them ad T-cadherin agonists.

### Example 12

Method for Determining Whether an Agent that Interacts with T-Cadherin Improves Insulin Sensitivity In Vitro

Adiponectin has been shown to enhance insulin activity. Therefore, molecules that interact with the adiponectin receptor (T-cadherin) likely function to improve insulin sensitivity. Agents that interact with T-cadherin, such as, e.g., antibodies or other chemical agents, are tested for their ability to improve insulin sensitivity using in vitro assays. An exemplary assay is described in Berg et al., Nat. Med. 7:947-953 (2001). Briefly, single cell suspensions of hepatocytes are obtained from perfusions of Sprague-Dawley rats using the procedure of Berry and Friend, J. Cell. Biol. 43:506-520 (1969), and the perfusion method of Leffert et al, Methods in Enzymol. 58:536-544 (1979). The cells are plated on tissue culture plastic for 6 h at a density of 2×105 cells per well in a 24 well plate which has been pre-coated with rat-tail collagen I. This can be down scaled to a 96 well format, adapting the volumes and numbers of cells according to the cell surface. During plating, cells are cultured in RPMI 1640 medium supplemented with 10% FCS, penicillin/streptomycin 10 μg/ml insulin and 10 μm dexamethasone.

After the cells have adhered to the plates the media is changed to RPMI 1640 medium supplemented with 5 mM glucose, 0.4% FCS in the absence of insulin and dexamethasone. The cells are then allowed to adapt to this low glucose medium over night.

The following morning, the medium is refreshed and sub-physiological concentrations of insulin are added to the hepatocyte cultures in the presence or absence of increasing amounts of a test agent. The test agent is an agent that interacts with T-cadherin such as, e.g., an antibody or a chemical compound that exhibits physical interaction with T-cadherin. If antibodies are used, they are preferably administered at a concentration from about 0 to 10 mg/ml. When the test agent is a chemical compound, it is preferably administered at a concentration of from about 0 to 100 mM. Sub-physiological concentrations of insulin shall mean concentrations of insulin which have no effect on hepatic glucose production. These concentrations are typically in the range of 35 pM but may vary from one insulin batch to another.

To determine the optimal concentration of insulin for this assay, a titration experiment should be performed on every insulin batch. For the titration experiments, typically about 0 to about 1000 pM insulin is used. To test enhancement of insulin action, the highest concentration that does not lead to reduced glucose production is used. This concentration is generally in the range of 35 pM.

The cells are next incubated for another 24 h. Then the cells are incubated in glucose-free medium containing 5 mM each of alanine, valine, glycine, pyruvate and lactate. The glucose production is then measured with a Trinder assay (Sigma). A reduction in glucose production using the test agent, as compared to the glucose production observed when the test agent is not used (or other suitable control assay), will identify the test agent as one that mimics an action of adiponectin.

### Example 13

Method for Determining Whether an Agent that Interacts with T-Cadherin Enhances Insulin-Mediated Glucose Uptake and/or IRS-1-Mediated PI-3-Kinase Activity In Vitro

Adiponectin has been shown to enhance insulin-mediated glucose uptake and to enhance IRS-1-mediated PI-3-kinase activity in differentiated skeletal muscle cells (C2C12). Therefore, molecules that interact with T-cadherin can be assayed for their ability to enhance insulin mediated glucose uptake and/or IRS-1 mediated PI-3-Kinase activity in differentiated C2C12 cells in order to determine if the molecules mimic an action of adiponectin. These assays can be performed as described in Maeda et al., Nat. Med. 8:731-737 (2002) and del Aguila et al., Am. J. Physiol. 276:E849-855 (1999).

Briefly, cultured C2C12 skeletal muscle cells are maintained according to the supplier's instructions (ATCC). To induce differentiation, C2C12 cells are allowed to reach confluence and then are transferred to DMEM supplemented with 2% horse serum. The cells are then incubated for another 5-7 days in this differentiation medium. At this time the myotube formation is maximal. The cells are then incubated in the presence or absence of increasing concentrations of a test agent. The test agent is an agent that interacts with T-cadherin such as, e.g., an antibody or a chemical compound that exhibits physical interaction with T-cadherin. If antibodies are used, they are preferably administered at a concentration from about 0.1 to 10 mg/ml. When the test agent is a chemical compound, it is preferably administered at a concentration of from about 0 to 100 mM. Then the cells are incubated for the indicated times with or without insulin at a concentration of 100 nM.

For the analysis of IRS-1 associated PI-3-kinase activity, the treated cells are incubated with 100 nM insulin for 5 minutes and then collected and lysed. A 1-mg sample of cell lysate is then immunoprecipitated with 4 μg of IRS-1 polyclonal antibodies, rocking overnight at 4° C. A 40-μl sample of slurry protein A-Sepharose is added to the immunoprecipitate for 2 h, and immunocomplexes are obtained by brief centrifugation at 9,000 rpm and washing three times in PBS-1% NP40, two times in 500 mM LiCl-100 mM Tris, pH 7.6, and one time in 10 mM Tris.HCl, pH 7.4, 100 mM NaCl, and 1 mM trans-1,2-diaminoacylclohexane-N,N,N8,N8-tetraacetic acid. The pellets are then spun down one more time and washed in PI 3-kinase adenosine assay buffer (20 mMTris, pH 7.4, 100 mMNaCl, 10 mM MgCl2, 0.5 mM EGTA, and 120 μM adenosine). The final pellet is then resuspended in 40 μl of PI 3-kinase adenosine assay buffer. A 50-μl sample of phosphatidylinositol and phosphatidylserine is dried down in a nitrogen stream and sonicated in 100 μl of 20 mM HEPES-1 mM EDTA, pH 7.4. The lipid mixture is kept on ice, and 5 μl of this mixture (2 μg/μl of phosphatidylinositol) are added to each sample. The solution is mixed by sonication and incubated for 10 min at 30° C. on a heat block. A mixture consisting of 170 μCi of [g-32P]ATP and 280 μM unlabeled ATP is prepared, and the reaction is started by adding 5 μl of this mixture to each sample. After 10 min at 30° C., the reaction is stopped by the addition of 200 μl 1 N HCl to each sample. The phosphatidylinositol 3-phosphate (PI3P) is extracted with 160 μl chloroform-methanol (1:1). The phases are separated by centrifugation, and the lower organic phase is removed and separated by TLC. The radioactivity incorporated into PI3P is determined by phosphorimaging of the TLC plates. An increase in signal using the test agent, as compared to the signal observed when the test agent is not used (or other suitable control assay), will identify the test agent as one that mimics an action of adiponectin.

To determine the enhancement of insulin-mediated glucose uptake, the treated cells (see above) are used in a glucose uptake assay as described in del Aguila et al., Am. J. Physiol. 276:E849-855 (1999). Briefly, glucose uptake is assayed using 2-DG. After 5 h of serum starvation (the last 5 h of the 24 h treatment with the test agent), cells are incubated with or without insulin (100 nM) for 30 min. The cells are then washed two times with wash buffer (20 mM HEPES, pH 7.4, 140 mM NaCl, 5 mM KCl, 2.5 mM MgSO4, and 1 mM CaCl2). Cells are then incubated in buffer transport solution (wash buffer containing 0.5 mCi 2-[3H]DG/ml and 10 mM 2-DG) for 10 min. Uptake is terminated by aspiration of the solution. Cells are then washed three times, and radioactivity associated with the cells is determined by cell lysis in 0.05 M NaOH, followed by scintillation counting. Aliquots of cell lysates are used for protein content determination. 2-DG uptake is expressed as picomoles per minute per milligram of protein. An increase in signal using the test agent, as compared to the signal observed when the test agent is not used (or other suitable control assay), will identify the test agent as one that mimics an action of adiponectin.

### Example 14

Method for Determining Whether an Agent that Interacts with T-Cadherin Enhances β-Oxidation In Vitro

Adiponectin has been shown to be strong enhancer of β-oxidation in isolated muscle cells. Therefore, molecules that interact with T-cadherin are likely candidates for agents that cause increased fatty acid oxidation in muscle cells. Mouse myoblasts (C2C12 cells) can be used to assay molecules that interact with T-cadherin for the ability to enhance fatty acid oxidation in muscle cells. An assay for fatty acid oxidation is described in Fruebis et al., Proc. Natl. Acad. Sci. USA 13:2005-2010 (2001).

Briefly, cultured C2C12 skeletal muscle cells are maintained according to the supplier's instructions (ATCC). To induce differentiation, C2C12 cells are allowed to reach confluence and then are transferred to DMEM supplemented with 2% horse serum. The cells are then incubated for another 7 days in this differentiation medium. At this time, the myotube formation is maximal. 1 hour before the experiment, the medium is removed and the preincubation (MEM, 3 mM glucose, 4 mM glutamine, 25 mM Hepes, 1% Free fatty acid free BSA, 0.25 mM oleate) medium is added in the presence or absence of increasing concentrations of a test agent. The test agent is an agent that interacts with T-cadherin such as, e.g., an antibody or a chemical compound that exhibits physical interaction with T-cadherin. If antibodies are used, they are preferably administered at a concentration from about 0.1 to 10 mg/ml. When the test agent is a chemical compound, it is preferably administered at a concentration of from about 0 to 100 mM.

Next, [1-C14] oleic acid (1 μCi/ml, American Radiolabeled Chemicals) is added and the cells are incubated for 90 minutes at 37° C. After the incubation period, the medium is removed and assayed for 14CO2 by liquid scintillation counting. An increase in signal using the test agent, as compared to the signal observed when the test agent is not used (or other suitable control assay), will identify the test agent as one that mimics an action of adiponectin.

### Example 15

Method for Determining Whether an Agent that Interacts with T-Cadherin Stimulates AMPK Phosphorylation, ACC Phosphorylation, and/or Lactate Production In Vitro

Adiponectin has been shown to stimulate phosphorylation and activation of 5′-AMP-activated protein kinase and the phosphorylation of acetyl coenzyme A carboxylase (ACC) in the liver and in skeletal muscle. In parallel, adiponectin was shown to stimulate fatty acid oxidation and lactate production in myocytes and to the reduction of molecules involved in gluconeogenesis in the liver. These effects of adiponectin can be used to determine whether an agent that interacts with T-cadherin mimics an activity of adiponectin. The assessment of these activities can be performed as described previously by Yamauchi et al., Nat. Med. 8:1288-1295 (2002).

Briefly, cultured C2C12 skeletal muscle cells are maintained according to the supplier's instructions (ATCC). To induce differentiation, C2C12 cells are allowed to reach confluence and then transferred to DMEM supplemented with 2% horse serum. The cells are then incubated for another 5-7 days in this differentiation medium. At this time the myotube formation is maximal. The cells are then incubated in the presence or absence of increasing concentrations of a test agent. The test agent is an agent that interacts with T-cadherin such as, e.g., an antibody or a chemical compound that exhibits physical interaction with T-cadherin. If antibodies are used, they are preferably administered at a concentration from about 0.1 to 10 mg/ml. When the test agent is a chemical compound, it is preferably administered at a concentration of from about 0 to 100 mM.

The phosphorylation state of ACC and AMPK is then monitored in short intervals from the time of addition of the test agent up to 1 hour after the treatment. The phosphorylation state of these two proteins is assessed by immunoblot analysis using phosphor specific antibodies to AMPK (Cell Signaling) and ACC (Upstate Biotech). Briefly, the cells are harvested and lysed. The concentration of the proteins in the different samples is then determined and equal amounts of the proteins are loaded on a polyacrylamide gel. The proteins are then transferred on nitrocellulose by western blotting. The amount of phosphorylated ACC and AMPK respectively is then assessed using phosphopeptide specific antibodies to ACC and AMPK and the appropriate secondary reagents. Similarly the enzyme activity of AMPK and ACC is measured as described in Minokoshi et al., Nature 415:339-343 (2002). Briefly, to measure the isoform AMPK specific activity in C2C12 cells, AMPK is immunoprecipitated from 100 μg of cell lysate with specific antibodies against AMPK coupled to protein A or protein G sepharose beads. The kinase activity is then measured using a synthetic peptide (SAMS) and [□-32P]ATP. The activity of ACC in C2C12 cell lysates is measured by 14C02 fixation to acid-stable products in the presence or absence of 2 mM citrate, an allosteric activator of ACC. An increase in signal using the test agent, as compared to the signal observed when the test agent is not used (or other suitable control assay), will identify the test agent as one that mimics an action of adiponectin.

To test the activity of T-cadherin antibodies or chemical agents on d lactate production in differentiated C2C12 cells, the cells are differentiated as described above and incubated in the presence or absence of increasing concentrations of a test agent. The test agent is an agent that interacts with T-cadherin such as, e.g., an antibody or a chemical compound that exhibits physical interaction with T-cadherin. If antibodies are used, they are preferably administered at a concentration from about 0.1 to 10 mg/ml. When the test agent is a chemical compound, it is preferably administered at a concentration of from about 0 to 100 mM. The cells are then incubated for 1 h and lactate production is measured after 0, 15, 30 and 60 minutes of incubation. The lactate concentration is determined using a calorimetric method (Lactate C; Wako Pure Chemical Industris, Osaka Japan). An increase in lactate concentration using the test agent, as compared to the lactate concentration observed when the test agent is not used (or other suitable control assay), will identify the test agent as one that mimics an action of adiponectin.

### Example 16

Method for Determining Whether an Agent that Interacts with T-Cadherin Inhibits Smooth Muscle Cell Proliferation In Vitro

The hyperproliferation of smooth muscle cells is implicated in intimal thickening and stenosis. This is especially observed after angioplastiy or generally after artery injury. Adiponectin was shown to reduce smooth muscle cell proliferation. Therefore, molecules that interact with T-cadherin can be assayed for their ability to reduce smooth muscle cell proliferation, thereby identifying such molecules as agents that are useful in the treatment of coronary artery disease.

The ability of agents that interact with T-cadherin to effect smooth muscle cell proliferation can be tested as described in Matsuda et al., J. Biol. Chem. 277:37487-37491 (2002). Briefly, the experiments are performed with human aortic smooth muscle cells (HASMC's) which can be obtained from Clonetics, CA, USA and are maintained in plastic plates precoated with type I collagen (Beckton Dickinson). They are typically used at passage 4 or 5. For cell proliferation assays, HASMC's are treated for 18 hours in Dulbecco modified Eagle Medium containing 2% fetal calf serum with 10 ng/ml human recombinant platelet derived growth factor (PDGF)-BB, HB-EGF, basic fibroblast growth factor (FGF), epidermal growth factor (EGF) in the presence or absence of increasing concentrations of a test agent. The test agent is an agent that interacts with T-cadherin such as, e.g., an antibody or a chemical compound that exhibits physical interaction with T-cadherin. If antibodies are used, they are preferably administered at a concentration from about 0.1 to 10 mg/ml. When the test agent is a chemical compound, it is preferably administered at a concentration of from about 0 to 100 mM.

A proliferation assay is then conducted. This assay generally involves evaluating the incorporation of radioactively labeled thymidine. Typically, cells are exposed to [H3] thymidine (Amersham, Pharmacia Biotech) at 20 μCi/ml for 6 hours, then trypsinised and retrieved onto glass fiber filter using an automated cell harvester. [H3] thymidine uptake, a measure for proliferation rates, is then measured in a direct beta counter. A decrease in [H3] thymidine uptake using the test agent, as compared to the [H3] thymidine uptake observed when the test agent is not used (or other suitable control assay), will identify the test agent as one that mimics an action of adiponectin. Test agents that are shown to mimic an activity of adiponectin by this assay can be tested further using in vivo assays such as those described elsewhere herein.

### Example 17

Method for Determining Whether an Agent that Interacts with T-Cadherin Inhibits Smooth Muscle Cell Migration In Vitro

Smooth muscle cell migration is observed after vascular trauma, for example, trauma caused during angioplasty, which frequently leads to stenosis. Adiponectin has been shown to inhibit smooth muscle cell migration. Therefore, agents that interact with T-cadherin can be tested for their ability to inhibit smooth muscle cell migration in order to determine if such molecules mimic an action of adiponectin.

Exemplary assays for smooth muscle cell migration are described in Matsuda et al., J. Biol. Chem. 277:37487-3791 (2002). Human aortic smooth muscle cells (HASMC's), which can be obtained from Clonetics, CA, USA, are maintained in plastic plates precoated with type I collagen (Beckton Dickinson). HASMC's are used at passage 4 to 5. In order to assess the ability of test agents to influence the migration of these cells, Boyden chambers can be used. HASMC's are added to the transwell inserts (Coster, 12 mm diameter, 12 μm pore size) at a density of 5×104 cells per ml. Migration of the HASMC's is induced by the addition of HB-EGF at a concentration of 10 ng/ml in the presence or absence of increasing concentrations of a test agent. The test agent is an agent that interacts with T-cadherin such as, e.g., an antibody or a chemical compound that exhibits physical interaction with T-cadherin. If antibodies are used, they are preferably administered at a concentration from about 0.1 to 10 mg/ml. When the test agent is a chemical compound, it is preferably administered at a concentration of from about 0 to 100 mM.

To monitor the migration activity of HASMC's, the transwell chambers are incubated for 4 hours under culture conditions. Migrated HASMC's on the lower surface of the membrane are then fixed with ethanol and stained with heamatoxylin. The extent of migration is then monitored microscopically by counting the number of stained nuclei per high power field (HPF; ×400).

To measure the migration of HASMC's in response to growth factors in a high throughput format 96 well plates with a cover plate that contains membranes at the bottom can be used (Millipore, Multiscreen-Migration, Invasion and Chemotaxis Multiscreen 96-well plate with tray). Typically, the HASMC's are added to the top plate and the migration inducing HB-EGF at a concentration of 10 ng/ml is added to the bottom well. Test agents are then either added in the top or the bottom well. The plates are then incubated for a defined time, typically, a time between 1 and 12 h under cultivation conditions. The amount of migrated cells (cells in the bottom well) can then be monitored by colorimetric, radioactive, or microscopic methods. Alternatively the migrated cells can be counted by a fluorescence activated cell sorter.

A decrease in migrated cells using the test agent, as compared to the migrated cells observed when the test agent is not used (or other suitable control assay), will identify the test agent as one that mimics an action of adiponectin. Test agents that are shown to mimic an activity of adiponectin by this assay can be tested further using in vivo assays such as those described elsewhere herein.

### Example 18

Method for Determining Whether an Agent that Interacts with T-Cadherin Inhibits NF-κB Signaling or Stimulates cAMP Levels In Vitro

Adiponectin has been shown to inhibit TNF-α induced NF-κB signaling through a cAMP dependent pathway in human aortic endothelial cells (HAEC's). Therefore, the ability of molecules that interact with T-cadherin to inhibit TNF-α induced NF-κB signaling identifies such molecules as those that mimic an activity of adiponectin. NF-κB signaling can be monitored, for example, by the method of Ouchi et al., Circulation 102:1296-1301 (2000).

Briefly, HAECs (Clonetics) are maintained in plastic plates precoated with type I collagen (Becton Dickinson). To address the effect of agents on TNF-α induced NF-κB signaling, HAECs in a confluent state are preincubated for 18 hours in medium 199 (Gibco) containing 0.5% FCS and 3% BSA with increasing amounts of a test agent. The test agent is an agent that interacts with T-cadherin such as, e.g., an antibody or a chemical compound that exhibits physical interaction with T-cadherin. If antibodies are used, they are preferably administered at a concentration from about 0.1 to 10 mg/ml. When the test agent is a chemical compound, it is preferably administered at a concentration of from about 0 to 100 mM.

The cells are then exposed to human recombinant TNF-α (R&D systems) or vehicle at a final concentration of 10 U/mL for the times indicated. To measure IκB-α phosphorylation, the proteosome inhibitor MG132 (20 μmol/l) is added to the cells 1 hour before TNF-α to stabilize the phosphorylated form of IκB-α. Then, TNF-α is added to the cells (10 U/ml). The phosphorylation status of IκB-α is then determined by immunoblot analysis. As a loading control an antibody against GAPDH is used. The cells are collected 0, 30 and 60 minutes after the addition of TNF-α. Whole-cell lysates are then resolved on 12.5% SDS-PAGE gels, followed by electrophoretic transfer to nitrocellulose membranes (Amersham). The membranes is then exposed to primary antibodies (anti-phospho-specific IκB-α Ser32 from New England Biolabs and anti GAPDH from Biogenesis), and is then exposed to secondary antibodies conjugated to HRP. The antibody is detected with an ECL Western Detection Kit (Amersham). A decrease in signal using the test agent, as compared to the signal observed when the test agent is not used (or other suitable control assay), will identify the test agent as one that mimics an action of adiponectin.

Similarly, since adiponectin has been shown to lead to increased cAMP levels in HAEC's, test agents can also be assessed for their ability to increase the cAMP levels in HAECs in order to determine if they mimic an activity of adiponectin. cAMP levels can be assayed, for example, by the method of Ouchi et al., Circulation 102:1296-1301 (2000).

Briefly, HAECs (2×1 05 cells/well in 24-well plates) are stimulated with increasing amounts of a test agent in medium 199 containing 0.5% FCS and 3% BSA for 18 hours. Dishes are then placed on ice, and media is changed to ice-cold PBS to terminate the reaction. Intracellular cAMP is then determined with an enzyme immunoassay kit (Amersham) according to the manufacturer's instructions. An increase in signal using the test agent, as compared to the signal observed when the test agent is not used (or other suitable control assay), will identify the test agent as one that mimics an action of adiponectin.

### Example 19

Method for Determining Whether an Agent that Interacts with T-Cadherin Inhibits Endothelial Adhesion Molecule Expression In Vitro

Adiponectin has been shown to inhibit TNF-α induced cell surface expression of vascular cell adhesion molecule-1 (VCAM-1), endothelial-leukocyte adhesion molecule-1 (E-selectin), and intracellular adhesion molecule-1 (ICAM-1) in human aortic endothelial cells (HAEC's). Therefore, molecules that interact with T-cadherin can be tested for their ability to inhibit the expression of these adhesion molecules in order to determine if they mimic an activity of adiponectin. Methods for assessing cell surface expression of adhesion molecules are described, for example, in Ouchi et al., Circulation 100:2473-2476 (1999).

Briefly, HAECs (Clonetics) are maintained in MCDB131 medium (Clonetics) supplemented with 5% FCS, and cells from passages 4 through 6 are used for experiments. HAECs cultivated in collagen type I coated plates are allowed to reach confluence. and incubated for 18 hours in medium 199 (Gibco) containing 0.5% FCS and 3% BSA with increasing amounts of a test agent. The test agent is an agent that interacts with T-cadherin such as, e.g., an antibody or a chemical compound that exhibits physical interaction with T-cadherin. If antibodies are used, they are preferably administered at a concentration from about 0.1 to 10 mg/ml. When the test agent is a chemical compound, it is preferably administered at a concentration of from about 0 to 100 mM.

The cells are then exposed to human recombinant TNF-α (R&D systems) or vehicle at a final concentration of 10 U/mL for 6 hours. The surface expression of VCAM-1, E-selectin and ICAM-1 is then measured by cell ELISA. Briefly, HAECs are incubated at room temperature with anti-human VCAM-1, anti-E-selectin, or anti-ICAM-1 monoclonal antibody (DAKO) at 1:1000 dilution in medium 199 containing 0.5% BSA for 1 hour and then with horseradish peroxidase-conjugated goat anti-mouse IgG (Cappel) at 1:1000 dilution in the same medium. Color formation with o-phenylenediamine dihydrochloride is then measured at 492 nm. A decrease in signal using the test agent, as compared to the signal observed when the test agent is not used (or other suitable control assay), will identify the test agent as one that mimics an action of adiponectin.

### Example 20

Method for Determining Whether an Agent that Interacts with T-Cadherin Prevents Neointimal Thickening after Vascular Injury In Vivo

Adiponectin has been shown to reduce neointimal thickening in animal models following vascular injury. Therefore, molecules that interact with T-cadherin can be assayed for their ability to prevent neointimal thickening after vascular injury in order to identify the molecules as those which mimic an activity of adiponectin. Exemplary methods for assessing neointimal thickening are described in Matsuda et al., J. Biol. Chem. 277:37487-3791 (2002).

Briefly, femoral artery injury is induced in mice by a straight spring wire (0.36 mm in diameter, No. SKI 175 FLP 14-S, INVATEC, Concessio (BS), Italy) denuding vascular endothelium and inducing neointimal hyperplasia. A test agent is administered to the animals. The test agent is an agent that interacts with T-cadherin such as, e.g., an antibody or a chemical compound that exhibits physical interaction with T-cadherin. Preferably, the test agent is an agent that has been shown by in vitro assay (see, e.g., above) to mimic an activity of adiponectin. If antibodies are used, they are preferably administered at a concentration from about 0.1 to 10 mg per injection. When the test agent is a chemical compound, it is preferably administered at a concentration of from about 0 to 100 mm.

The injection protocol is either started one to 5 days before the surgical intervention, at the time of the surgical intervention, or 1 to 10 days after the surgical intervention. Typically, a single administration of test compound is given to the animals, or several injections (up to 10 injections per day) are given to the animals. Alternatively, the mice are treated by continuous infusion of the test compound, starting the infusion 1-5 days previous to the surgical intervention, at the day of the surgical intervention or 1-10 days after the surgical intervention. For the constant delivery of the test agent, an osmotic pump (Alzet, Newark, Del.) is implanted which delivers the required daily amounts of molecule. For injection studies the mice are either injected intravenously or intraperitonealy with the indicated amounts of test agent.

Similarly, the mice can be treated by local administration of the test agent at the indicated concentrations by means of injection at the place of surgery. As controls, the animals are treated with either PBS or with a control molecule (e.g., an unrelated antibody generated in the same species (isotype control)).

To evaluate the beneficial effects of the treatment, 2-3 weeks after vascular injury the mice are anesthetized and both femoral arteries are harvested after perfusion, fixed with 10% formalin and embedded in paraffin. After embedding in paraffin, parallel sections are stained with hematoxylin and eosin. Smooth muscle cells are identified by immunostaining for α-smooth muscle actin using clone 1A4 from Sigma as the primary antibody. Intimal and Medial area are than measured using image analysis software MacSCOPE.

A reduction in neointimal thickening after administration of the test agent, as compared to the neointimal thickening observed when the test agent is not used (or other suitable control assay), will identify the test agent as one that mimics an action of adiponectin.

### Example 21

Method for Determining Whether an Agent that Interacts with T-Cadherin Reduces the Development of Coronary Artery Disease In Vivo

Adiponectin has been shown to have preventative effects against the development of coronary artery disease (CAD) in mouse models. Therefore, molecules that interact with T-cadherin can be tested for their ability to prevent CAD in order to identify the molecules as those that mimic an activity of adiponectin. One model for the study of the development of coronary artery disease are Apo E deficient mice, a well established model for arthroscleroses. These mice are hypercholesterolemic and spontaneously develop severe arthroscleroses.

Molecules that interact with T-cadherin are tested for their ability to prevent arterioscleroses in Apo E deficient mice. A test agent is administered to the animals. The test agent is an agent that interacts with T-cadherin such as, e.g., an antibody or a chemical compound that exhibits physical interaction with T-cadherin. Preferably, the test agent is an agent that has been shown by in vitro assay (see, e.g., above) to mimic an activity of adiponectin. If antibodies are used, they are preferably administered at a concentration from about 0.1 to 10 mg per injection. When the test agent is a chemical compound, it is preferably administered at a concentration of from about 0 to 100 mM. These experiments can be performed, for example, as described in Yamauchi et al., Nat. Med. 8:1288-1295 (2002).

Briefly, a single administration of test agent is given to the animals. Alternatively, the mice are treated by continuous infusion of the test agent. For the constant delivery of the test agent, an osmotic pump (Alzet, Newark, Del.) is implanted as described previously (Fruebis et al., Proc. Natl. Acad. Sci. USA 13:2005-2010 (2001)), which delivers the required daily amounts of test agent. For injection studies, the mice are either injected intravenously or intraperitonaly with the indicated amounts of test agent. As controls, the animals are treated with either PBS or with a control molecule (e.g., an unrelated antibody generated in the same species (isotype control)).

To evaluate the beneficial effects of the treatment, the animals are sacrificed and the aortic atherosclerotic lesions are analyzed after various time points. To monitor the lesions, en face Sudan IV staining of the excised aortas form the arch of the common iliac level is performed after fixation in phosphate buffered 10% formaldehyde. The percentage of Sudan IV-positive areas to total aortic areas is calculated. Quantitative analysis is performed by computer-assisted planimetry. Immunohistochemical analysis and quantification of the atherosclerotic lesions is performed as follows. Briefly, the QCT-embedded, frozen aortic valves are sectioned serially at 10 μm thickness for a total of 300 μm beginning at the base of the aortic valve, where all three leaflets are first visible. Every forth section for a total of five sections from each animal is stained with Oil-Red O to identify the lipid rich lesions. The mouse aortic valve lesions are analyzed immuonohistochemically with the following antibodies: anti-mouse macrophage Mac-3 (Pharmingen) and anti-mouse SRA 2F8 (Serotec). To determine the proportion of SRA-positive macrophages for each animal, the total number of cells positive for Mac-3 or SRA in atherosclerotic plaques of the aorta is counted for each section.

A reduction in Oil-Red-O, Mac-3 or SRA staining after administration of the test agent, as compared to the staining observed when the test agent is not used (or other suitable control assay), will identify the test agent as one that mimics an action of adiponectin.

### Example 22

Method for Determining Whether an Agent that Interacts with T-Cadherin Improves Blood Glucose Levels In Vivo

Adiponectin has been shown to have an insulin sensitizing effect and to improve blood glucose levels in animals. Therefore, molecules that interact with T-cadherin can be tested for their ability to exert an insulin sensitizing effect and/or to improve blood glucose levels in animals in order to identify the molecules as those that mimic an activity of adiponectin. Exemplary methods for assaying blood glucose levels are described in Berg et al., Nat. Med. 7:947-953 (2001).

Briefly, a test agent is administered to either wildtype mice, obese (ob/ob) mice or NOD mice. The test agent is an agent that interacts with T-cadherin such as, e.g., an antibody or a chemical compound that exhibits physical interaction with T-cadherin. Preferably, the test agent is an agent that has been shown by in vitro assay (see, e.g., above) to mimic an activity of adiponectin. If antibodies are used, they are preferably administered at a concentration from about 0.1 to 10 mg per injection. When the test agent is a chemical compound, it is preferably administered at a concentration of from about 0 to 100 mM.

Typically, a single administration of test agent at the doses indicated above is given to the animals or several injections (up to 10 injections per day) are given to the animals. Alternatively, the mice are treated by continuous infusion of the test agent. For the constant delivery of the test agent, an osmotic pump (Alzet, Newark, Del.) is implanted (as described in Fruebis et al., Proc. Natl. Acad. Sci. USA 13:2005-2010 (2001)) which delivers the required daily amounts of test agent. For injection studies, the mice are either injected intravenously or intraperitonealy with the indicated amounts of test agents. As controls, the animals are treated with either PBS or with a control molecule (e.g., an unrelated antibody generated in the same species (isotype control)).

Blood glucose, levels are determined at different time points as described in Berg et al., Nat. Med. 7:947-953 (2001). Briefly, the blood glucose level is measured using a Precision Q-I-D glucose meter (Medisence, Abbott, Chicago). The blood glucose levels are determined either over a time period of day after the first administration or over a longer time period in regular intervals.

A reduction in blood glucose level after administration of the test agent, as compared to the blood glucose level observed when the test agent is not used (or other suitable control assay), will identify the test agent as one that mimics an action of adiponectin. The reduction will be most pronounced when ob/ob or NOD mice are used because these mice have very high glucose levels (250-500 mg/dl). Nevertheless, at least a transient reduction in glucose level will be observed in wild-type mice after administration of an agent that mimics an action of adiponectin (Berg et al., Nat. Med. 7:947-953 (2001)).

### Example 23

Method for Determining Whether an Agent that Interacts with T-Cadherin Causes a Weight Reduction or a Decrease in Triglyceride or Free Fatty Acid Concentration In Vivo

Adiponectin has been reported to reduce the elevated levels of plasma free fatty acids in mice under high fat diet and to lead to a sustainable weight reduction without affecting food intake in mice. Therefore, molecules that interact with T-cadherin can be tested for their ability to reduce the free fatty acid concentration in animals in order to determine if the molecules mimic an activity of adiponectin. Exemplary methods for assaying the effect of an agent on weight reduction and plasma free fatty acid levels are described in Fruebis et al., Proc. Natl. Acad. Sci. USA 13:2005-2010 (2001).

Briefly, a test agent is administered to mice on high fat diet. The test agent is an agent that interacts with T-cadherin such as, e.g., an antibody or a chemical compound that exhibits physical interaction with T-cadherin. Preferably, the test agent is an agent that has been shown by in vitro assay (see, e.g., above) to mimic an activity of adiponectin. If antibodies are used, they are preferably administered at a concentration from about 0.1 to 10 mg per injection. When the test agent is a chemical compound, it is preferably administered at a concentration of from about 0 to 100 mM.

Typically, a single administration of test agent at the doses indicated above is given to the animals or several injections (up to 10 injections per day) are given to the animals. Alternatively, the mice are treated by continuous infusion of the test agent. For the constant delivery of the test agent, an osmotic pump (Alzet, Newark, Del.) is implanted which delivers the required daily amounts of antibody. For injection studies, the mice are either injected intravenously or intraperitonealy with the indicated amounts of test agent. As controls, the animals are treated with either PBS or with a control molecule (e.g., an unrelated antibody generated in the same species (isotype control)).

The mice are weighed at different time points after the treatment in order to determine weight loss. The plasma concentration of triglycerides and free fatty acids are determined with commercial kits (Tryglycerides, Sigma; free fatty acids Wako Biochemicals, Osaka). A reduction in weight, triglyceride and/or fatty acid level after administration of the test agent, as compared to the weight, triglyceride and/or fatty acid level observed when the test agent is not used (or other suitable control assay), will identify the test agent as one that mimics an action of adiponectin.

### Example 24

Reduction of Blood Glucose Levels by a T-Cadherin Specific Antibody

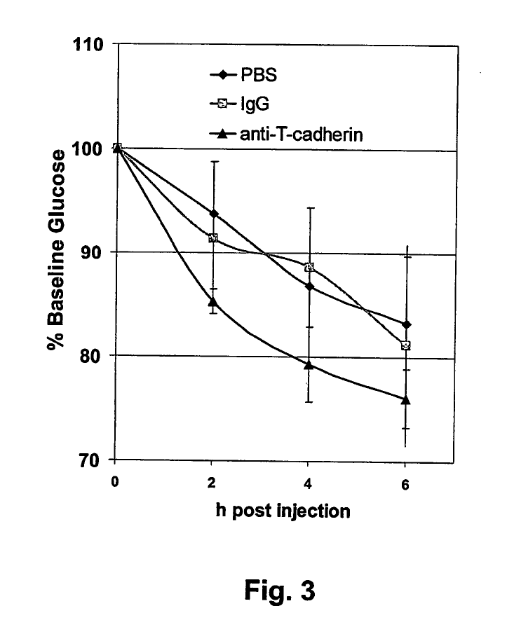

To test the potential role of adiponectin T-cadherin interaction in the context of blood glucose level, a T-cadherin specific antibody was tested for its ability to influence blood glucose levels. A model described previously to show blood glucose lowering effects with adiponectin was chosen (Pajvani U. P. et al, J. Biol. Chem. 278, March 2003: 9073-9085). Briefly 12-15 week old male FVB mice were used for the experiments. After two hours of food withdrawal, the mice were injected intravenously with 300 μl of PBS, 100 μg of rabbit gamma globulin (Jackson ImmunoResearch, No. 011-000-002) in 300 μl PBS or 100 μg of anti-T-cadherin antibody (Santa Cruz, No. sc-7940) in 300 μl PBS. The blood glucose levels were monitored immediately before the injection and at different time points after the injection (2 h, 4 h and 6 h) using a trinder assay. During the whole course of the experiments, the animals were not given access to food. Since the different animals vary in their individual glucose levels, all the values are expressed as percent of baseline glucose level. The baseline glucose levels are defined as the glucose value immediately before the injections. The results are shown in FIG. 3. As expected from the prolonged starvation period, blood glucose levels droop during the course of the experiment in all three groups. However a clearly faster decline was observed in the animals injected with the antibody against T-cadherin as compared to the PBS injected or rabbit gamma globulin injected control animals. These results demonstrate that an antibody against T-cadherin mimics the action of adiponectin in respect to blood glucose lowering.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1. A method for determining whether a test agent mimics an action of adiponectin, said method comprising:
(a) obtaining a test agent that interacts with T-cadherin;
(b) administering said test agent to a first animal;
(c) measuring one or more physiological parameters in said first animal after administration of said test agent;
(d) comparing said one or more physiological parameters in said first animal after administration of said test agent to said one or more physiological parameters in one or more control subjects, wherein said one or more control subjects are selected from the group consisting of:
i. said first animal prior to the administration of said test agent; and
i. a second animal to which said test agent has not been administered;
wherein said one or more physiological parameters are selected from the group consisting of blood glucose concentration, blood free fatty acid concentration, blood triglyceride concentration, glucose production in the presence of sub-physiological concentrations of insulin, neointimal thickening, lesions associated with coronary artery disease, and body weight; and
wherein a reduction of said one or more physiological parameters measured in said first animal after the administration of said test agent, as compared to said one or more physiological parameters in said one or more control subjects, identifies said test agent as mimicking an action of adiponectin.

2. The method of claim 1, wherein said test agent is an antibody.

3. The method of claim 2, wherein said antibody is a monoclonal antibody.

4. (canceled)

5. (canceled)

6. (canceled)

7. The method of claim 1, wherein said first and second animals are genetically modified mice.

8. The method of claim 7, wherein said genetically modified mice are selected from the group consisting of ob/ob mice, NOD mice and ApoE-deficient mice.

9. (canceled)

10. A method for determining whether a test agent mimics an action of adiponectin, said method comprising:
(a) obtaining a test agent that interacts with T-cadherin;
(b) contacting a first cell with said test agent, wherein said first cell expresses T-cadherin;
(c) measuring one or more cellular parameters in said first cell after contacting said first cell with said test agent;
(d) comparing said one or more cellular parameters in said first cell after contacting said first cell with said test agent to said one or more cellular parameters in one or more control cells, wherein said one or more control cells are selected from the group consisting of:
i. said first cell prior to contacting said first cell with said test agent;
ii. a second cell that has not been in contact with said test agent; and
iii. a second cell that does not express T-cadherin;
wherein said one or more cellular parameters are selected from the group consisting of:
(i) fatty acid oxidation, glucose uptake, insulin sensitivity, lactate production, 5′-AMP-activated protein kinase (AMPK) phosphorylation, acetyl coenzyme A carboxylase (ACC) phosphorylation, and IRS-1-mediated PI-3-kinase activity; and
wherein an increase in said one or more cellular parameters measured in said first cell after contacting said first cell with said test agent, as compared to said cellular parameter in said one or more control cells, identifies said test agent as mimicking an action of adiponectin or
(ii) smooth muscle cell proliferation, smooth muscle cell migration, TNF-α-induced NF-κB signaling, and TNF-α-induced expression of adhesion molecules; and wherein a decrease in said one or more cellular parameters measured in said first cell after contacting said first cell with said test agent, as compared to said one or more cellular parameters in said one or more control cells, identifies said test agent as mimicking an action of adiponectin.

11. The method of claim 10, wherein said test agent is an antibody.

12. The method of claim 11, wherein said antibody is a monoclonal antibody.

13. The method of claim 10, wherein said first and second cells are animal cells.

14. (canceled)

15. The method of claim 10, wherein said first and second cells are selected from the group consisting of C2C12 cells, human aortic smooth muscle cells (HASMCs), and human aortic endothelial cells (HAECs).

16. (canceled)

17. (canceled)

18. (canceled)

19. (canceled)

20. (canceled)

21. (canceled)

22. A method for determining whether a test agent mimics an action of adiponectin, said method comprising:
(a) contacting a first cell with a test agent, wherein said first cell expresses T-cadherin;
(b) contacting a second cell with said test agent, wherein said second cell does not express T-cadherin;
(c) measuring one or more cellular parameters in said first and second cells after contacting said first and second cells with said test agent;
(d) comparing said one or more cellular parameters in said first cell to said one or more cellular parameters in said second cell after contacting said first and second cells with test agent;
wherein said one or more cellular parameters are selected from the group consisting of:
(i) fatty acid oxidation, glucose uptake, insulin sensitivity, lactate production, 5′-AMP-activated protein kinase (AMPK) phosphorylation, acetyl coenzyme A carboxylase (ACC) phosphorylation, and IRS-1-mediated PI-3-kinase activity; and wherein an increase in said one or more cellular parameters measured in said first cell as compared to said one or more cellular parameters measured in said second cell, after contacting said first and second cells with said test agent, identifies said test agent as mimicking an action of adiponectin or
(ii) smooth muscle cell proliferation, smooth muscle cell migration, TNF-α-induced NF-κB signaling, and TNF-α-induced expression of adhesion molecules; and wherein a decrease in said one or more cellular parameters measured in said first cell as compared to said one or more cellular parameters measured in said second cell, after contacting said first and second cells with said test agent, identifies said test agent as mimicking an action of adiponectin.

23. The method of claim 22, wherein said test agent is an antibody.

24. The method of claim 23, wherein said antibody is an antibody that interacts with T-cadherin.

25. The method of claim 23, wherein said antibody is a monoclonal antibody.

26. (canceled)

27. (canceled)

28. (canceled)

29. (canceled)

30. (canceled)

31. A method for determining whether a test agent inhibits or enhances the interaction between adiponectin and T-cadherin, said method comprising:
(a) providing a test mixture comprising: (i) adiponectin or a fragment or derivative thereof; (ii) T-cadherin or a fragment or derivative thereof, one of them being fused to a detectable marker or enzyme and the other being immobilized on a suitable carrier; or (ii) T-cadherin or a fragment thereof, one of them being fused to a detectable marker or enzyme and the other being expressed on the surface of a cell; and (iii) a test agent;
(b) providing a control mixture comprising: (i) adiponectin or a fragment or derivative thereof; and (ii) T-cadherin or a fragment or derivative thereof, one of them being fused to a detectable marker or enzyme and the other being immobilized on a suitable carrier; or (ii) T-cadherin or a fragment thereof, one of them being fused to a detectable marker or enzyme and the other being expressed on the surface of a cell;
(c) removing unbound adiponectin and T-cadherin from said test mixture and from said control mixture; and
(d) measuring the signal produced by said marker or enzyme in said test mixture and in said control mixture;
wherein a decrease in the signal produced by said marker or enzyme in said test mixture as compared to the signal produced by said marker or enzyme in said control mixture indicates the ability of said test agent to inhibit the interaction between adiponectin and T-cadherin; and
wherein an increase in the signal produced by said marker or enzyme in said test mixture as compared to the signal produced by said marker or enzyme in said control mixture indicates the ability of said test agent to enhance the interaction between adiponectin and T-cadherin.

32. The method of claim 31, wherein said test agent is an antibody.

33. The method of claim 32, wherein said antibody is an antibody that interacts with T-cadherin.

34. The method of claim 32, wherein said antibody is a monoclonal antibody.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A method for treating and/or preventing a disease or condition in a mammal related to a deficiency or over-abundance of adiponectin, wherein a disease or condition related to a deficiency or over-abundance of adiponectin is a disease or condition selected from the group consisting of hypoadiponectinemia, obesity, anorexia nervosa, coronary artery disease, type I diabetes, type II diabetes, and atherosclerosis, said method comprising administering to the mammal a therapeutically effective amount of an agent or substance or monoclonal antibody that mimics an action of adiponectin, or that inhibits or enhances the interaction between adiponectin and T-cadherin.

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. Use of T-Cadherin, or a fragment or derivative thereof, for the preparation of a medicament for the treatment and/or prevention of a disease or condition related to a deficiency or over-abundance of adiponectin, wherein said disease or condition is selected from the group consisting of hypoadiponectinemia, obesity, anorexia nervosa, coronary artery disease, type I diabetes, type II diabetes, and atherosclerosis.

69. (canceled)

70. (canceled)

71. (canceled)

72. A monoclonal antibody that mimics an action of adiponectin, or that inhibits or enhances the interaction between adiponectin and T-cadherin for use as a medicament for the treatment and/or prevention of a disease or condition related to a deficiency or over-abundance of adiponectin, wherein said disease or condition is selected from the group consisting of hypoadiponectinemia, obesity, anorexia nervosa, coronary artery disease, type I diabetes, type II diabetes, and atherosclerosis.

73. (canceled)

74. (canceled)

75. (canceled)

76. (canceled)

77. (canceled)

78. (canceled)

79. (canceled)

80. (canceled)

81. (canceled)

82. (canceled)

83. (canceled)


SEQUENCE LISTING (59 sequences)

SEQ ID NO: 1 - DNA, 31 bp, artificial sequence
  primer
gcggatccagaagatgacgttactacaactg

SEQ ID NO: 2 - DNA, 32 bp, artificial sequence
  primer
gcctctagagagttggtatcatggtagagaag

SEQ ID NO: 3 - DNA, 30 bp, artificial sequence
  primer
gcggatccaaaaggagagcctggagaagcc

SEQ ID NO: 4 - protein, 230 aa, mus musculus
EDDVTTTEELAPALVPPPKGTCAGWMAGIPGHPGHNGTPGRDGRDGTPGEKGEKGDAGLLGPKGETGDVGVTGAEGPRGFPGTPGRKGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 5 - protein, 144 aa, mus musculus
KGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 6 - DNA, 1276 bp, mus musculus
  CDS (46)..(786)
ctctaaagattgtcagtggatctgacgacaccaaaagggctcaggatgctactgttgcaagctctcctgttcctcttaatcctgcccagtcatgccgaagatgacgttactacaactgaagagctagctcctgctttggtccctccacccaagggaacttgtgcaggttggatggcaggcatcccaggacatcctggccacaatggcacaccaggccgtgatggcagagatggcactcctggagagaagggagagaaaggagatgcaggtcttcttggtcctaagggtgagacaggagatgttggaatgacaggagctgaagggccacggggcttccccggaacccctggcaggaaaggagagcctggagaagccgcttatatgtatcgctcagcgttcagtgtggggctggagacccgcgtcactgttcccaatgtacccattcgctttactaagatcttctacaaccaacagaatcattatgacggcagcactggcaagttctactgcaacattccgggactctactacttctcttaccacatcacggtgtacatgaaagatgtgaaggtgagcctcttcaagaaggacaaggccgttctcttcacctacgaccagtatcaggaaaagaatgtggaccaggcctctggctctgtgctcctccatctggaggtgggagaccaagtctggctccaggtgtatggggatggggaccacaatggactctatgcagataacgtcaacgactctacatttactggctttcttctctaccatgataccaactgactgcaactacccatagcccatacaccaggagaatcatggaacagtcgacacactttcagcttagtttgagagattgattttattgcttagtttgagagtcctgagtattatccacacgtgtactcacttgttcattaaacgactttataaaaaataatttgtgttcctagtccagaaaaaaaggcactccctggtctccacgactcttacatggtagcaataacagaatgaaaatcacatttggtatgggggcttcacaatattcgcatgactgtctggaagtagaccatgctatttttctgctcactgtacacaaatattgttcacataaaccctataatgtaaatatgaaatacagtgattactcttctcacaggctgagtgtatgaatgtctaaagacccataagtattaaagtggtagggataaattggaaaaaaaaaaaaaaaaaaagaaaaactttagagcacactggcggccgttactag

SEQ ID NO: 7 - protein, 247 aa, mus musculus
MLLLQALLFLLILPSHAEDDVTTTEELAPALVPPPKGTCAGWMAGIPGHPGHNGTPGRDGRDGTPGEKGEKGDAGLLGPKGETGDVGMTGAEGPRGFPGTPGRKGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTN

SEQ ID NO: 8 - protein, 265 aa, mus musculus
METDTLLLWVLLLWVPGSTGDADPEDDVTTTEELAPALVPPPKGTCAGWMAGIPGHPGHNGTPGRDGRDGTPGEKGEKGDAGLLGPKGETGDVGVTGAEGPRGFPGTPGRKGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTNSLDDYKDDDDK

SEQ ID NO: 9 - protein, 183 aa, mus musculus
METDTLLLWVLLLWVPGSTGDADPKGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTNSLAGGSGDYKDDDDK

SEQ ID NO: 10 - protein, 194 aa, mus musculus
METDTLLLWVLLLWVPGSTGDAAQPARRADYKDDDDKPGGTKLADPKGEPGEAAYMYRSAFSVGLETRVTVPNVPIRFTKIFYNQQNHYDGSTGKFYCNIPGLYYFSYHITVYMKDVKVSLFKKDKAVLFTYDQYQEKNVDQASGSVLLHLEVGDQVWLQVYGDGDHNGLYADNVNDSTFTGFLLYHDTNSLDN

SEQ ID NO: 11 - DNA, 68 bp, artificial sequence
  primer
  misc_feature (68)..(68): n is a, c, g, or t
aagcagtggtatcaacgcagagtggccgaggcggccttttttttttttttttttttttttttttttvn

SEQ ID NO: 12 - DNA, 36 bp, artificial sequence
  primer
aagcagtggtatcaacgcagagtggccattacggcc

SEQ ID NO: 13 - DNA, 23 bp, artificial sequence
  primer
aagcagtggtatcaacgcagagt

SEQ ID NO: 14 - DNA, 22 bp, artificial sequence
  primer
acaaattggactaatcgatggc

SEQ ID NO: 15 - DNA, 50 bp, artificial sequence
  primer
ggggacaagtttgtacaaaaaagcaggctctacaacaccaccacctctag

SEQ ID NO: 16 - DNA, 51 bp, artificial sequence
  primer
ggggaccactttgtacaagaaagctgggtacaaattggactaatcgatggc

SEQ ID NO: 17 - DNA, 22 bp, artificial sequence
  primer
atacgactcactatagggagac

SEQ ID NO: 18 - DNA, 20 bp, artificial sequence
  primer
cagccgagaactccgctcac

SEQ ID NO: 19 - DNA, 20 bp, artificial sequence
  primer
cggtgagccggaacttggac

SEQ ID NO: 20 - protein, 244 aa, homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 21 - DNA, 3823 bp, homo sapiens
  CDS (97)..(2235)
agcggctctcctcaaagcctggctcccacggaaaatatgctcagtgcagccgcgtgcatgaatgaaaacgccgccgggcgcttctagtcggacaaaatgcagccgagaactccgctcgttctgtgcgttctcctgtcccaggtgctgctgctaacatctgcagaagatttggactgcactcctggatttcagcagaaagtgttccatatcaatcagccagctgaattcattgaggaccagtcaattctaaacttgaccttcagtgactgtaagggaaacgacaagctacgctatgaggtctcgagcccatacttcaaggtgaacagcgatggcggcttagttgctctgagaaacataactgcagtgggcaaaactctgttcgtccatgcacggaccccccatgcggaagatatggcagaactcgtgattgtcggggggaaagacatccagggctccttgcaggatatatttaaatttgcaagaacttctcctgtcccaagacaaaagaggtccattgtggtatctcccattttaattccagagaatcagagacagcctttcccaagagatgttggcaaggtagtcgatagtgacaggccagaaaggtccaagttccggctcactggaaagggagtggatcaagagcctaaaggaattttcagaatcaatgagaacacagggagcgtctccatgacacggaccttggacagagaagtaatcgctgtttatcaactatttgtggagaccactgatgtcaatggcaaaactctcgaggggccggtgcctctggaagtcattgtgattgatcagaatgacaaccgaccgatctttcgggaaggcccctacatcggccacgtcatggaagggtcacccacaggcaccacagtgatgcggatgacagcctttgatgcagatgacccagccaccgataatgccctcctgcggtataatatccgtcggcagacgcctgacaagccatctcccaacatgttctacatcgatcctgagaaaggagacattgtcactgttgtgtcacctgcgctgctggaccgagagactctggaaaatcccaagtatgaactgatcatcgaggctcaagatatggctggactggatgttggattaacaggcacggccacagccacgatcatgatcgatgacaaaaatgatcactcaccaaaattcaccaagaaagagtttcaagccacagtcgaggaaggagctgtgggagttattgtcaatttgacagttgaagataaggatgaccccgccacaggtgcatggagggctgcctacaccatcatcaacggaaaccccgggcagagctttgaaatccacaccaaccctcaaaccaacgaagggatgctttctgttgtcaaaccattggactatgaaatttctgccttccacaccctgctgatcaaagtggaaaatgaagacccactcgtacccgacgtctcctacggccccagctccacagccaccgtccacatcactgtcctggatgtcaacgagggcccagtcttctacccagaccccatgatggtgaccaggcaggaggacctctctgtgggcagcgtgctgctgacagtgaatgccacggaccccgactccctgcagcatcaaaccatcaggtattctgtttacaaggacccagcaggttggctgaatattaaccccatcaatgggactgttgacaccacagctgtgctggaccgtgagtccacatttgtcgacaacagcgtgtacactgctctcttcctggcaattgacagtggcaaccctcccgctacgggcactgggactttgctgataaccctggaggacgtgaatgacaatgccccgttcatttaccccacagtagctgaagtctgtgatgatgccaaaaacctcagtgtagtcattttgggagcatcagataaggatcttcacccgaatacagatcctttcaaatttgaaatccacaaacaagctgttcctgataaagtctggaagatctccaagatcaacaatacacacgccctggtaagccttcttcaaaatctgaacaaagcaaactacaacctgcccatcatggtgacagattcagggaaaccacccatgacgaatatcacagatctcagggtacaagtgtgctcctgcaggaattccaaagtggactgcaacgcggcgggggccctgcgcttcagcctgccctcagtcctgctcctcagcctcttcagcttagcttgtctgtgagaactcctgacgtctgaagcttgactcccaagtttccatagcaacaggaaaaaaaaaaatctatccaaatctgaagattgcggtttacagctatcgaacttcacaactaggcctcaattgttccggttttttattttctttacaatttcacttagtctgtacttcatcattttgacagcatcttcctccctcctttaattaatggaatcttctgaattttccctgaatgtttaaagatcatgacatatgacttgatcttctgggagcaggaacaatgactactttttctggtgtgttaacatgtcgctagccagtgctccaggcacccagctttgtctgtgggttagtattggtgtatgtatgagtatctgtatgtatatatacacggtatttatagagagagactatcctggagaagcctcgttttgatgccattcttccttgcaaggttaagcaaggtgggtggaaactaagacacctgaaccctccagggcctcccgcatcaaggtcagcatgaggacagaccacagagctgtcacttttgctccgaagctacttctccactgtcccgttcagtctgaatgctgccacaaccagccaggcaggtccacagagagggagagcagagaaagaagtcctttctctttattgagttcgaggactacaaccaatttacactgccatctgatgccgtgatcctgagccaaggaggtgaggagcagagcaggcaatttcaccaccaaatgccaagaaaagggctgacattttctttcatgggcaccaacctgcatttgtatgtgtcccgaatccacagtcgtactgattctaatggggacacagatcatggtagagaatctctccctcctcagtaaatgtacaactgcacctgtcatcatggaggtcatacatgcatacaaagaggtgtacaggtaccatcttgtatacacatatatacccacatgtacagacatacatttatgcgcattcacgctgtttgtttcatatatacaggcataaaatagagtaaatacaggtagttttaaaagtacccttttgtgtgaattgactaccgttgtttgcaaacccgaaaataaaagacgttcattatgtatgaaaagtaactgatttgtattctgtgagcatgtaaaagcggaaagttagtgcttgttttaagattaccttcttgttgataaaccataaatgaatcatcaaagctcacaccaaatttttctatcaaataaaactagtgacagcttgtggctttttattagagctcgccacgaactagggtaaggtgagtgtcttagcatattttaatgcagttgcttactaaaggttttaaccgcacatgcacacacacacgctttcttatgcaatctatgtttgcacttgtgctttcagttagccttctgtaggaagtagaagtcatatgttgtctttgttgtagtgaaattatacagatagagttccatatattgtatttgtttcaatggtaaatccttttggaacatatagaatgcagagatttttttttccattaaaataaatgggtattggtggttaaaactgaaaaaaaaaaaaaaaa

SEQ ID NO: 22 - protein, 713 aa, homo sapiens
MQPRTPLVLCVLLSQVLLLTSAEDLDCTPGFQQKVFHINQPAEFIEDQSILNLTFSDCKGNDKLRYEVSSPYFKVNSDGGLVALRNITAVGKTLFVHARTPHAEDMAELVIVGGKDIQGSLQDIFKFARTSPVPRQKRSIVVSPILIPENQRQPFPRDVGKVVDSDRPERSKFRLTGKGVDQEPKGIFRINENTGSVSMTRTLDREVIAVYQLFVETTDVNGKTLEGPVPLEVIVIDQNDNRPIFREGPYIGHVMEGSPTGTTVMRMTAFDADDPATDNALLRYNIRRQTPDKPSPNMFYIDPEKGDIVTVVSPALLDRETLENPKYELIIEAQDMAGLDVGLTGTATATIMIDDKNDHSPKFTKKEFQATVEEGAVGVIVNLTVEDKDDPATGAWRAAYTIINGNPGQSFEIHTNPQTNEGMLSVVKPLDYEISAFHTLLIKVENEDPLVPDVSYGPSSTATVHITVLDVNEGPVFYPDPMMVTRQEDLSVGSVLLTVNATDPDSLQHQTIRYSVYKDPAGWLNINPINGTVDTTAVLDRESTFVDNSVYTALFLAIDSGNPPATGTGTLLITLEDVNDNAPFIYPTVAEVCDDAKNLSVVILGASDKDLHPNTDPFKFEIHKQAVPDKVWKISKINNTHALVSLLQNLNKANYNLPIMVTDSGKPPMTNITDLRVQVCSCRNSKVDCNAAGALRFSLPSVLLLSLFSLACL

SEQ ID NO: 23 - DNA, 3823 bp, homo sapiens; identical to SEQ ID NO: 21
  CDS (97)..(2235)

SEQ ID NO: 24 - protein, 713 aa, homo sapiens; identical to SEQ ID NO: 22

SEQ ID NO: 25 - DNA, 2690 bp, homo sapiens
  CDS (445)..(2583)
cttcaaggttttgctgactcagtctggtagtcagagtctgcaggagaagacagttcaaggcagggcctggaggattggatcagtttagggacaggtcaaaggctggcttagagaccttagaggcaggttgcttgggtcgttgaatgctagtctggtcctgagagcccttttctctggcaactgtggactcagagctaaccaattgtagttggcagtgggggtgaagggtgatccagaggcctgagctgcagagggcacaagagagaaaagatgtcttagaaagagctttgagaacatgccttggctgctggcagggaccttggatggggtagtctacacccggaagtgcctgcctgccatcctctagtggctgccttgcaaaatatgctcagtgcagccgcgtgcatgaatgaaaacgccgccgggcgcttctagtcggacaaaatgcagccgagaactccgctcgttctgtgcgttctcctgtcccaggtgctgctgctaacatctgcagaagatttggactgcactcctggatttcagcagaaagtgttccatatcaatcagccagctgaattcattgaggaccagtcaattctaaacttgaccttcagtgactgtaagggaaacgacaagctacgctatgaggtctcgagcccatacttcaaggtgaacagcgatggcggcttagttgctctgagaaacataactgcagtgggcaaaactctgttcgtccatgcacggaccccccatgcggaagatatggcagaactcgtgattgtcggggggaaagacatccagggctccttgcaggatatatttaaatttgcaagaacttctcctgtcccaagacaaaagaggtccattgtggtatctcccattttaattccagagaatcagagacagcctttcccaagagatgttggcaaggtagtcgatagtgacaggccagaaaggtccaagttccggctcactggaaagggagtggatcaagagcctaaaggaattttcagaatcaatgagaacacagggagcgtctccgtgacacggaccttggacagagaagtaatcgctgtttatcaactatttgtggagaccactgatgtcaatggcaaaactctcgaggggccggtgcctctggaagtcattgtgattgatcagaatgacaaccgaccgatctttcgggaaggcccctacatcggccacgtcatggaagggtcacccacaggcaccacagtgatgcggatgacagcctttgatgcagatgacccagccaccgataatgccctcctgcggtataatatccgtcaacagacgcctgacaagccatctcccaacatgttctacatcgatcctgagaaaggagacattgtcactgttgtgtcacctgcgctgctggaccgagagactctggaaaatcccaagtatgaactgatcatcgaggctcaagatatggctggactggatgttggattaacaggcacggccacagccacgatcatgatcgatgacaaaaatgatcactcaccaaaattcaccaagaaagagtttcaagccacagtcgaggaaggagctgtgggagttattgtcaatttgacagttgaagataaggatgaccccaccacaggtgcatggagggctgcctacaccatcatcaacggaaaccccgggcagagctttgaaatccacaccaaccctcaaaccaacgaagggatgctttctgttgtcaaaccattggactatgaaatttctgccttccacaccctgctgatcaaagtggaaaatgaagacccactcgtacccgacgtctcctacggccccagctccacagccaccgtccacatcactgtcctggatgtcaacgagggcccagtcttctacccagaccccatgatggtgaccaggcaggaggacctctctgtgggcagcgtgctgctgacagtgaatgccacggaccccgactccctgcagcatcaaaccatcaggtattctgtttacaaggacccagcaggttggctgaatattaaccccatcaatgggactgttgacaccacagctgtgctggaccgtgagtccccatttgtcgacaacagcgtgtacactgctctcttcctggcaattgacagtggcaaccctcccgctacgggcactgggactttgctgataaccctggaggacgtgaatgacaatgccccgttcatttaccccacagtagctgaagtctgtgatgatgccaaaaacctcagtgtagtcattttgggagcatcagataaggatcttcacccgaatacagatcctttcaaatttgaaatccacaaacaagctgttcctgataaagtctggaagatctccaagatcaacaatacacacgccctggtaagccttcttcaaaatctgaacaaagcaaactacaacctgcccatcatggtgacagattcagggaaaccacccatgacgaatatcacagatctcagggtacaagtgtgctcctgcaggaattccaaagtggactgcaacgcggcgggggccctgcgcttcagcctgccctcagtcctgctcctcagcctcttcagcttagcttgtctgtgagaactcctgacgtctgaagcttgactcccaagtttccatagcaacaggaaaaaaaaaaaatctatccaaatctgaagattgcggtttacagctatcgaacttcg

SEQ ID NO: 26 - protein, 713 aa, homo sapiens
MQPRTPLVLCVLLSQVLLLTSAEDLDCTPGFQQKVFHINQPAEFIEDQSILNLTFSDCKGNDKLRYEVSSPYFKVNSDGGLVALRNITAVGKTLFVHARTPHAEDMAELVIVGGKDIQGSLQDIFKFARTSPVPRQKRSIVVSPILIPENQRQPFPRDVGKVVDSDRPERSKFRLTGKGVDQEPKGIFRINENTGSVSVTRTLDREVIAVYQLFVETTDVNGKTLEGPVPLEVIVIDQNDNRPIFREGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIMIDDKNDHSPKFTKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTRQEDLSVGSVLLTVNATDPDSLQHQTIRYSVYKDPAGWLNINPINGTVDTTAVLDRESPFVDNSVYTALFLAIDSGNPPATGTGTLLITLEDVNDNAPFIYPTVAEVCDDAKNLSVVILGASDKDLHPNTDPFKFEIHKQAVPDKVWKISKINNTHALVSLLQNLNKANYNLPIMVTDSGKPPMTNITDLRVQVCSCRNSKVDCNAAGALRFSLPSVLLLSLFSLACL

SEQ ID NO: 27 - DNA, 3840 bp, homo sapiens
  CDS (121)..(2259)
gggaagttggctggctggcgaggcagagcctctcctcaaagcctggctcccacggaaaatatgctcagtgcagccgcgtgcatgaatgaaaacgccgccgggcgcttctagtcggacaaaatgcagccgagaactccgctcgttctgtgcgttctcctgtcccaggtgctgctgctaacatctgcagaagatttggactgcactcctggatttcagcagaaagtgttccatatcaatcagccagctgaattcattgaggaccagtcaattctaaacttgaccttcagtgactgtaagggaaacgacaagctacgctatgaggtctcgagcccatacttcaaggtgaacagcgatggcggcttagttgctctgagaaacataactgcagtgggcaaaactctgttcgtccatgcacggaccccccatgcggaagatatggcagaactcgtgattgtcggggggaaagacatccagggctccttgcaggatatatttaaatttgcaagaacttctcctgtcccaagacaaaagaggtccattgtggtatctcccattttaattccagagaatcagagacagcctttcccaagagatgttggcaaggtagtcgatagtgacaggccagaaaggtccaagttccggctcactggaaagggagtggatcaagagcctaaaggaattttcagaatcaatgagaacacagggagcgtctccgtgacacggaccttggacagagaagtaatcgctgtttatcaactatttgtggagaccactgatgtcaatggcaaaactctcgaggggccggtgcctctggaagtcattgtgattgatcagaatgacaaccgaccgatctttcgggaaggcccctacatcggccacgtcatggaagggtcacccacaggcaccacagtgatgcggatgacagcctttgatgcagatgacccagccaccgataatgccctcctgcggtataatatccgtcagcagacgcctgacaagccatctcccaacatgttctacatcgatcctgagaaaggagacattgtcactgttgtgtcacctgcgctgctggaccgagagactctggaaaatcccaagtatgaactgatcatcgaggctcaagatatggctggactggatgttggattaacaggcacggccacagccacgatcatgatcgatgacaaaaatgatcactcaccaaaattcaccaagaaagagtttcaagccacagtcgaggaaggagctgtgggagttattgtcaatttgacagttgaagataaggatgaccccaccacaggtgcatggagggctgcctacaccatcatcaacggaaaccccgggcagagctttgaaatccacaccaaccctcaaaccaacgaagggatgctttctgttgtcaaaccattggactatgaaatttctgccttccacaccctgctgatcaaagtggaaaatgaagacccactcgtacccgacgtctcctacggccccagctccacagccaccgtccacatcactgtcctggatgtcaacgagggcccagtcttctacccagaccccatgatggtgaccaggcaggaggacctctctgtgggcagcgtgctgctgacagtgaatgccacggaccccgactccctgcagcatcaaaccatcaggtattctgtttacaaggacccagcaggttggctgaatattaaccccatcaatgggactgttgacaccacagctgtgctggaccgtgagtccccatttgtcgacaacagcgtgtacactgctctcttcctggcaattgacagtggcaaccctcccgctacgggcactgggactttgctgataaccctggaggacgtgaatgacaatgccccgttcatttaccccacagtagctgaagtctgtgatgatgccaaaaacctcagtgtagtcattttgggagcatcagataaggatcttcacccgaatacagatcctttcaaatttgaaatccacaaacaagctgttcctgataaagtctggaagatctccaagatcaacaatacacacgccctggtaagccttcttcaaaatctgaacaaagcaaactacaacctgcccatcatggtgacagattcagggaaaccacccatgacgaatatcacagatctcagggtacaagtgtgctcctgcaggaattccaaagtggactgcaacgcggcaggggccctgcgcttcagcctgccctcagtcctgctcctcagcctcttcagcttagcttgtctgtgagaactcctgacgtctgaagcttgactcccaagtttccatagcaacaggaaaaaaaaaaatctatccaatctgaagattgcggtttacagctatcgaacttcacaactaggcctcaattgttccggttttttattttctttacaatttcacttagtctgtacttcatcattttgacagcatcttcctccctcctttaattaatggaatcttctgaattttccctgaatgtttaaagatcatgacatatgacttgatcttctgggagcaggaacaatgactactttttctggtgtgttaacatgtcgctagccagtgctccaggcacccagctttgtctgtgggttagtattggtgtatgtatgagtatctgtatgtatatatacacggtatttatagagagagactatcctggagaagcctcgttttgatgccattcttccttgcaaggttaagcaaggtgggtggaaactaagacacctgaaccctccagggcctcccgcatcaaggtcagcatgaggacagaccacagagctgtcacttttgctccgaagctacttctccactgtcccgttcagtctgaatgctgccacaaccagccaggcaggtccacagagagggagagcagagaaagaagtcctttctctttattgagttcgaggactacaaccaatttacactgccatctgatgccgtgatcctgagccaaggaggtgaggagcagagcaggcaatttcaccaccaaatgccaagaaaagggctgacattttctttcatgggcaccaacctgcatttgtatgtgtcccgaatccacagtcgtactgattctaatggggacacagatcatggtagagaatctctccctcctcagtaaatgtacaactgcacctgtcatcatggaggtcatacatgcatacaaagaggtgtacaggtaccatcttgtatacacatatatacccacatgtacagacatacatttatgcacattcacgctgtttgtttcatatatacaggcataaaatagagtaaatacaggtagttttaaaagtacccttttgtgtgaattgactaccgttgtttgcaaacccgaaaataaaagacgttcattatgtatgaaaagtaactgatttgtattctgtgagcatgtaaaagcggaaagttagtgcttgttctaagattaccttcttgttgataaaccataaatgaatcatcaaagctcacaccaaatttttctatcaaataaaactagtgacagcttgtggctttttattagagctcgccacgaactagggtaaggtgagtgtcttagcatattttaatgcagttgcttactaaaggttttaaccgcacatgcacacacacacgctttcttatgcaatctatgtttgcacttgtgctttcagttagccttctgtaggaagtagaagtcatatgttgtctttgttgtagtgaaattatacagatagagttccatatattgtatttgtttcaatggtaaatccttttggaacatatagaatgcagagatttttttttccattaaaataaatgggtattggtggttaaaaaaaaaaaaaaaaa

SEQ ID NO: 28 - protein, 713 aa, homo sapiens; identical to SEQ ID NO: 26

SEQ ID NO: 29 - DNA, 3485 bp, homo sapiens
  CDS (217)..(2355)
tgatccagaggcctgagctgcagagggcacaagagagaaaagatgtcttagaaagagctttgagaacatgccttggctgctggcagggaccttggatggggtagtctacacccggaagtgcctgcctgccatcctctagtggctgccttgcaaaatatgctcagtgcagccgcgtgcatgaatgaaaacgccgccgggcgcttctagtcggacaaaatgcagccgagaactccgctcgttctgtgcgttctcctgtcccaggtgctgctgctaacatctgcagaagatttggactgcactcctggatttcagcagaaagtgttccatatcaatcagccagctgaattcattgaggaccagtcaattctaaacttgaccttcagtgactgtaagggaaacgacaagctacgctatgaggtctcgagcccatacttcaaggtgaacagcgatggcggcttagttgctctgagaaacataactgcagtgggcaaaactctgttcgtccatgcacggaccccccatgcggaagatatggcagaactcgtgattgtcggggggaaagacatccagggctccttgcaggatatatttaaatttgcaagaacttctcctgtcccaagacaaaagaggtccattgtggtatctcccattttaattccagagaatcagagacagcctttcccaagagatgttggcaaggtagtcgatagtgacaggccagaaaggtccaagttccggctcactggaaagggagtggatcaagagcctaaaggaattttcagaatcaatgagaacacagggagcgtctccgtgacacggaccttggacagagaagtaatcgctgtttatcaactatttgtggagaccactgatgtcaatggcaaaactctcgaggggccggtgcctctggaagtcattgtgattgatcagaatgacaaccgaccgatctttcgggaaggcccctacatcggccacgtcatggaagggtcacccacaggcaccacagtgatgcggatgacagcctttgatgcagatgacccagccaccgataatgccctcctgcggtataatatccgtcaacagacgcctgacaagccatctcccaacatgttctacatcgatcctgagaaaggagacattgtcactgttgtgtcacctgcgctgctggaccgagagactctggaaaatcccaagtatgaactgatcatcgaggctcaagatatggctggactggatgttggattaacaggcacggccacagccacgatcatgatcgatgacaaaaatgatcactcaccaaaattcaccaagaaagagtttcaagccacagtcgaggaaggagctgtgggagttattgtcaatttgacagttgaagataaggatgaccccaccacaggtgcatggagggctgcctacaccatcatcaacggaaaccccgggcagagctttgaaatccacaccaaccctcaaaccaacgaagggatgctttctgttgtcaaaccattggactatgaaatttctgccttccacaccctgctgatcaaagtggaaaatgaagacccactcgtacccgacgtctcctacggccccagctccacagccaccgtccacatcactgtcctggatgtcaacgagggcccagtcttctacccagaccccatgatggtgaccaggcaggaggacctctctgtgggcagcgtgctgctgacagtgaatgccacggaccccgactccctgcagcatcaaaccatcaggtattctgtttacaaggacccagcaggttggctgaatattaaccccatcaatgggactgttgacaccacagctgtgctggaccgtgagtccccatttgtcgacaacagcgtgtacactgctctcttcctggcaattgacagtggcaaccctcccgctacgggcactgggactttgctgataaccctggaggacgtgaatgacaatgccccgttcatttaccccacagtagctgaagtctgtgatgatgccaaaaacctcagtgtagtcattttgggagcatcagataaggatcttcacccgaatacagatcctttcaaatttgaaatccacaaacaagctgttcctgataaagtctggaagatctccaagatcaacaatacacacgccctggtaagccttcttcaaaatctgaacaaagcaaactacaacctgcccatcatggtgacagattcagggaaaccacccatgacgaatatcacagatctcagggtacaagtgtgctcctgcaggaattccaaagtggactgcaacgcggcgggggccctgcgcttcagcctgccctcagtcctgctcctcagcctcttcagcttagcttgtctgtgagaactcctgacgtctgaagcttgactcccaagtttccatagcaacaggaaaaaaaaaaaatctatccaaatctgaagattgcggtttacagctatcgaacttcacaactaggcctcaattgttccggttttttattttctttacaatttcacttagtctgtacttcatcattttgacagcatcttcctccctcctttaattaatggaatcttctgaattttccctgaatgtttaaagatcatgacatatgacttgatcttctgggagcaggaacaatgactactttttctggtgtgttaacatgtcgctagccagtgctccaggcacccagctttgtctgtgggttagtattggtgtatgtatgagtatctgtatgtatatatacacggtatttatagagagagactatctggagaagcctcgttttgatgccattcttccttgcaaggttaagcaaggtgggtggaaactaagacacctgaaccctccagcctcccgcatcaaggtcagcatgaggacagaccacagagctgtcacttttgctccgaagctacttctccactgtcccgttcagtctgaatgtgccacaaccagccaggcaggtccacagagagggagagcagagaaagaagtcctttctctttattgagttcgaggactacaaccaatttacactgccatctgatgccgtgatcctgagccaaggaggtgaggagcagagcaggcaatttcaccaccaaatgccaagaaaagggctgacattttctttcatgggcaccaacctgcatttgtatgtgtcccgaatccacagtcgtactgattctaatggggacacagatcatggtagagaatctctccctcctcagtaaatgtacaactgcacctgttatcatggaggtcatacatggatacaaagaggtgtacaggtaccatcttgtatacacatatatacccacatgtacagacatacatttatgcacattcacgctgtttgtttcatatatacaggcataaaatagagtaaatacaggtagttttaaaagtacccttttgtgtgaattgactaccgttgtttgcaaacccgaaaataaaagacgttcattatgtatgaaaaaaaaaaaaaaaaa

SEQ ID NO: 30 - protein, 713 aa, homo sapiens; identical to SEQ ID NO: 26

SEQ ID NO: 31 - DNA, 3926 bp, homo sapiens
  CDS (217)..(2355)
tgatccagaggcctgagctgcagagggcacaagagagaaaagatgtcttagaaagagctttgagaacatgccttggctgctggcagggaccttggatggggtagtctacacccggaagtgcctgcctgccatcctctagtggctgccttgcaaaatatgctcagtgcagccgcgtgcatgaatgaaaacgccgccgggcgcttctagtcggacaaaatgcagccgagaactccgctcgttctgtgcgttctcctgtcccaggtgctgctgctaacatctgcagaagatttggactgcactcctggatttcagcagaaagtgttccatatcaatcagccagctgaattcattgaggaccagtcaattctaaacttgaccttcagtgactgtaagggaaacgacaagctacgctatgaggtctcgagcccatacttcaaggtgaacagcgatggcggcttagttgctctgagaaacataactgcagtgggcaaaactctgttcgtccatgcacggaccccccatgcggaagatatggcagaactcgtgattgtcggggggaaagacatccagggctccttgcaggatatatttaaatttgcaagaacttctcctgtcccaagacaaaagaggtccattgtggtatctcccattttaattccagagaatcagagacagcctttcccaagagatgttggcaaggtagtcgatagtgacaggccagaaaggtccaagttccggctcactggaaagggagtggatcaagagcctaaaggaattttcagaatcaatgagaacacagggagcgtctccgtgacacggaccttggacagagaagtaatcgctgtttatcaactatttgtggagaccactgatgtcaatggcaaaactctcgaggggccggtgcctctggaagtcattgtgattgatcagaatgacaaccgaccgatctttcgggaaggcccctacatcggccacgtcatggaagggtcacccacaggcaccacagtgatgcggatgacagcctttgatgcagatgacccagccaccgataatgccctcctgcggtataatatccgtcaacagacgcctgacaagccatctcccaacatgttctacatcgatcctgagaaaggagacattgtcactgttgtgtcacctgcgctgctggaccgagagactctggaaaatcccaagtatgaactgatcatcgaggctcaagatatggctggactggatgttggattaacaggcacggccacagccacgatcatgatcgatgacaaaaatgatcactcaccaaaattcaccaagaaagagtttcaagccacagtcgaggaaggagctgtgggagttattgtcaatttgacagttgaagataaggatgaccccaccacaggtgcatggagggctgcctacaccatcatcaacggaaaccccgggcagagctttgaaatccacaccaaccctcaaaccaacgaagggatgctttctgttgtcaaaccattggactatgaaatttctgccttccacaccctgctgatcaaagtggaaaatgaagacccactcgtacccgacgtctcctacggccccagctccacagccaccgtccacatcactgtcctggatgtcaacgagggcccagtcttctacccagaccccatgatggtgaccaggcaggaggacctctctgtgggcagcgtgctgctgacagtgaatgccacggaccccgactccctgcagcatcaaaccatcaggtattctgtttacaaggacccagcaggttggctgaatattaaccccatcaatgggactgttgacaccacagctgtgctggaccgtgagtccccatttgtcgacaacagcgtgtacactgctctcttcctggcaattgacagtggcaaccctcccgctacgggcactgggactttgctgataaccctggaggacgtgaatgacaatgccccgttcatttaccccacagtagctgaagtctgtgatgatgccaaaaacctcagtgtagtcattttgggagcatcagataaggatcttcacccgaatacagatcctttcaaatttgaaatccacaaacaagctgttcctgataaagtctggaagatctccaagatcaacaatacacacgccctggtaagccttcttcaaaatctgaacaaagcaaactacaacctgcccatcatggtgacagattcagggaaaccacccatgacgaatatcacagatctcagggtacaagtgtgctcctgcaggaattccaaagtggactgcaacgcggcgggggccctgcgcttcagcctgccctcagtcctgctcctcagcctcttcagcttagcttgtctgtgagaactcctgacgtctgaagcttgactcccaagtttccatagcaacaggaaaaaaaaaaaatctatccaaatctgaagattgcggtttacagctatcgaacttcacaactaggcctcaattgttccggttttttattttctttacaatttcacttagtctgtacttcatcattttgacagcatcttcctccctcctttaattaatggaatcttctgaattttccctgaatgtttaaagatcatgacatatgacttgatcttctgggagcaggaacaatgactactttttctggtgtgttaacatgtcgctagccagtgctccaggcacccagctttgtctgtgggttagtattggtgtatgtatgagtatctgtatgtatatatacacggtatttatagagagagactatctggagaagcctcgttttgatgccattcttccttgcaaggttaagcaaggtgggtggaaactaagacacctgaaccctccagcctcccgcatcaaggtcagcatgaggacagaccacagagctgtcacttttgctccgaagctacttctccactgtcccgttcagtctgaatgtgccacaaccagccaggcaggtccacagagagggagagcagagaaagaagtcctttctctttattgagttcgaggactacaaccaatttacactgccatctgatgccgtgatcctgagccaaggaggtgaggagcagagcaggcaatttcaccaccaaatgccaagaaaagggctgacattttctttcatgggcaccaacctgcatttgtatgtgtcccgaatccacagtcgtactgattctaatggggacacagatcatggtagagaatctctccctcctcagtaaatgtacaactgcacctgttatcatggaggtcatacatggatacaaagaggtgtacaggtaccatcttgtatacacatatatacccacatgtacagacatacatttatgcacattcacgctgtttgtttcatatatacaggcataaaatagagtaaatacaggtagttttaaaagtacccttttgtgtgaattgactaccgttgtttgcaaacccgaaaataaaagacgttcattatgacgaaaagtaactgatttgtattctgtggcatgtaaaagcggaaagttagtgcttgttctaagatgcctcctgttgataaaccataaatgaatcatcaaagctcacaccaaatttttctatcaaataaaactagtgacagcttgtggactttttattagagctcgccacgaactagggtaaggtgagtgtcttagcatattttaatgcagttgcttactaaaggttttaaccgcacacacacacgcatttcttatgcaatctatgtttcgacttgtgctttcagttagccttctgtaggaagtagaagtcatatgttgtctttgttgtagtgaaattatacagatagagttccatatattgtatttgtttcaatggtaaatccttttggaacatatagaatgcagagatttttttttccattaaaataaatgggtattggtggttaaaaaaaaaaaaaaaaa

SEQ ID NO: 32 - protein, 713 aa, homo sapiens; identical to SEQ ID NO: 26

SEQ ID NO: 33 - DNA, 1984 bp, homo sapiens
  misc_feature (9)..(9): n is a, c, g, or t
  misc_feature (17)..(18): n is a, c, g, or t
  misc_feature (33)..(33): n is a, c, g, or t
  misc_feature (41)..(41): n is a, c, g, or t
  misc_feature (49)..(50): n is a, c, g, or t
  misc_feature (52)..(52): n is a, c, g, or t
  misc_feature (54)..(54): n is a, c, g, or t
  misc_feature (62)..(62): n is a, c, g, or t
  misc_feature (80)..(80): n is a, c, g, or t
  misc_feature (84)..(84): n is a, c, g, or t
  misc_feature (127)..(127): n is a, c, g, or t
  misc_feature (157)..(157): n is a, c, g, or t
  misc_feature (159)..(159): n is a, c, g, or t
  misc_feature (175)..(175): n is a, c, g, or t
  misc_feature (207)..(209): n is a, c, g, or t
  misc_feature (214)..(214): n is a, c, g, or t
  misc_feature (1181)..(1181): n is a, c, g, or t
  exon (1669)..(1713)
agcttcagncatcactnncatctttatcaatgnccttgggnccacaggnngngnccttctgnccaaacagcagccaatgnaggnatcttaaaaggcacatcactttatatttattcagtggttatgncatcacttaatcctcaccacaaccttgtgngntcagtacaatttgtgntttcattttatctgtgaacaaatggaggcagnnntttgngtaatttacaagtaagtggcagatccgggggctgtgcctagacaatgtggctccagactccttgcccctccctgccttctgctgcatcttgattgagtctagccatctatttctttaacacacactaattgctgtctccatgttccaggccctgtgccagatgctagaaatacatccccatccactggatcttggcatggtccattgctcagtgtcctgctatgttacgtgttacataatgcatttattccggtatttagcccatggaaaataatgctgaaagacattgtatgcatgtttctacaacaaaactatgtaacttatgtttcttttattgctttggctatcaggaagctcttatccaaatcagagcaaatacattagaatttgggcttgtcatttcagtttgctgaacttttccttctggcccagattttctattttggttcataaattctattgcacaaatgtcctttattgtaaaacaccttaaattctttctaagggaaggctgcatggaaatgatacagtaaggtcttccctgcattttcttagattcctattagggaaggcagacctagatgtccctcttaccctcagtcccaaagccccccatttataaaatcccttaagcagtgactactgctgttctgagtacctggaggtagttcagagcttctgaaaggtaaaatccatatacaaaaagaagttccttccgatccagatcccagctttggtgccagatgcacatttgaggagtaggtggctagtcagactctcacctgagcagttaataaaatctattgccccttaatggaattttttctgcaagctcgaattgatctgtcatctttgtgatttgtgagatggcagggaagcaccaaacaccatcatgacttgggccacagtggtgggaaaaaaggaaaaaagaaaaaaaaaatccactgccaagccttgccaggcgtanaaagggctggaactgctggggccattttatctgatttattggaaatagagtggatcttattaacattttaataaagagaatctttttgcactaggctggaagtggccgccagtcccccgtgcaattccattctctggaaaagtggaatcagctggcattgcccagcgtgatttgtgaggctgagccccaacagtccaaagaagcaaatgggatgccacctccgcggggctcgctcctcgcgaggtgctcaccccgtatctgccatgcaaaacgagggagcgttaggaaggaatccgtcttgtaaagccattggtcctggtcatcagcctctacccaatgctttcgtgatgctgctgctgatctatttgggaagttggctggctggcgaggcagagcctctcctcaaagcctggctcccacggaaaatatgctcagtgcagccgcgtgcatgaatgaaaacgccgccgggcgcttctagtcggacaaaatgcagccgagaactccgctcgttctgtgcgttctcctgtcccaggtagggaagaggggctgccgggcgcgctctgcgccccgtttctgcattcggatcgcccggcacgggcagggtgagggggctttcggggggtcggggcctccggtcgcggcggcgaagacagatcggggctcggtagggaggtcattccgagcccagagatcctaggcaccccccacacacaggctcccactctggcgtgcgtgcgtgtgtgtgtgtgtgtgtgtgtgtgtgtgtgtgtgtgtgtgtgtgtgtacgttcgttaacgggagga

SEQ ID NO: 34 - DNA, 285 bp, homo sapiens
  exon (85)..(196)
tctaaaagttgcagatttggcgaaagttagcagggcaaacacataagaccgctattaaaatattgatggctttggttttctcaggtgctgctgctaacatctgcagaagatttggactgcactcctggatttcagcagaaagtgttccatatcaatcagccagctgaattcattgaggaccagtcaattctaaactgtaagcaatgtcactcaaagatgcttttagactcttctcatatttgaatttactaatgtttatgactgtgtctcaggatgtggtatttc

SEQ ID NO: 35 - DNA, 318 bp, homo sapiens
  exon (40)..(248): Exon 3
cctactcatgctccttctgttgtttcgtttgtttcccagtgaccttcagtgactgtaagggaaacgacaagctacgctatgaggtctcgagcccatacttcaaggtgaacagcgatggcggcttagttgctctgagaaacataactgcagtgggcaaaactctgttcgtccatgcacggaccccccatgcggaagatatggcagaactcgtgattgtcggggggaaagacatccagggctccttgcaggtaacacatctgtttgagataacttgggttcaaggaggacattaggttctgtctgtcttatgtggaaaat

SEQ ID NO: 36 - DNA, 318 bp, homo sapiens
  misc_feature (13)..(13): n is a, c, g, or t
  exon (96)..(212)
tgtatgctttccnaacaaaggaactctatctcggagcagactgatacatcatttcaatgggagattttaatcaatccttttgttttcccttttaggatatatttaaatttgcaagaacttctcctgtcccaagacaaaagaggtccattgtggtatctcccattttaattccagagaatcagagacagcctttcccaagagatgttggcaaggtaagtcagacaaacagcaaatgacaaaaacatgtttttatgaaaagatgagcacagcagactgagtatgactgtcttggtgaccagctggaattagtcttcatct

SEQ ID NO: 37 - DNA, 283 bp, homo sapiens
  misc_feature (41)..(41): n is a, c, g, or t
  exon (71)..(223): Exon 5
agtatgtttgcaatgtgctttctctgtgttttccaggcagntttaaatgtctctctgtttttctcactaggtagtcgatagtgacaggccagaaaggtccaagttccggctcactggaaagggagtggatcaagagcctaaaggaattttcagaatcaatgagaacacagggagcgtctccgtgacacggaccttggacagagaagtaatcgctgtttatcaagtgagtacccctctcccatgcccaccctgtgcgcagaaatgtggctttcaaagattgttt

SEQ ID NO: 38 - DNA, 398 bp, homo sapiens
  misc_feature (22)..(22): n is a, c, g, or t
  misc_feature (25)..(25): n is a, c, g, or t
  exon (87)..(231)
taaccatctttctctagagaanccntatttgaattttcataatgaattaatatcttctttctcccccaatctctttgctcaaatagctatttgtggagaccactgatgtcaatggcaaaactctcgaggggccggtgcctctggaagtcattgtgattgatcagaatgacaaccgaccgatctttcgggaaggcccctacatcggccacgtcatggaagggtcacccacaggtatgtcacattggcttacctttagcgtaatggcttggaaagaggcacactttgatctttgtggattctagggactgtcttatggctgttccaacttgtcagctcggtatcagcgtcgacttaatacttccctgcgtagagccaggttggaagctaagtgcaagctt

SEQ ID NO: 39 - DNA, 296 bp, homo sapiens
  exon (33)..(211): Exon 7
  misc_feature (256)..(259): n is a, c, g, or t
  misc_feature (282)..(283): n is a, c, g, or t
ccattccgtgcctttctgtcttgccccggtaggcaccacagtgatgcggatgacagcctttgatgcagatgacccagccaccgataatgccctcctgcggtataatatccgtcaacagacgcctgacaagccatctcccaacatgttctacatcgatcctgagaaaggagacattgtcactgttgtgtcacctgcgctgctggaccgagaggtgagctgaaaagaataccactttctttttcacgagaatagaatnnnncttttcatgcaatttatgatgtnnggctccagtcagt

SEQ ID NO: 40 - DNA, 367 bp, homo sapiens
  misc_feature (31)..(32): n is a, c, g, or t
  exon (35)..(175): Exon8
  misc_feature (254)..(254): n is a, c, g, or t
  misc_feature (300)..(300): n is a, c, g, or t
  misc_feature (302)..(302): n is a, c, g, or t
  misc_feature (307)..(307): n is a, c, g, or t
  misc_feature (359)..(359): n is a, c, g, or t
gctcatattatttctttgtgcttgtgctttnnagactctggaaaatcccaagtatgaactgatcatcgaggctcaagatatggctggactggatgttggattaacaggcacggccacagccacgatcatgatcgatgacaaaaatgatcactcaccaaaattcaccaagaaagaggtaaacccctgtgccaaacaccaaccaccactgtggtcacagctacaattactgattgatgttaattcacgtaccacgncacttgctggcccccctttcaaaatcaaaatactcctttgtgggananacganggtgttttgctggaattctttctcacatttttattttgtgtttgtgtgtgangctaaaat

SEQ ID NO: 41 - DNA, 603 bp, homo sapiens
  exon (213)..(395): Exon 9
cccacattctgcaggagagaagttagtctcgtggcctcacctaagttcaaggaaagctgagaagaagagcatagtatcttccaaccgtaatcctcttatgttattatttatttttaagtgagatgatgtgtgtaacatacccaatgctttgcatgtagtaaatgactatgtgttttcaaaatagtgaccattaccatctgctttgtttgcagtttcaagccacagtcgaggaaggagctgtgggagttattgtcaatttgacagttgaagataaggatgaccccaccacaggtgcatggagggctgcctacaccatcatcaacggaaaccccgggcagagctttgaaatccacaccaaccctcaaaccaacgaagggatgctttctgttgtcaaagtaagggtgcttccaattgcctctttctcctcatgcgagcacggagggccccatgaggcagctcatagaatcattgagtttagaaggccacagataaattcccccagtctcctccttctgggaattaacaaaggaaaaggctcatggcttaagggtgcactcgtatcctcagcagcataaacaagcagccataaaacaaacctgtttt

SEQ ID NO: 42 - DNA, 415 bp, homo sapiens
  misc_feature (5)..(5): n is a, c, g, or t
  misc_feature (18)..(18): n is a, c, g, or t
  exon (56)..(309): Exon 10
ccacntgccttttgtggntggtgtgcatcctgagacccttctgtctgctttccagccattggactatgaaatttctgccttccacaccctgctgatcaaagtggaaaatgaagacccactcgtacccgacgtctcctacggccccagctccacagccaccgtccacatcactgtcctggatgtcaacgagggcccagtcttctacccagaccccatgatggtgaccaggcaggaggacctctctgtgggcagcgtgctgctgacagtgaatgccacggaccccgactccctgcagcatcaaaccatcaggtgggtgagtggctccggaaccacagacgggaggtgggcaggaatggcttttcctttccagtcgcagaagctggttgtcaggagcagacaccattaaattaaactt

SEQ ID NO: 43 - DNA, 280 bp, homo sapiens
  exon (54)..(196): Exon 11
tttcttgaatctactttgaatgcagagtctgacattgtggggatttttttcaggtattctgtttacaaggacccagcaggttggctgaatattaaccccatcaatgggactgttgacaccacagctgtgctggaccgtgagtccccatttgtcgacaacagcgtgtacactgctctcttcctggcaattgacagtggtgagtacttgacaaagaccatcaagggtatacttttctgctacaaatatacttttacatttttaaatttcttctgatacaccc

SEQ ID NO: 44 - DNA, 451 bp, homo sapiens
  misc_feature (30)..(30): n is a, c, g, or t
  exon (171)..(404): Exon 12
  misc_feature (428)..(428): n is a, c, g, or t
tgtagcatataccagttgcataccaacatnttccctttttcccacaggcaaccctcccgctacgggcactgggactttgctgataaccctggaggacgtgaatgacaatgccccgttcatttaccccacagtagctgaagtctgtgatgatgccaaaaacctcagtgtaggcaaccctcccgctacgggcactgggactttgctgataaccctggaggacgtgaatgacaatgccccgttcatttaccccacagtagctgaagtctgtgatgatgccaaaaacctcagtgtagtcattttgggagcatcagataaggatcttcacccgaatacagatcctttcaaatttgaaatccacaaacaagctgttcctgataaagtctggaagatctccaagatcaacagtaagtctggctaaagcatttctncctattcttccagctttcagttt

SEQ ID NO: 45 - DNA, 442 bp, homo sapiens
  exon (160)..(378): Exon 13
  misc_feature (398)..(398): n is a, c, g, or t
  misc_feature (422)..(422): n is a, c, g, or t
  misc_feature (429)..(429): n is a, c, g, or t
tggtattttcacttgatgttaatgaatttaaatgtaagcattcgcacaattataaatgaaaatgcaatgcctgtttggtgtgactttcatcaccaaaacctcatcttttattggaaaaagtctcatccactctcaccagaaccctccttgcctttacagatacacacgccctggtaagccttcttcaaaatctgaacaaagcaaactacaacctgcccatcatggtgacagattcagggaaaccacccatgacgaatatcacagatctcagggtacaagtgtgctcctgcaggaattccaaagtggactgcaacgcggcgggggccctgcgcttcagcctgccctcagtcctgctcctcagcctcttcagcttagcttgtaagttgacctaactccantgcatgcacaaacaggaaattgtnacttcantgggttcgtgaag

SEQ ID NO: 46 - DNA, 1329 bp, homo sapiens
  misc_feature (45)..(45): n is a, c, g, or t
  misc_feature (184)..(186): n is a, c, g, or t
  misc_feature (190)..(190): n is a, c, g, or t
  misc_feature (201)..(204): n is a, c, g, or t
  exon (210)..(217): Exon 14
gcattttcagagaagtccagctttttaccatataaccattcattntgtcttccaatgtgatccttaaggaaaaaaaaaattcccccatagtcgtgtgatgtttaaaatgtgtttattataccttgcctgtcatttttctggcttttagctaaaatgttttgaattgagtggtgatattcccgannnaacnctgaaccctcnnnnttcaggtctgtgagaactcctgacgtctgaagcttgactcccaagtttccatagcaacaggaaaaaaaaaaatctatccaaatctgaagattgcggtttacagctatcgaacttcacaactaggcctcaattgttccggttttttattttctttacaatttcacttagtctgtacttcatcattttgacagcatcttcctccctcctttaattaatggaatcttctgaattttccctgaatgtttaaagatcatgacatatgacttgatcttctgggagcaggaacaatgactactttttctggtgtgttaacatgtcgctagccagtgctccaggcacccagctttgtctgtgggttagtattggtgtatgtatgagtatctgtatgtatatatacacggtatttatagagagagactatcctggagaagcctcgttttgatgccattcttccttgcaaggttaagcaaggtgggtggaaactaagacacctgaaccctccagggcctcccgcatcaaggtcagcatgaggacagaccacagagctgtcacttttgctccgaagctacttctccactgtcccgttcagtctgaatctgccacaaccagccaggcaggtccacagagagggagagcagagaaagaagtcctttctctttattgagttcgaggactacaaccaatttacactgccatctgatgccgtgatcctgagccaaggaggtgaggagcagagcaggcaatttcaccaccaaatgccaagaaaagggctgacattttctttcatgggcaccaacctgcatttgtatgtgtcccgaatccacagtcgtactgattctaatggggacacagatcatggtagagaatctctccctcctcagtaaatgtacaactgcacctgtcatcatggaggtcatacatgcatacaaagaggtgtacaggtaccatcttgtatacacatatatacccacatgtacagacatacatttatgcacattcacgctgtttgtttcatatatacaggcataaaatagagtaaatacaggtagttttaaaagtacccttttgtgtgaattgactaccgttgtttgcaaacccgaaaataaaagacgttcattatgtatg

SEQ ID NO: 47 - DNA, 2333 bp, mus musculus
  CDS (114)..(2255)
ccacgcgtccgggcagagcctctcctcaaaggctggctcccacggaaaatatgctcagtgcagccgcgtgtgtgaatgcaaacgccgccaggcgcttcttctagtcgggcaagatgcagccgagaactccgctcaccctgtgcgtcctgctgtcccaggtgctcctggtcacgtctgcagatgatctggagtgcacccctggattccagcagaaagtgttacacatccaccagcctgccgaattcatcgaggaccagcctgtcctaaacttgaccttcagtgactgcaagggcaacgagaagctgcactacgaggtctcaagtccacacttcaaggtgaacagcgatggcaccttagtggctctcaggaacatcactgcggtgggcaggaccctgtttgtccatgcgaggactcctcatgctgaagacatggcagaactcgtgattgtcgggggcaaagacatccagggctccttgcaggatatctttaaatttgcaaggacatctcctgtcccaagacaaaagaggtccattgtggtgtcccccatcttgatcccagagaaccagagacagcccttccccagagatgtcggcaaggtagtcgatagcgacagacccgaagggtccaagttccggctcaccgggaagggagtggatcaagaccctaaaggaactttcagaatcaatgagaacacaggcagcgtctccgtgacacggaccctggacagagaaacgatcgctacttatcaactgtatgtggaaaccacggatgccagtggcaaaactctggaagggccagtgcctctggaagtcattgtgattgaccagaacgacaacagacccatcttccgggaaggcccttacatcggccatgtcatggaagggtcacccacagggaccacggtgatgcggatgacagcgtttgatgcagatgacccggctactgacaatgctctcctgaggtacaacatccgtcagcagacgcctgacaagccatctcctaacatgttctacatcgatcccgagaaaggagacattgtcaccgtggtgtcacctgcgctgctggaccgggagactctggaaaaccccaagtatgaactgatcatcgaagctcaagatatggcaggattggatgtcggactgacaggcacagccacagccaccatcgtgatcgatgacaaaaatgatcactcaccaaaattcaccaagaaagagtttcaagccacagtggaggagggagctgtgggggtcattgtcaacttgacagtggaagacaaagacgaccccaccacaggagcctggagggctgcatacaccatcatcaatggaaatcctgggcagagcttcgagatccacaccaacccacagaccaacgagggcatgctctctgtggtcaagcccctggactatgagatctctgcctttcacaccctgctgatcaaagtggagaatgaggacccactggtacccgacgtctcctatggccccagctccacggcaactgtccacatcacagtcctggatgtcaacgagggaccagtcttctacccggaccccatgatggtgactaaacaggagaacatctctgtgggcagtgtgctgctgacagtgaatgccacagacccagactccctgcaacatcaaactatcaggtactctgtctacaaggacccagcaggctggctgagtatcaaccccatcaatggaacggtggacaccacagctgtgttggaccgggagtccccattcgtccacaacagtgtgtacacggccctgttcctggccatcgacagtggcaaccctcctgcaactggcaccgggactctgctgataaccctagaggacattaatgacaacgctcctgtcatttacccaactgtggctgaggtctgcgatgatgccagaaacctcagcgtggtcattctgggggcttcagacaaggaccttcaccccaacacagaccccttcaagtttgagattcataaacagacagtccctgataaagtttggaagatctccaaaatcaacaacactcacgccctcgtgagccttcttcagaatctgaacaaggcaaactacaacctgcccatcatggtgacagattcagggaagccacccatgacgaacatcacggacctcagggtgcaggtgtgctcttgcaagaactccaaagtggactgcaacggggctggggccctgcacctcagcctcagcttgctgctgctcttctctctcctcagcttattgtcaggtctgtgagaacgacacgcctgaagcttgactcccaagttgccatagcaacaggaaaaaaaaaaaaaaaaaaaaaaaaaaaaa

SEQ ID NO: 48 - protein, 714 aa, mus musculus
MQPRTPLTLCVLLSQVLLVTSADDLECTPGFQQKVLHIHQPAEFIEDQPVLNLTFSDCKGNEKLHYEVSSPHFKVNSDGTLVALRNITAVGRTLFVHARTPHAEDMAELVIVGGKDIQGSLQDIFKFARTSPVPRQKRSIVVSPILIPENQRQPFPRDVGKVVDSDRPEGSKFRLTGKGVDQDPKGTFRINENTGSVSVTRTLDRETIATYQLYVETTDASGKTLEGPVPLEVIVIDQNDNRPIFREGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIVIDDKNDHSPKFTKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTKQENISVGSVLLTVNATDPDSLQHQTIRYSVYKDPAGWLSINPINGTVDTTAVLDRESPFVHNSVYTALFLAIDSGNPPATGTGTLLITLEDINDNAPVIYPTVAEVCDDARNLSVVILGASDKDLHPNTDPFKFEIHKQTVPDKVWKISKINNTHALVSLLQNLNKANYNLPIMVTDSGKPPMTNITDLRVQVCSCKNSKVDCNGAGALHLSLSLLLLFSLLSLLSGL

SEQ ID NO: 49 - DNA, 2305 bp, mus musculus
  CDS (109)..(2253)
ctggcgaggcatagcctctcctcaaaggctggctcccacggaaaatatgctcagtgcagccgcgtgtgtgaatgcaaacgccgccaggctttcttctagtcgggcaagatgcagccgagaactccgctcaccctgtgcgtcctgctgtcccaggtgctcctggtcacgtctgcagatgatctggagtgcacccctggattccagcggaaagtgttacacatccaccagcctgccgaattcatcgaggaccagcctgtcctaaacttgaccttcaatgactgcaagggcaacgagaagctgcactacgaggtctcaagcccacacttcaaggtgaacagcgacggcaccttagtggctctcaggaacatcactgcggtgggcaggaccctgtttgtccatgcgaggactcctcatgctgaagacatggcagaactcgtgattgtcgggggcaaagacatccagggctccttgcaggatatctttaaatttgcaaggacatctcctgtcccaagacaaaagaggtccattgtggtgtcccccatcttgatcccagagaaccagagacagcccttccccagagatgtcggcaaggtagtcgatagcgacagacccgaagggtccaagttccggctcaccgggaagggagtggatcaagaccctaaaggaactttcagaatcaatgagaacacaggcagtgtctccgtgacacggaccctggacagagaaacgatcgctacttatcaactgtatgtggaaaccacggatgccagtggcaaaactctggaagggccagtgcctctggaagtcattgtgattgaccagaacgacaacagacccatcttccgggaaggcccttacatcggccatgtcatggaagggtcacccacagggaccacggtgatgcggatgacagcgtttgatgcagatgacccggctactgacaatgctctctggaggtacaacatccgtcagcagacgcctgacaagccatctcctaacatgttctacatcgatcccgagaaaggagacattgtcaccgtggtgtcacctgcgctgctggaccgggagactctggaaaaccccaagtatgaactgatcatcgaagctcaagatatggcaggattggatgtcggactgacaggcacagccacagccaccatcgtgatcgatgacaaaaatgatcactcaccaaaattcaccaagaaagagtttcaagccagagtggaggagggagctgtgggggtcattgtcaacttgacagtggaagacaaagacgaccctaccacaggagcctggagggctgcatacaccatcatcaatggaaatcctgggcagagcttcgagatccacaccaacccacagaccaacgagggcatgctctctgtggtcaagcccctggactatgagatctctgcctttcacaccctgctgatcaaagtggagaatgaggacccactggtacccgacgtctcctatggccccagctccacggcaactgtccacatcacagtcctggatgtcaacgagggaccagtcttctacccggaccccatgatggtgactaaacaggagaacatctctgtgggcagtgtgctgctgacagtgaatgccacagacccagactccctgcaacatcaaactatcaggtactctatctacaaggacccagcaggctggctgagtatcaaccccatcaatggaacggtggacaccacagctgtgttggaccgggagtccccattcgtccacaacagtgtgtacacggccctgttcctggccatcgacagtggcaaccctcctgcaactggcaccgggactctgctgataaccctagaggacattaatgacaacgctcctgtcatttacccaactgtggctgaggtctgcgatgatgccagaaacctcagcgtggtcattctgggggcttcggacaaggaccttcaccccaacacagaccccttcaagtttgagattcataaacagacagtccctgataaagtctggaagatctccaaaatcaacaacacccacgccctcgtgagccttcttcagaatctgaacaaggcaaactacaacctgcccatcatggtgacagattcagggaagccacccatgacgaacatcacggacctcaaggtgcaggtgtgctcttgcaagaactccaaagtggactgcaacggggctggggccctgcacctcagcctcagcttgctgctgctcttctctctcctcagcttattgtcaggtctgtgagaacgacacgcctgaagcttgactcccaagttgccatagcaacaggaaaaag

SEQ ID NO: 50 - protein, 714 aa, mus musculus
MQPRTPLTLCVLLSQVLLVTSADDLECTPGFQRKVLHIHQPAEFIEDQPVLNLTFNDCKGNEKLHYEVSSPHFKVNSDGTLVALRNITAVGRTLFVHARTPHAEDMAELVIVGGKDIQGSLQDIFKFARTSPVPRQKRSIVVSPILIPENQRQPFPRDVGKVVDSDRPEGSKFRLTGKGVDQDPKGTFRINENTGSVSVTRTLDRETIATYQLYVETTDASGKTLEGPVPLEVIVIDQNDNRPIFREGPYIGHVMEGSPTGTTVMRMTAFDADDPATDNALWRYNIRQQTPDKPSPNMFYIDPEKGDIVTVVSPALLDRETLENPKYELIIEAQDMAGLDVGLTGTATATIVIDDKNDHSPKFTKKEFQARVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTKQENISVGSVLLTVNATDPDSLQHQTIRYSIYKDPAGWLSINPINGTVDTTAVLDRESPFVHNSVYTALFLAIDSGNPPATGTGTLLITLEDINDNAPVIYPTVAEVCDDARNLSVVILGASDKDLHPNTDPFKFEIHKQTVPDKVWKISKINNTHALVSLLQNLNKANYNLPIMVTDSGKPPMTNITDLKVQVCSCKNSKVDCNGAGALHLSLSLLLLFSLLSLLSGL

SEQ ID NO: 51 - DNA, 2420 bp, mus musculus
  CDS (224)..(2365)
aaaacgagggagcgttatgaaggaatccgtcttgtaaagccattggtcctggtcatcagccgctacccaatgctttcgcgatgctgccgctgatctatttgggaagttggctggctggcgaggcagagcctctcctcaaaggctggctcccacggaaaatatgctcagtgcagccgcgtgtgtgaatgcaaacgccgccaggcgcttcttctagtcgggcaagatgcagccgagaactccgctcaccctgtgcgtcctgctgtcccaggtgctcctggtcacgtctgcagatgatctggagtgcacccctggattccagcggaaagtgttacacatccaccagcctgccgaattcatcgaggaccagcctgtcctaaacttgaccttcaatgactgcaagggcaacgagaagctgcactacgaggtctcaagcccacacttcaaggtgaacagcgacggcaccttagtggctctcaggaacatcactgcggtgggcaggaccctgtttgtccatgcgaggactcctcatgctgaagacatggcagaactcgtgattgtcgggggcaaagacatccagggctccttgcaggatatctttaaatttgcaaggacatctcctgtcccaagacaaaagaggtccattgtggtgtcccccatcttgatcccagagaaccagagacagcccttccccagagatgtcggcaaggtagtcgatagcgacagacccgaagggtccaagttccggctcaccgggaagggagtggatcaagaccctaaaggaactttcagaatcaatgagaacacaggcagtgtctccgtgacacggaccctggacagagaaacgatcgctacttatcaactgtatgtggaaaccacggatgccagtggcaaaactctggaagggccagtgcctctggaagtcattgtgattgaccagaacgacaacagacccatcttccgggaaggcccttacatcggccatgtcatggaagggtcacccacagggaccacggtgatgcggatgacagcgtttgatgcagatgacccggctactgacaatgctctcctgaggtacaacatccgtcagcagacgcctgacaagccatctcctaacatgttctacatcgatcccgagaaaggagacattgtcaccgtggtgtcacctgcgctgctggaccgggagactctggaaaaccccaagtatgaactgatcatcgaagctcaagatatggcaggattggatgtcggactgacaggcacagccacagccaccatcgtgatcgatgacaaaaatgatcactcaccaaaattcaccaagaaagagtttcaagccacagtggaggagggagctgtgggggtcattgtcaacttgacagtggaagacaaagacgaccctaccacaggagcctggagggctgcatacaccatcatcaatggaaatcctgggcagagcttcgagatccacaccaacccacagaccaacgagggcatgctctctgtggtcaagcccctggactatgagatctctgcctttcacaccctgctgatcaaagtggagaatgaggacccactggtacccgacgtctcctatggccccagctccacggcaactgtccacatcacagtcctggatgtcaacgagggaccagtcttctacccggaccccatgatggtgactaaacaggagaacatctctgtgggcagtgtgctgctgacagtgaatgccacagacccagactccctgcaacatcaaactatcaggtactctatctacaaggacccagcaggctggctgagtatcaaccccatcaatggaacggtggacaccacagctgtgttggaccgggagtccccattcgtccacaacagtgtgtacacggccctgttcctggccatcgacagtggcaaccctcctgcaactggcaccgggactctgctgataaccctagaggacattaatgacaacgctcctgtcatttacccaactgtggctgaggtctgcgatgatgccagaaacctcagcgtggtcattctgggggcttcggacaaggaccttcaccccaacacagaccccttcaagtttgagattcataaacagacagtccctgataaagtctggaagatctccaaaatcaacaacacccacgccctcgtgagccttcttcagaatctgaacaaggcaaactacaacctgcccatcatggtgacagattcagggaagccacccatgacgaacatcacggacctcagggtgcaggtgtgctcttgcaagaactccaaagtggactgcaacggggctggggccctgcacctcagcctcagcttgctgctgctcttctctctcctcagcttattgtcaggtctgtgagaacgacacgcctgaagcttgactcccaagttgccatagcaacaggaaaaag

SEQ ID NO: 52 - protein, 714 aa, mus musculus
MQPRTPLTLCVLLSQVLLVTSADDLECTPGFQRKVLHIHQPAEFIEDQPVLNLTFNDCKGNEKLHYEVSSPHFKVNSDGTLVALRNITAVGRTLFVHARTPHAEDMAELVIVGGKDIQGSLQDIFKFARTSPVPRQKRSIVVSPILIPENQRQPFPRDVGKVVDSDRPEGSKFRLTGKGVDQDPKGTFRINENTGSVSVTRTLDRETIATYQLYVETTDASGKTLEGPVPLEVIVIDQNDNRPIFREGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIVIDDKNDHSPKFTKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVFYPDPMMVTKQENISVGSVLLTVNATDPDSLQHQTIRYSIYKDPAGWLSINPINGTVDTTAVLDRESPFVHNSVYTALFLAIDSGNPPATGTGTLLITLEDINDNAPVIYPTVAEVCDDARNLSVVILGASDKDLHPNTDPFKFEIHKQTVPDKVWKISKINNTHALVSLLQNLNKANYNLPIMVTDSGKPPMTNITDLRVQVCSCKNSKVDCNGAGALHLSLSLLLLFSLLSLLSGL

SEQ ID NO: 53 - DNA, 2305 bp, mus musculus; identical to SEQ ID NO: 49
  CDS (109)..(2250)

SEQ ID NO: 54 - protein, 714 aa, mus musculus; identical to SEQ ID NO: 50

SEQ ID NO: 55 - protein, 103 aa, homo sapiens
SPILIPENQRQPFPRDVGKVVDSDRPERSKFRLTGKGVDQEPKGIFRINENTGSVSVTRTLDREVIAVYQLFVETTDVNGKTLEGPVPLEVIVIDQNDNRPIF

SEQ ID NO: 56 - protein, 118 aa, homo sapiens
REGPYIGHVMEGSPTGTTVMRMTAFDADDPATDNALLRYNIRQQTPDKPSPNMFYIDPEKGDIVTVVSPALLDRETLENPKYELIIEAQDMAGLDVGLTGTATATIMIDDKNDHSPKF

SEQ ID NO: 57 - protein, 114 aa, homo sapiens
TKKEFQATVEEGAVGVIVNLTVEDKDDPTTGAWRAAYTIINGNPGQSFEIHTNPQTNEGMLSVVKPLDYEISAFHTLLIKVENEDPLVPDVSYGPSSTATVHITVLDVNEGPVF

SEQ ID NO: 58 - protein, 108 aa, homo sapiens
YPDPMMVTRQEDLSVGSVLLTVNATDPDSLQHQTIRYSVYKDPAGWLNINPINGTVDTTAVLDRESPFVDNSVYTALFLAIDSGNPPATGTGTLLITLEDVNDNAPFI

SEQ ID NO: 59 - protein, 95 aa, homo sapiens
YPTVAEVCDDAKNLSVVILGASDKDLHPNTDPFKFEIHKQAVPDKVWKISKINNTHALVSLLQNLNKANYNLPIMVTDSGKPPMTNITDLRVQVC